(19) United States
(12) Patent Application Publication
(10) Pub. No.: US 2015/0118215 A1
(43) Pub. Date: Apr. 30, 2015

(54) Methods of Inhibiting Cell Proliferation

(71) Applicant: The General Hospital Corporation, Boston, MA (US)

(72) Inventor: Vladimir Marshansky, Sharon, MA (US)

(21) Appl. No.: 14/388,906

(22) PCT Filed: Mar. 14, 2013
(86) PCT No.: PCT/US13/31739

§ 371 Date: Sep. 29, 2014

Related U.S. Application Data
(60) Provisional application No. 61/618,231, filed on Mar. 30, 2012.

(51) Int. Cl. A61K 38/46; A61K 38/28

(52) U.S. Cl. CPC A61K 38/46; A61K 38/28
USPC 424/94.6

(57) ABSTRACT

Described are methods of inhibiting cytohesin-2 to treat cancer, treat diabetic nephropathy, and deliver a compound into a cell.

### CLAIM OF PRIORITY

This application claims the benefit of U.S. Patent Application No. 61/618,231, filed on Mar. 30, 2012, the entire contents of which are hereby incorporated by reference.

### FEDERALLY SPONSORED RESEARCH OR DEVELOPMENT

This invention was made with U.S. Government support under Grant No. DK038452 awarded by the National Institutes of Health. The Government has certain rights in the invention.

### TECHNICAL FIELD

This invention relates to methods of inhibiting cytohesin-2 to treat cancer, treat diabetic nephropathy, and deliver a compound into a cell.

### BACKGROUND

Cytohesin-family guanine-nucleotide exchange factors (GEFs) include four members: i) cytohesin-1, ii) cytohesin-2 (CTH2) (also known as ARNO, ADP-ribosylation factor nucleotide site opener); iii) cytohesin-3 (also called ARNO3 or GRP1) and cytohesin-4. Their structure is divided into the following four regions: i) an N-terminal coiled-coil (CC); ii) a catalytic Sec7 domain; iii) a pleckstrin homology (PH) domain; and iv) a C-terminal polybasic (PB) motif region. The Sec7 domain of cytohesin-2 is a major binding site of interaction and signaling with V-ATPase (Merkulova et al. (Biochim Biophys Acta 1797, 1398-1409, 2010)). The conserved Sec7 catalytic domain is responsible for GDP/GTP-exchange and activation of Arf GTP-binding proteins (Arfs). These proteins belong to the Ras-superfamily small GTPases, which regulate an extraordinary variety of cell functions (Bourne et al. (Nature 348, 125-132, 1990)). They function as “molecular switches” in which transition between “on” and “off” states of this molecular device is mediated by a GDP/GTP cycle. Six members of the Arf-family (Arf1-Arf6) have been identified in mammalian cells (Donaldson and Jackson (Curr Opin Cell Biol 12, 475-482, 2000)). Initial studies implicated cytohesins and Arfs in regulation of the vesicular trafficking in both endocytic and exocytic pathways. In particular, their regulation of endosomal/lysosomal protein degradation pathway is exerted via: i) interaction with V-ATPase; ii) recruitment of coat components; iii) modification of phospholipids; iv) remodeling of the actin cytoskeleton; and v) control of microtubule-dependent vesicular trafficking. In addition to their role as Arf-GEFs, cytohesins have also emerged as transcriptional regulators (Chen et al. (J Biol Chem 281, 19985-19994, 2006)). In particular, cytohesin-2 is involved in regulation of gene expression via the MAPK signaling pathway during serum-mediated transcriptional activation (Theis et al. (Proc Natl Acad Sci USA 101, 11221-11226, 2004)). Cytohesin-2 has recently emerged as a crucial regulator of signaling transmembrane receptors (Bill et al. (Cell 143, 201-211, 2010); Hafner et al. (Nature 444, 941-944, 2006); Fuss et al. (Nature 444, 945-948, 2006)).

### SUMMARY

The present invention is based, at least in part, on the discovery of the regions of V-ATPase and cytohesin-2 that interact, and a1-, a2-, a3-, and a4-subunit derived peptides that are able to inhibit the function of the enzymatic GEF-activity of cytohesin-2 within the V-ATPase/small GTPase complex. These peptides can be used as a therapeutic agent in the treatment of a number of conditions, including cancer and diabetic nephropathy.

Accordingly, in one aspect the present disclosure provides methods of treating cancer, e.g., an epithelial cancer or carcinoma, e.g., lung cancer, non-small cell lung cancer, pancreatic cancer, squamous cell carcinomas of the head and neck, prostate cancer, breast cancer, colon cancer, kidney cancer, liver cancer, and brain cancer. The methods may include selecting a subject having or at risk for developing cancer; and administering to the subject a therapeutically effective amount of a cytohesin-2 inhibitor, wherein the cytohesin-2 inhibitor comprises: a peptide consisting of an amino acid sequence that has at least 90% identity to the amino acid sequence of SEQ ID NO:9, 11, 13, 15, 25, 27, 29, or 31; a fusion polypeptide comprising a first portion consisting of an amino acid sequence that has at least 90% identity to the amino acid sequence of SEQ ID NO:9, 11, 13, 15, 25, 27, 29, or 31; and at least a second portion linked to the first portion, wherein the second portion comprises one or more non-VATPase sequences; or any combination thereof, to thereby treat cancer in the subject. In some embodiments, the second portion of the fusion polypeptide can include a cell-penetrating peptide, e.g., a TAT peptide, or a small molecule, e.g., SecinH3, Secin16, Secin69, Secin107, and Secin132. In some embodiments, the methods include treating the subject, e.g., a human, with chemotherapy.

In another aspect, the disclosure features methods of treating or reducing a risk of developing diabetic nephropathy, the method comprising selecting a subject having or at risk for developing diabetic nephropathy; and administering to the subject a therapeutically effective amount of a cytohesin-2 inhibitor, wherein the cytohesin-2 inhibitor comprises: a peptide consisting of an amino acid sequence that has at least 90% identity to the amino acid sequence of SEQ ID NO:9, 11, 13, 15, 25, 27, 29, or 31; a fusion polypeptide comprising a first portion consisting of an amino acid sequence that has at least 90% identity to the amino acid sequence of SEQ ID NO:9, 11, 13, 15, 25, 27, 29, or 31; and at least a second portion linked to the first portion, wherein the second portion comprises one or more non-VATPase sequences; or any combination thereof, to thereby treat or reduce a risk of developing diabetic nephropathy in the subject. In some embodiments, the second portion of the fusion polypeptide can include a cell-penetrating peptide, e.g., a TAT peptide, or a small molecule, e.g., SecinH3, Secin16, Secin69, Secin107, and Secin132. In some embodiments, the subject, e.g., a human, has type 1 diabetes. In one embodiment, the method includes administering insulin to the subject.

In yet a further aspect, the present disclosure features methods of delivering a compound into a cell. For example, the method includes providing a cell; and contacting the cell with: a fusion polypeptide comprising a first portion consisting of an amino acid sequence that has at least 90% identity to the amino acid sequence of SEQ ID NO: 11 or 27; and at least a second portion linked to the first portion, wherein the second portion comprises a TAT peptide; and a compound selected from the group consisting of a nucleic acid, amino acid, peptide, polypeptide, antibody, small molecule, toxin, and nanoparticle. In some embodiments, the cell is contacted with a fusion polypeptide comprising a first portion consisting of an amino acid sequence that has at least 90% identity to the amino acid sequence of SEQ ID NO: 11; and at least a second portion linked to the first portion, wherein the second portion comprises a TAT peptide; and a fusion polypeptide comprising a first portion consisting of an amino acid sequence that has at least 90% identity to the amino acid sequence of SEQ ID NO:27; and at least a second portion linked to the first portion, wherein the second portion comprises a TAT peptide. In some embodiments, the compound is linked, e.g., covalently linked, to the fusion polypeptide. In any of the methods described herein, the second portion of the fusion polypeptide can include a cell-penetrating peptide, e.g., a TAT peptide, or a small molecule, e.g., SecinH3, Secin16. Secin69, Secin107, and Secin132.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control. Other features and advantages of the invention will be apparent from the following detailed description and figures, and from the claims.

### DESCRIPTION OF DRAWINGS

FIG. 1 is a schematic drawing of the EGFR/ErbB/cytohesin-2N-ATPase/aldolase pathway, and its role in signaling and tracking in the endosomal/lysosomal pathway.

FIG. 2A is a photomicrograph depicting preparation of recombinant myr-Arf1 and myr-Arf6 GTP-binding proteins.

FIG. 2B is a line graph showing a time-course of nucleotide exchange on myr-Arf1 and myr-Arf6 catalyzed by cytohesin-2. Exchange was followed by measuring the binding of [³⁵S]-GTPγS in an assay mixture that included PIP₂-containing liposomes.

FIG. 2C is a line graph showing dose-dependent inhibition of enzymatic GEF-activity of cytohesin-2 with myr-Arf6 by V-ATPase-derived a2N₁₋₁₇ peptide.

FIG. 2D is a line graph showing dose-dependent inhibition of enzymatic GEF-activity of cytohesin-2 with myr-Arf1 by V-ATPase-derived a2N₁₋₁₇ peptide.

FIG. 2E is a line graph showing dose-dependent inhibition by SecinH3 (IC₅₀>150 μM) of enzymatic GEF-activity of cytohesin-2 with myr-Arf6.

FIG. 2F is a line graph showing dose-dependent inhibition by SecinH3 (IC₅₀>150 μM) of enzymatic GEF-activity of cytohesin-2 with myr-Arf1.

FIG. 3A is a line graph showing fluorescent spectra of [Δ17]Arf1 loaded with either GTPγS or GDP.

FIG. 3B is a line graph showing dependence of GEF activity on concentration of Sec7 domain.

FIG. 3C is a line graph showing time-course of enzymatic GEF-activity of Sec7 with [Δ17]Arf1 and dose-dependent inhibition by V-ATPase-derived a2N₁₋₁₇ peptide.

FIG. 3D is a line graph showing dose-dependent inhibition of enzymatic GEF-activity of Sec7 with [Δ17]Arf1 by V-ATPase-derived a2N₁₋₁₇ peptide.

FIG. 3E is a line graph showing time-course of enzymatic GEF-activity of Sec7 with [Δ17]Arf1 and dose-dependent inhibition by SecinH3.

FIG. 3F is a line graph showing dose-dependent inhibition (IC₅₀=6.9 μM) of enzymatic GEF-activity of Sec7 with [Δ17]Arf1 by SecinH3.

FIG. 4A is an alignment of the first twenty amino acids of eukaryotic a-subunit isoforms of V-ATPase showing the conserved character of binding surface-forming amino acids F₅, E₈, M₁₀, and Q₁₄ from yeast to humans.

| Species | Sequence | SEQ ID NO: |
|---|---|---|
| Yeast STVI | MNQEEAIFRSADMTYVQLYI | 35 |
| | IPLE | |
| Yeast VPHI | MAEKEEAIFRSAEMALVQFY | 36 |
| | IPQE | |
| Fly Vha100-1 | MGSLFRSEEMALCQLFLQSE | 37 |
| Fly Vha100-2 | MGDMFRSEEMALCQMFIQPE | 38 |
| Fly Vha100-3 | MRVFKKRQTKVKSFFRSEDM | 39 |
| | DLCQLLLHTE | |
| Fly Vha100-4 | MSKWWSCGSNQESNSIFRSE | 40 |
| | VMSLVQMYLQPE | |
| Fly Vha100-5 | MGDMERSEKMALCQLFIQPE | 41 |
| Zebrafish a1 | MGELFRSEEMTLAQLFLQSE | 42 |
| Zebrafish a2 | MGSLFRSEEMCLAQLFLQSG | 43 |
| Mouse a1 | MGELFRSEEMTLAQLFLQSE | 44 |
| Mouse a2 | MGSLFRSESMCLAQLFLQSG | 45 |
| Mouse a3 | MGSMFRSEEVAIAQLLITIG | 46 |
| Mouse a4 | MASVFRSEEMCLSQVFLQVE | 47 |
| Human a1 | MGELFRSEEMTLAQLFLQSE | 48 |
| Human a2 | MGSLFRSETMCLAQLFLQSG | 49 |
| Human a3 | MGSMFRSEEVALVQLFLPTA | 50 |
| Human a4 | MVSVFRSEEMCLSQLFLQVE | 51 |

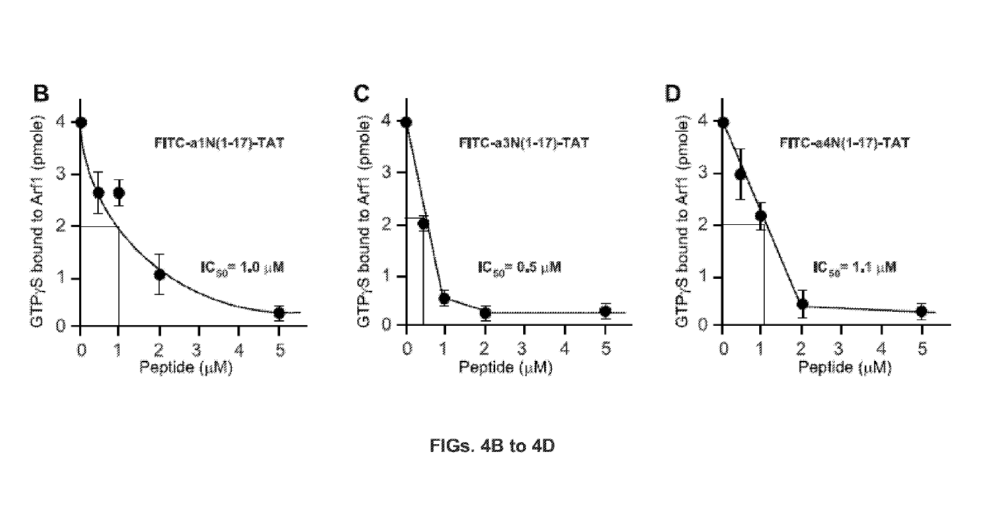

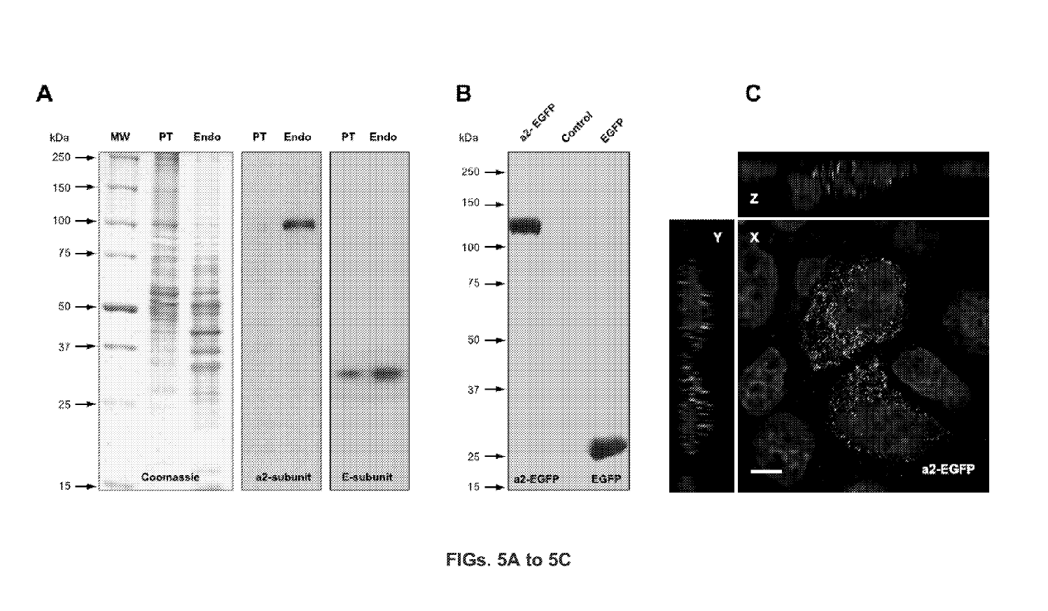

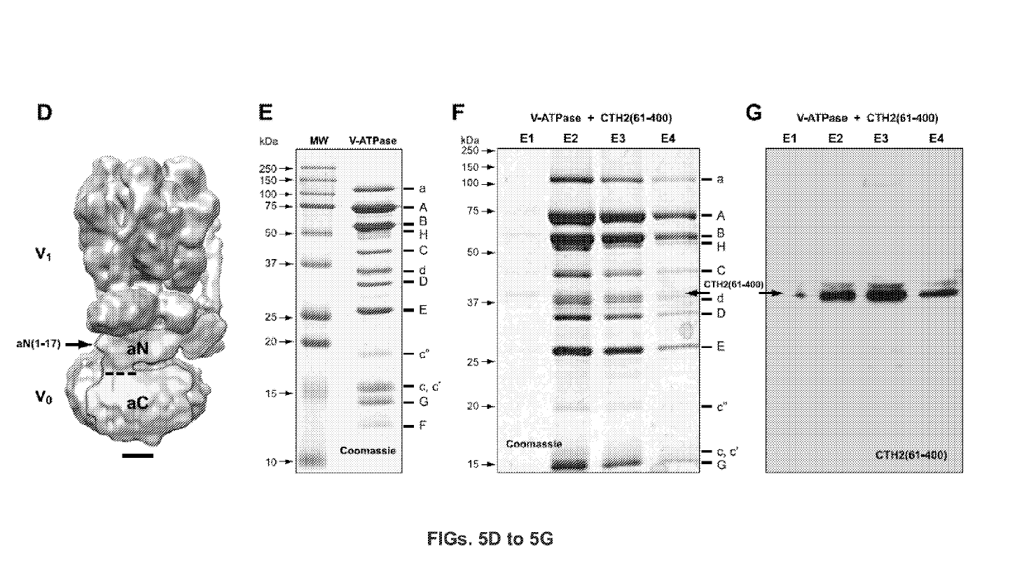

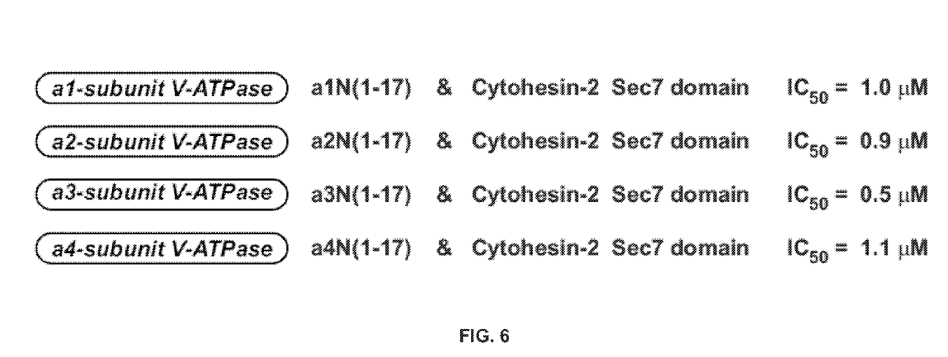

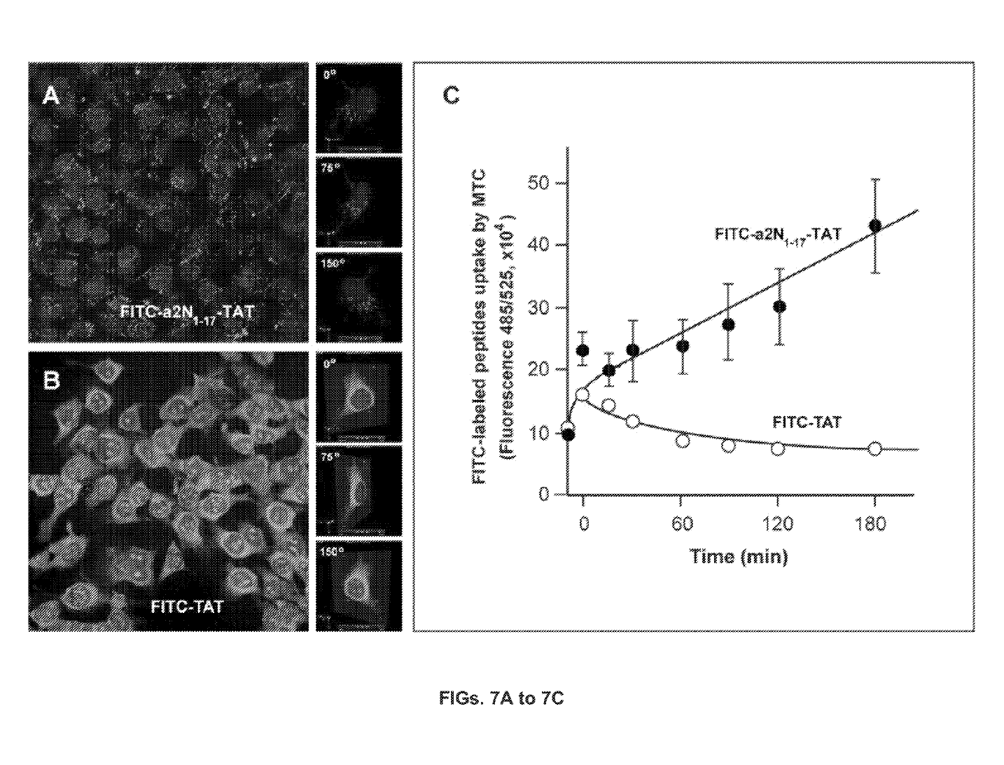

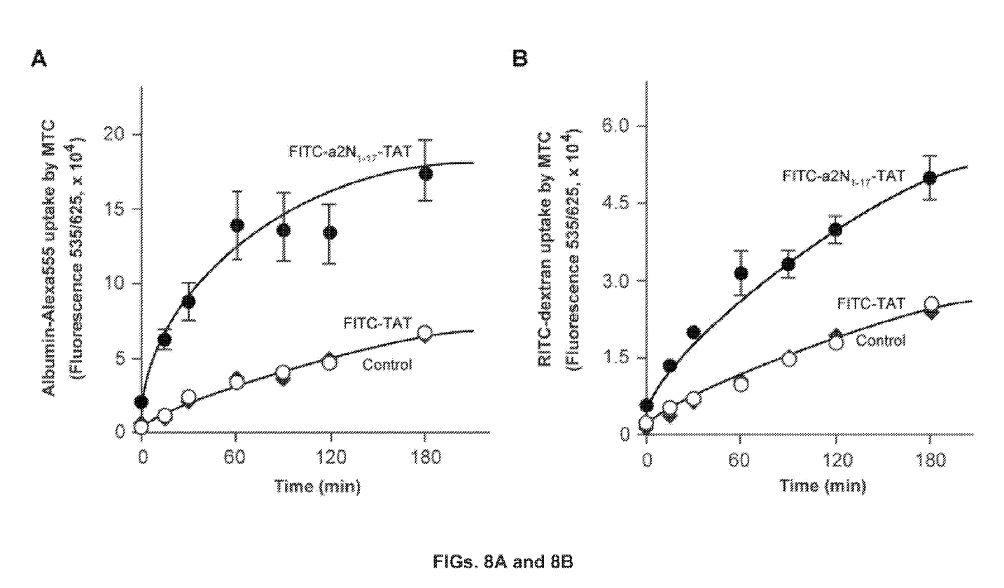

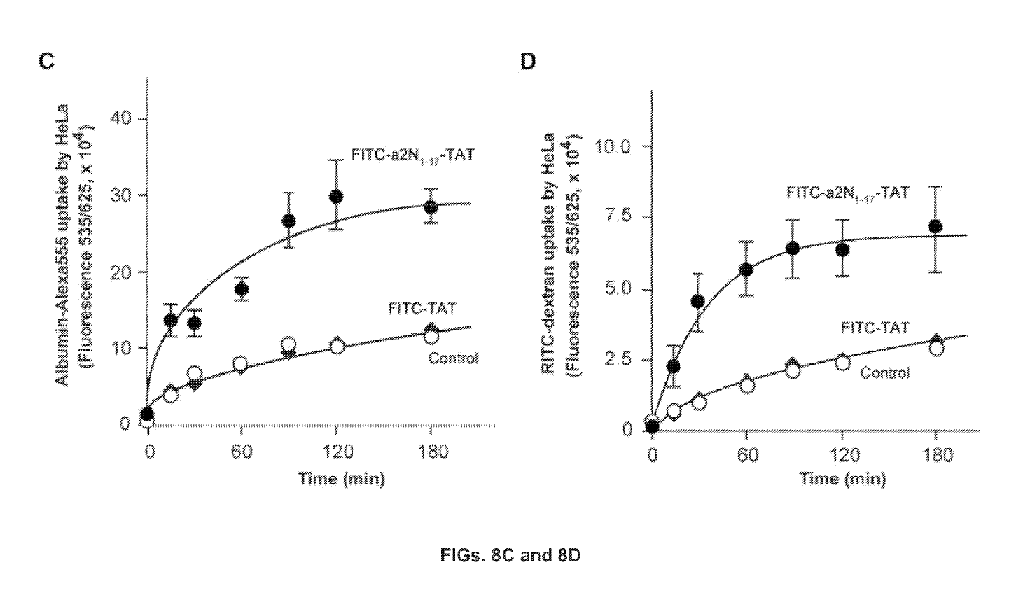

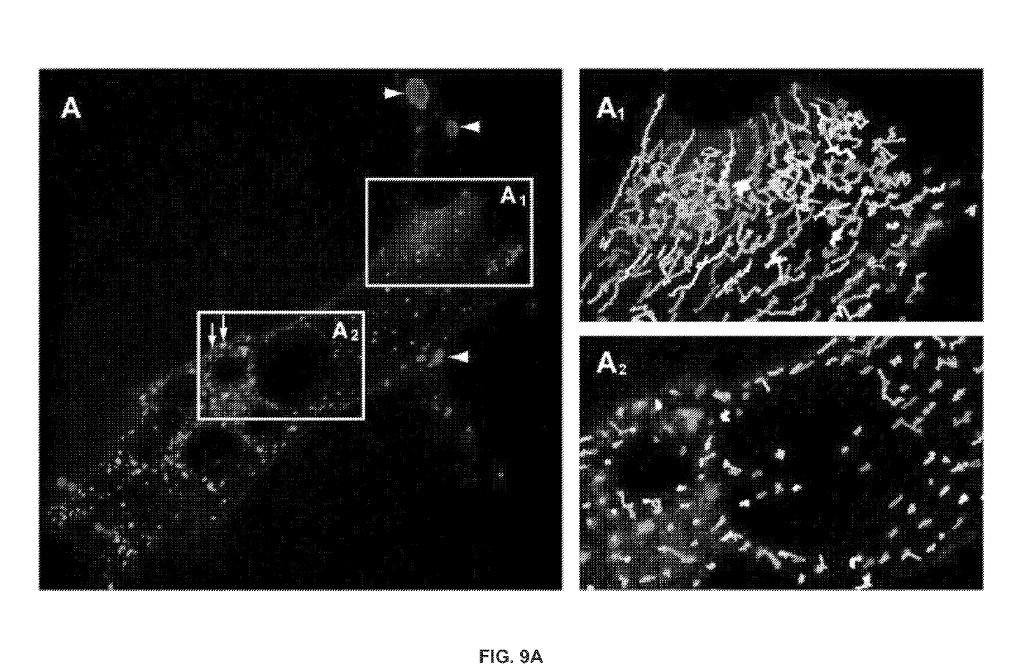

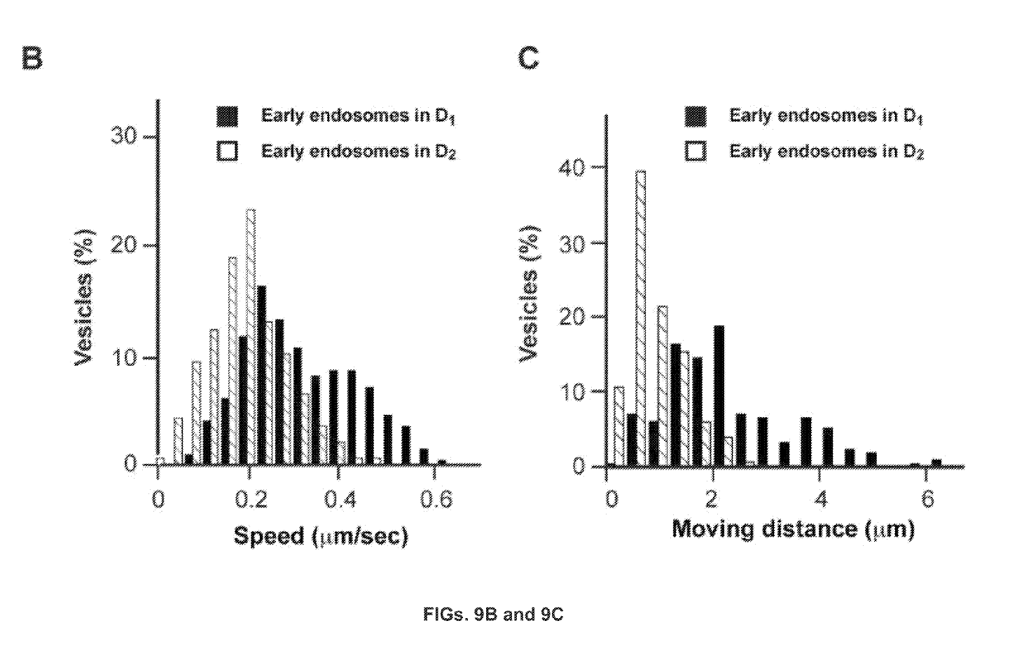

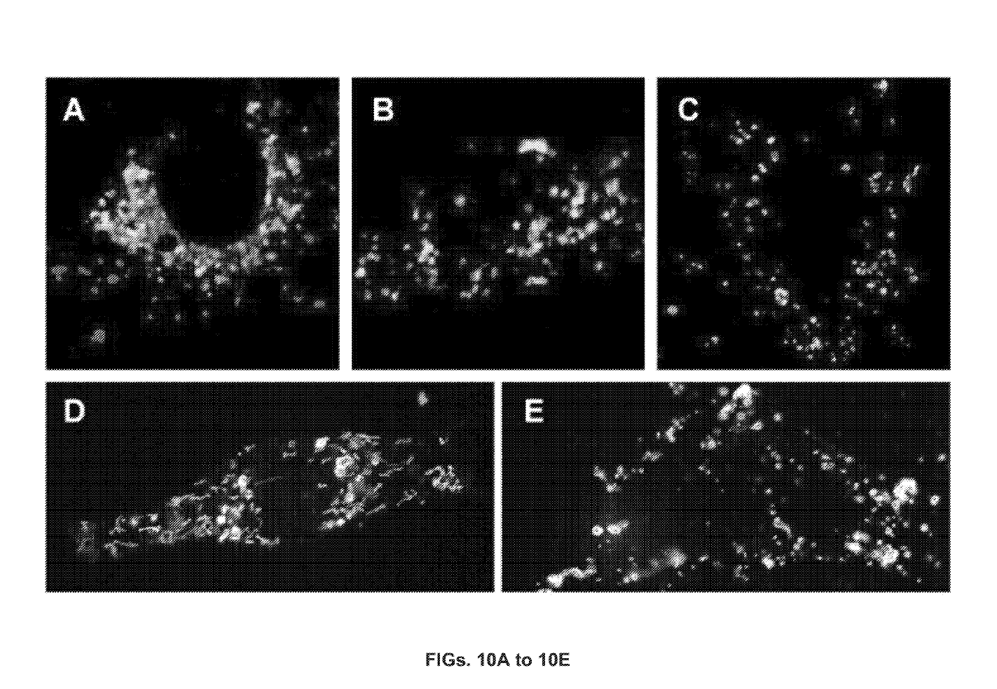

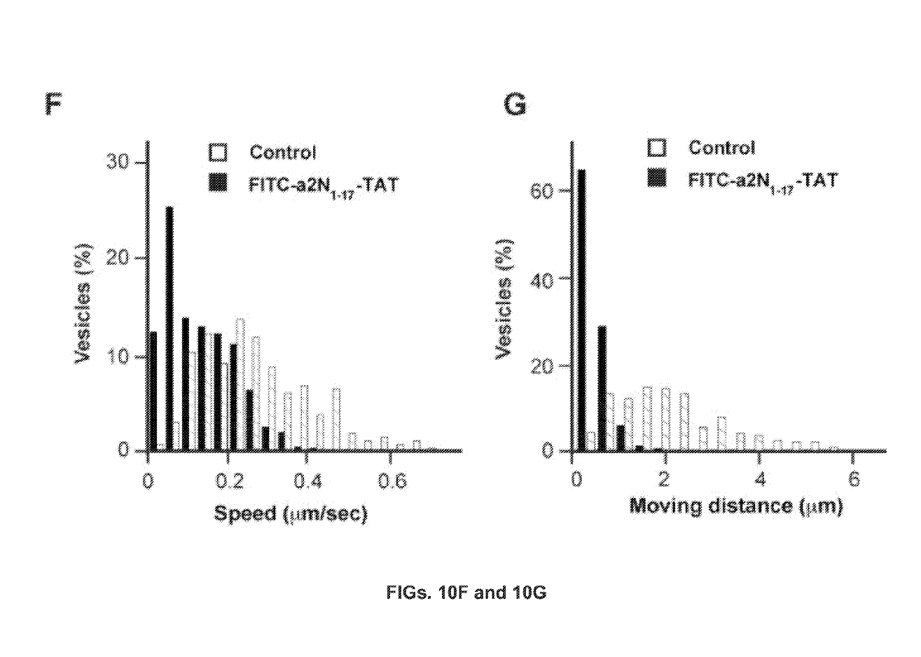

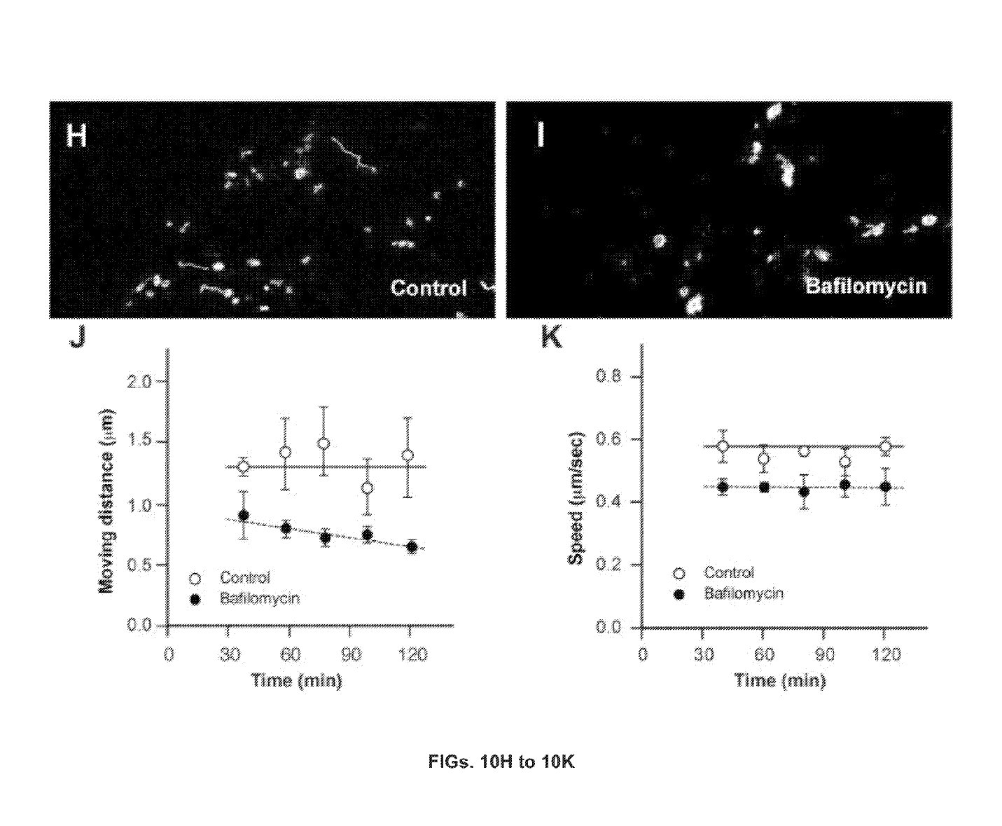

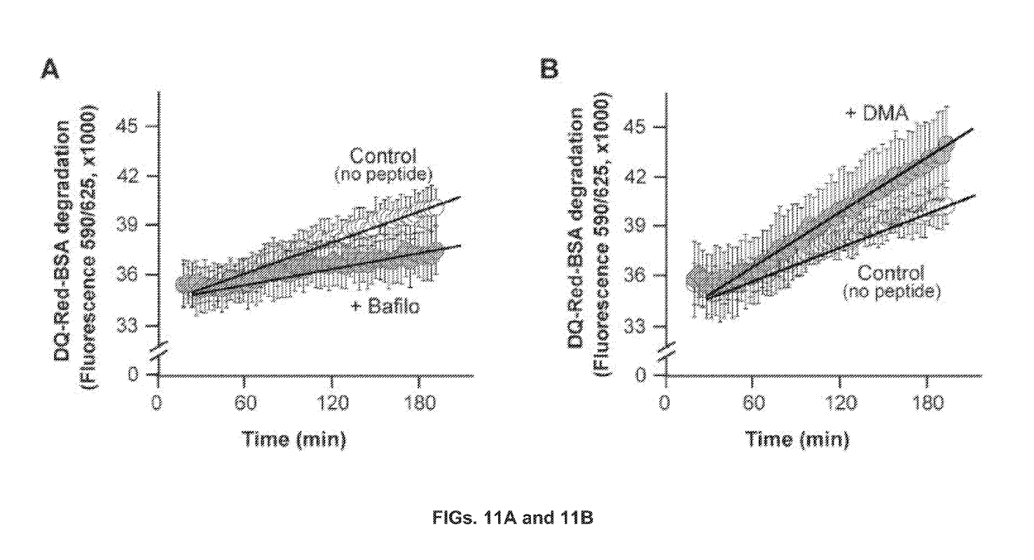

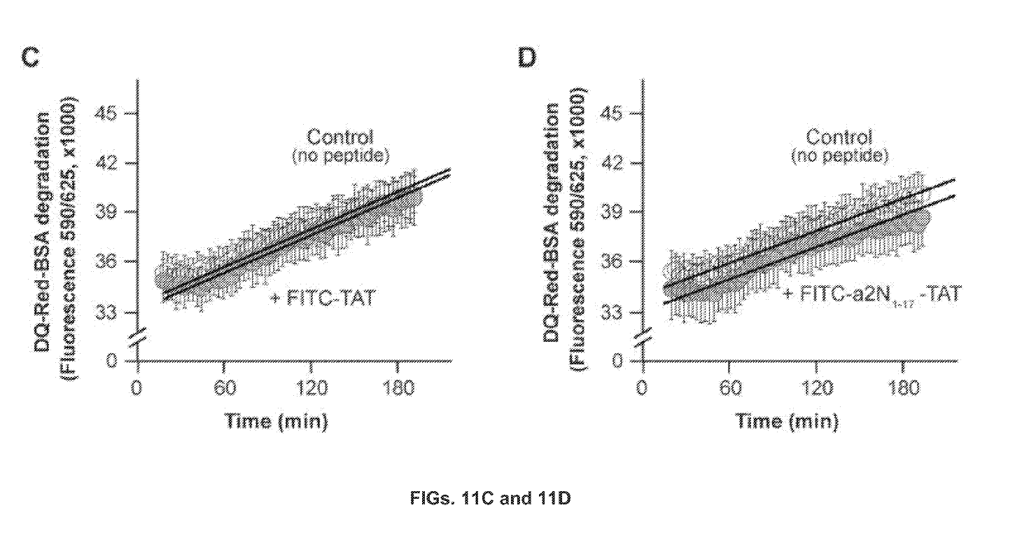

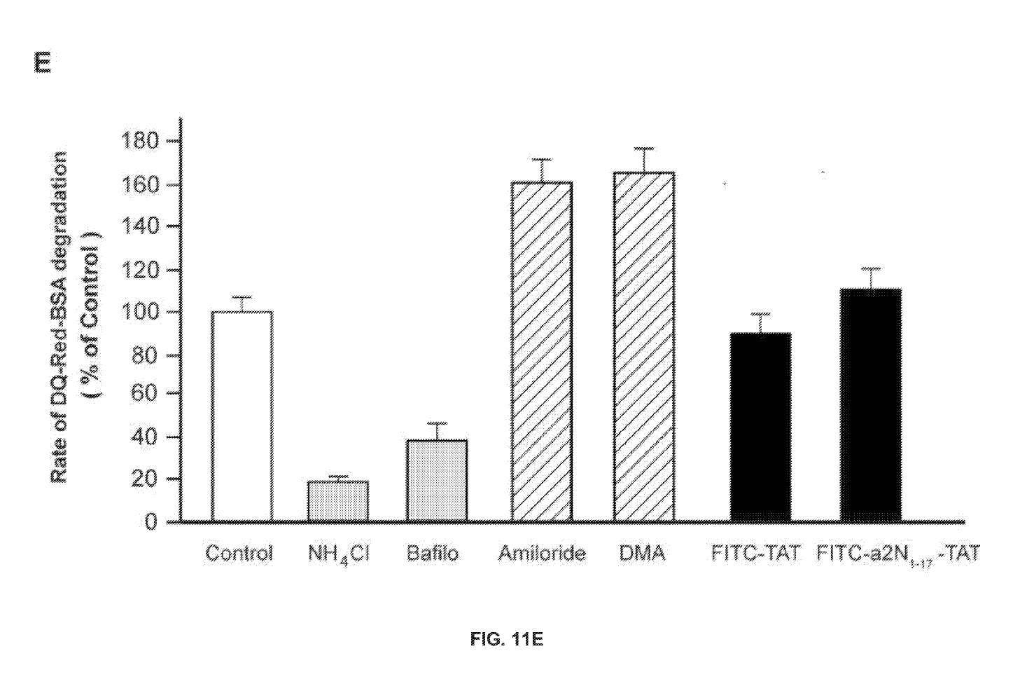

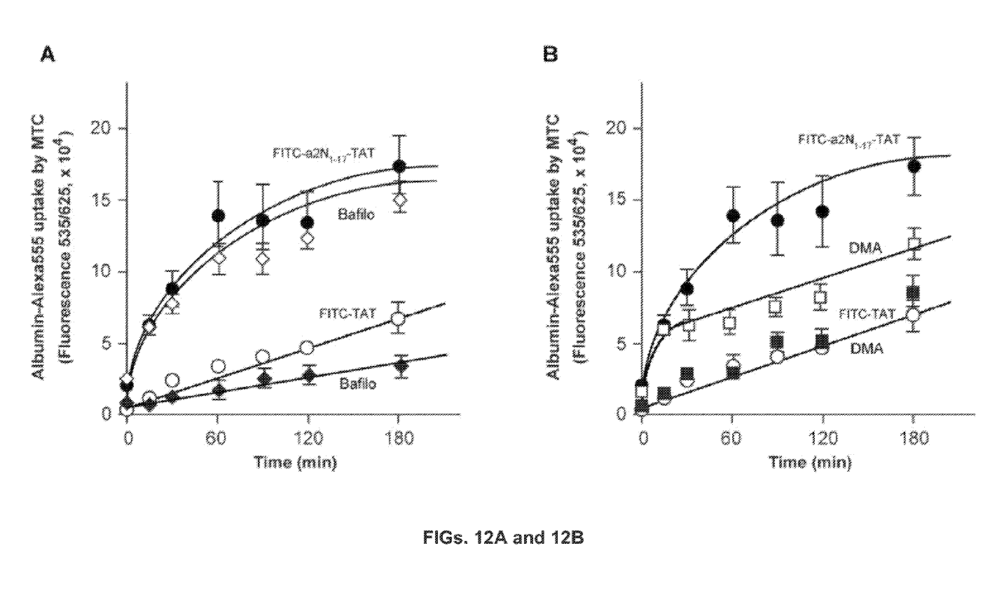

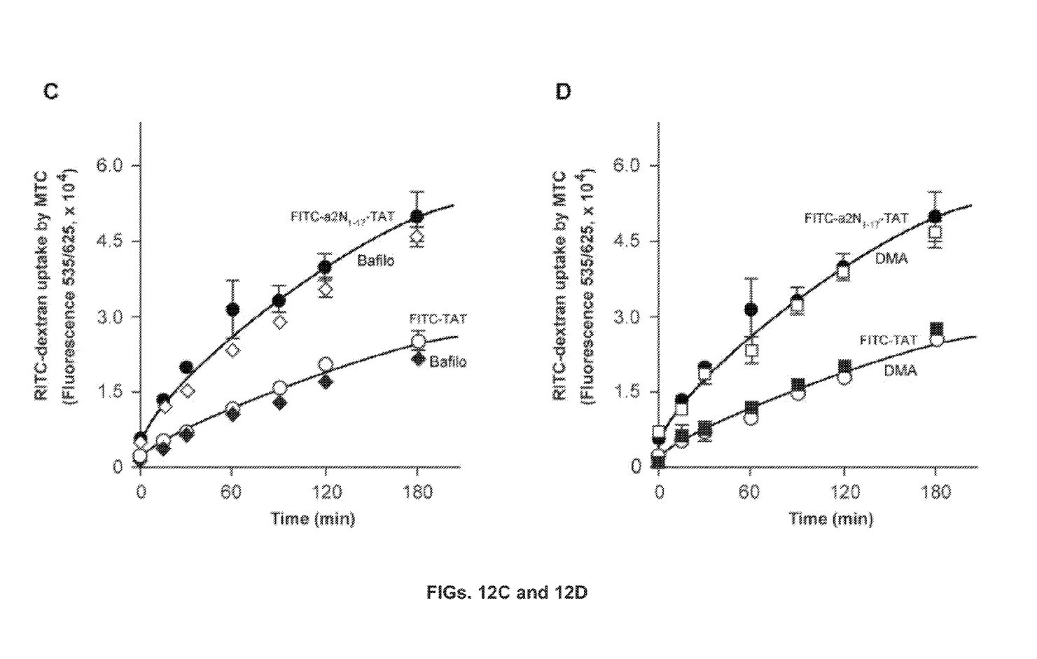

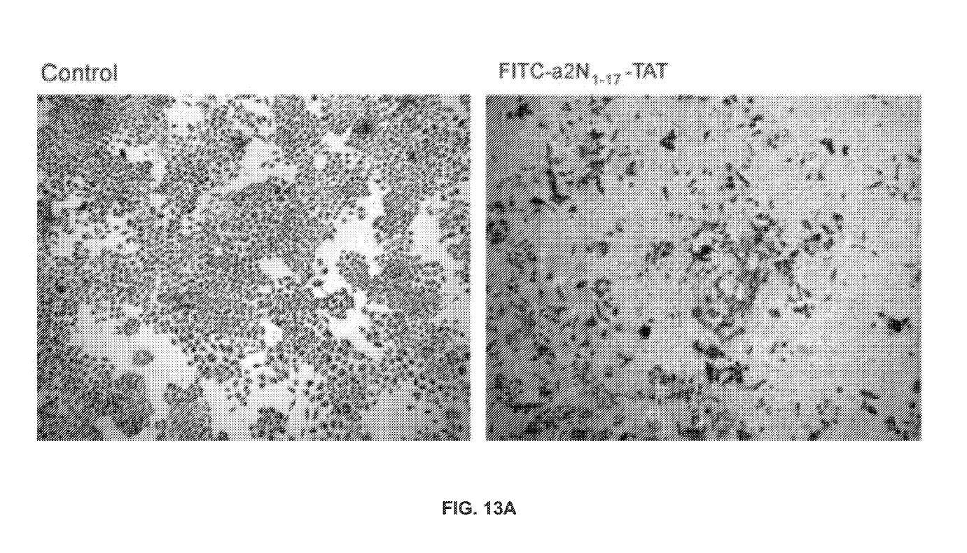

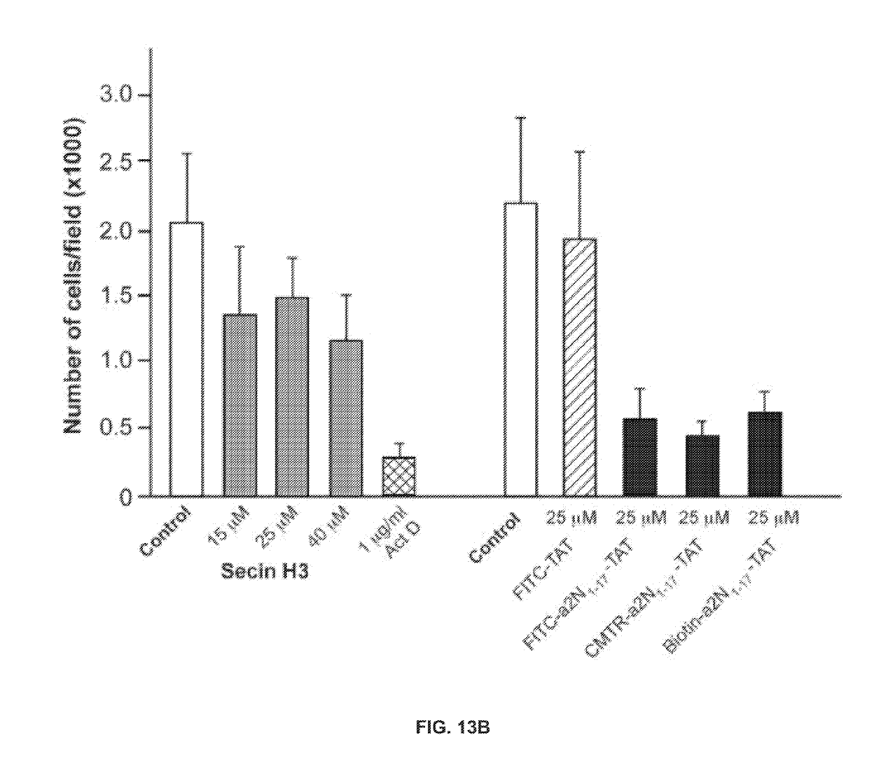

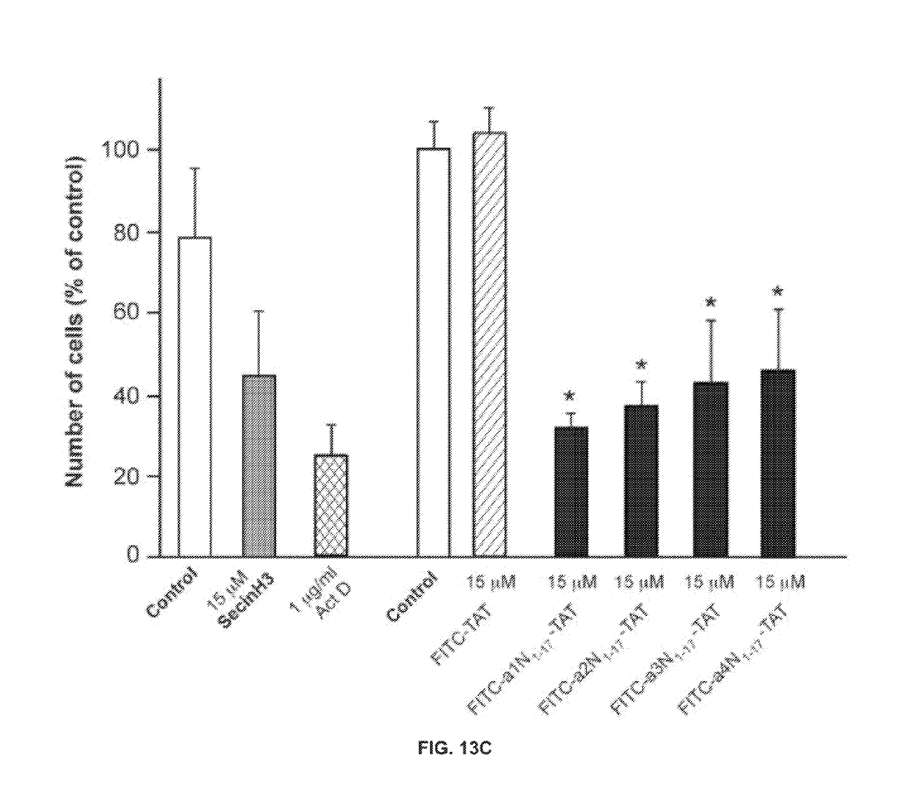

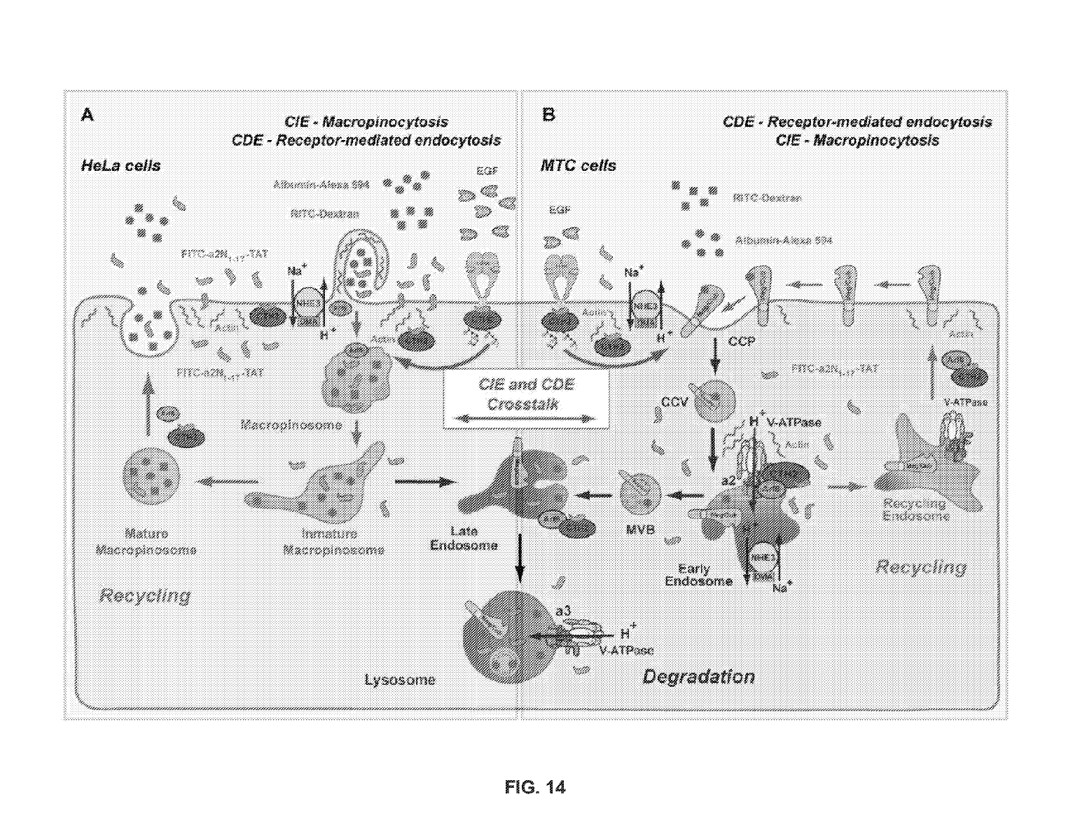

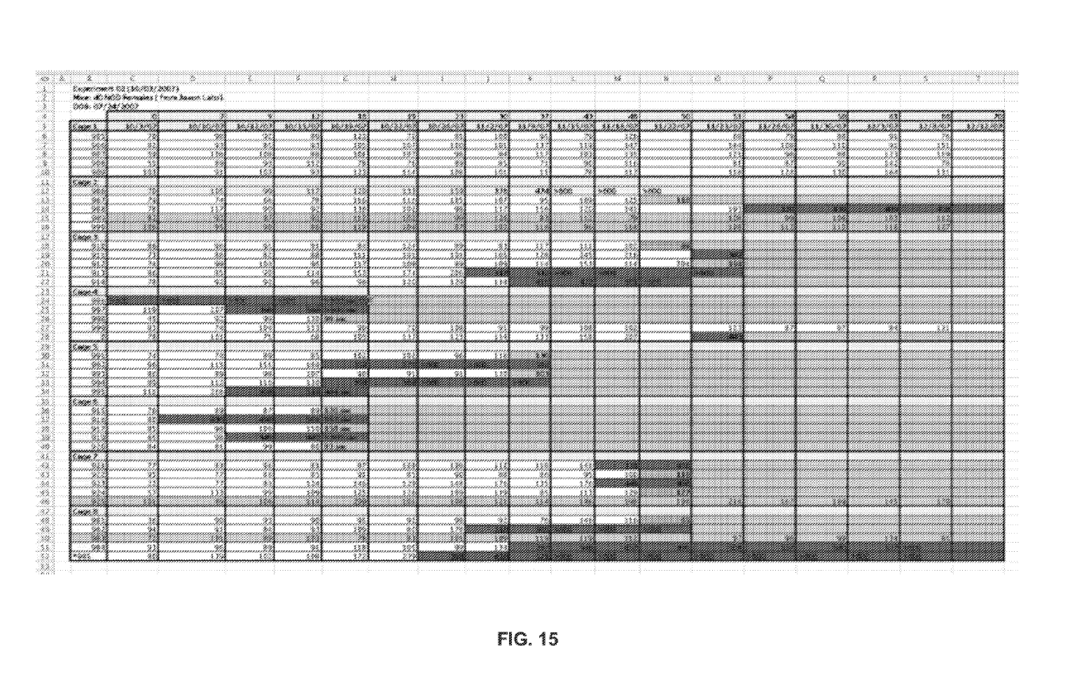

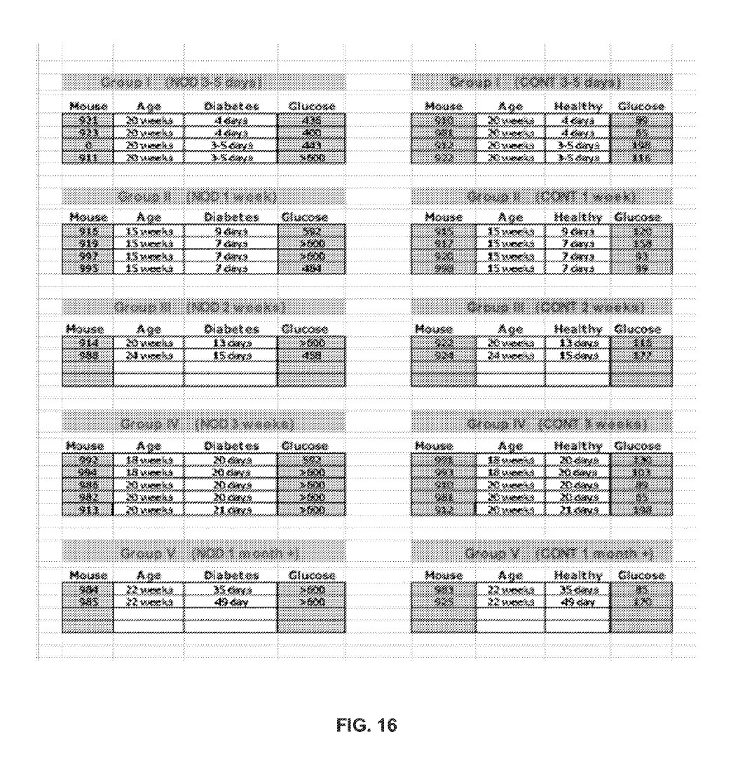

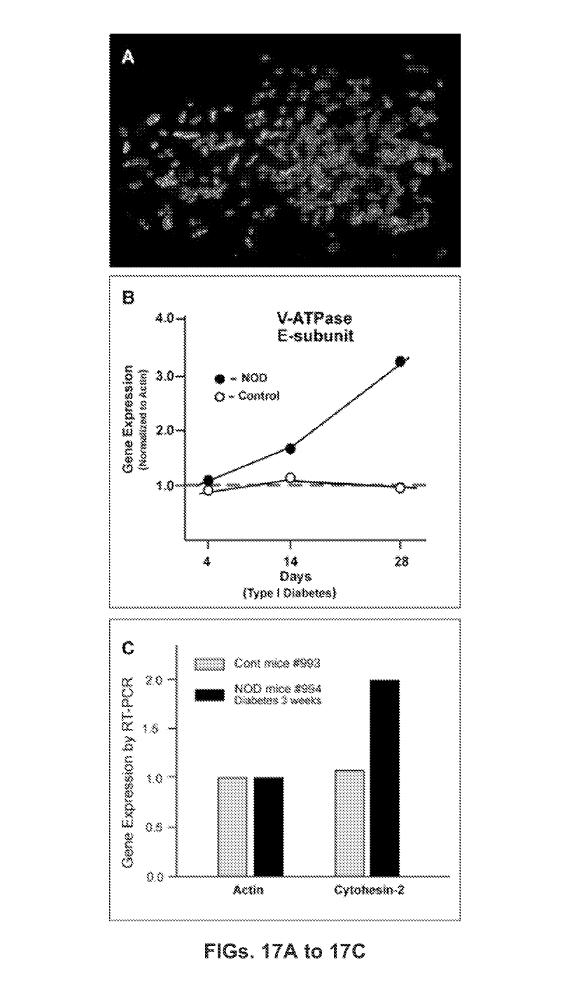

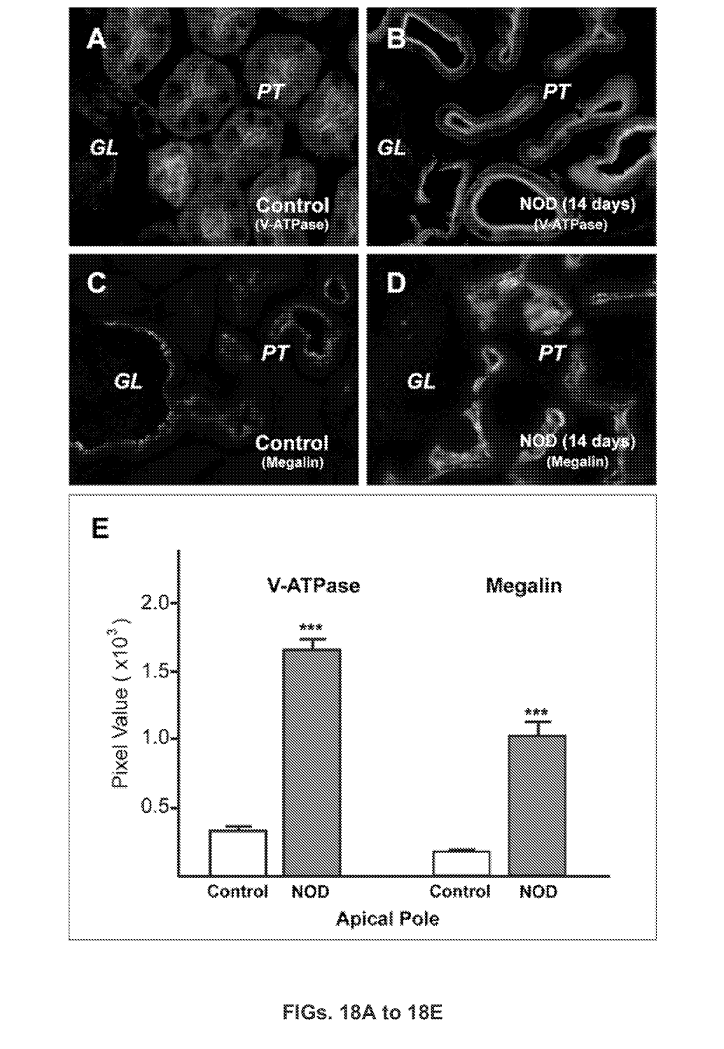

FIGS. 4B to 4D are line graphs showing potent inhibition of enzymatic GEF-activity of cytohesin-2 Sec7 domain by a1-, a3-, and a4-isoform derived peptides. FIG. 4B is a determination of IC₅₀=1.0 μM for FITC-a1N₁₋₁₇-TAT peptide. FIG. 4C is a determination of IC₅₀=0.5 μM for FITC-a3N₁₋₁₇-TAT peptide. FIG. 4D is a determination of IC₅₀=1.1 μM for FITC-a4N₁₋₁₇-TAT peptide.

FIG. 5A is a panel of three photomicrographs showing that in an kidney experimental system the V-ATPase complex remains intact and competent of acidification, interaction, and signaling with cytohein-2 and Arf GTP-binding proteins.

FIGS. 5B and 5C are photomicrographs showing that overexpressed recombinant full-length a2-EGFP-subunit isoform of V-ATPase remains intact (FIG. 5B) and competent to be targeted to vesicular compartment in kidney epithelial cells in culture (FIG. 5C). Scale bar, 4 μm.

FIG. 5D is a 3D map showing a holo-complex of Saccharomyces cerevisiae eukaryotic V-ATPase solved at 11 Å resolution (Benlekbir et al. (Nat Struct Mol Biol, In Press, 2012)). Scale bar, 25 Å.

FIG. 5E is a photomicrograph of an isolation of S. cerevisiae V-ATPase by elution from the M2-sepharose with the 3×FLAG peptide and characterization by SDS-PAGE analysis.

FIGS. 5F and 5G are photomicrographs showing that interaction of intact yeast V-ATPase holo-complex with mammalian cytohesin-2 is evolutionarily conserved. Yeast V-ATPase was immobilized on M2-sepharose allowed to interact with CTH2₆₁₋₄₀₀ and purified by SDS-PAGE (FIG. 5F) and Western blot analysis (FIG. 5G), demonstrated binding of mammalian CTH2₆₁₋₄₀₀ and its elution with intact yeast V-ATPase holo-complex.

FIG. 6 shows signaling of V-ATPase with cytohesin-2 and ArfGTP-binding proteins are evolutionarily conserved among all four a-subunit isoforms of the V-ATPase. Experimental IC₅₀ values of GEF-activity inhibition of the cytohesin-2 Sec7 domain by a-subunit isoforms specific peptides are listed for each isoform.

FIGS. 7A to 7C are a panel of photomicrographs and a line graph showing that the cell permeable anti-cytohesin-2 peptide accumulated over time in MTC cells. FIGS. 7A and 7C show the V-ATPase derived FITC-a2N₁₋₁₇-TAT peptide is targeted to and accumulates over time in specific vesicular compartments in MTC cells. FIGS. 7B and 7C show the inactive control FITC-TAT peptide has a cytosolic distribution and does not accumulate in MTC cells.

FIGS. 8A and 8B are two line graphs showing that the V-ATPase-derived biologically active anti-cytohesin-2 peptide FITC-a2N₁₋₁₇-TAT, but not control peptide FITC-TAT potently activates endocytic uptake of both albumin-Alexa555 and RITC-dextran in MTC cells.

FIGS. 8C and 8D are two line graphs showing that the V-ATPase-derived biologically active anti-cytohesin-2 peptide FITC-a2N₁₋₁₇-TAT, but not control peptide FITC-TAT potently activates endocytic uptake of both albumin-Alexa555 and RITC-dextran in HeLa cells.

FIGS. 9A, 9A₁, and 9A₂ are a panel of photomicrographs showing the action of V-ATPase-derived anti-cytohesin-2 peptide on early stage of endocytic pathway.

FIG. 9B is a bar graph showing a quantitative analysis of early endosomal movement speed in the presence of (FIG. 9A₁) or without (FIG. 9A₂) cytosolic CTMR-a2N₁₋₁₇-TAT peptide.

FIG. 9C is a bar graph showing a quantitative analysis of early endosomal moving distance in the presence of (FIG. 9A₁) or without (FIG. 9A₂) cytosolic CTMR-a2N₁₋₁₇-TAT peptide.

FIGS. 10A and 10B are two photomicrographs showing effects of CTMR-a2N₁₋₁₇-TAT on late events of vesicular trafficking and function in the endosomal/lysosomal protein degradation pathway in MTC cells transiently transfected with a vector expressing Rab7-EGFP (FIG. 10A) or LAMP1-EGFP (FIG. 10B) markers of late endosomes and lysosomes, respectively.

FIG. 10C is a photomicrograph taken from a pulse-chase experiment with albumin-Alexa594 and FITC-a2N₁₋₁₇-TAT peptide pulsed for 10 min and chased for 2 hours. The figure is a frame of the corresponding movie.

FIGS. 10D and 10E are two photomicrographs showing uptake of albumin-Alexa555 for 10 min without (FIG. 10D) or with (FIG. 10E) FITC-a2N₁₋₁₇-TAT peptide followed by washing and immediate time-lapse imaging.

FIGS. 10F and 10G are two bar graphs depicting a quantitative analysis of late endosomal/lysosomal moving speed (FIG. 10F) and distance (FIG. 10G) in absence or presence of FITC-a2N₁₋₁₇-TAT peptide.

FIGS. 10H and 10I are two photomicrographs taken from a pulse-chase experiment with albumin-Alexa594 without (FIG. 10H) or with (FIG. 10I) bafilomycin A₁ treatment.

FIGS. 10J and 10K are two line graphs depicting a quantitative analysis of late endosomal/lysosomal moving distance (FIG. 10J) and speed (FIG. 10K) in absence or presence of bafilomycin A₁.

FIGS. 11A to 11E are a panel of graphs showing differential action of specific inhibitors of V-ATPase, Na⁺/H⁺-exchanger, and anti-cytohesin-2 peptide on degradation of DQ-Red-BSA in MTC cells. FIGS. 11A and 11E show that the V-ATPase inhibitor bafilomycin A₁ and uncoupler NH₄Cl strongly inhibited endosomal/lysosomal degradation of DQ-Red-BSA. FIGS. 11B and 11E show that the NHE-inhibitor DMA increased degradation of DQ-Red-BSA in endosomal/lysosomal compartments of MTC cells. FIGS. 11C, 11D, and 11E show that both control FITC-TAT and FITC-a2N₁₋₁₇-TAT peptides do not affect degradation of DQ-Red-BSA in endosomal/lysosomal compartments of MTC cells.

FIG. 12A is a line graph showing that the V-ATPase inhibitor bafilomycin A₁ inhibits uptake of albumin-Alexa555 via receptor-mediated CDE pathway, but does not modulate an uptake of albumin-Alexa555 via FITC-a2N₁₋₁₇-TAT peptide-induced macropinocytosis CIE pathway.

FIG. 12A is a line graph showing that the NHE-inhibitor, DMA does not modulate an uptake of albumin-Alexa555 via receptor-mediated CDE pathway but strongly inhibits the FITC-a2N₁₋₁₇-TAT peptide-induced uptake of albumin-Alexa555 via macropinocytosis CIE pathway.

FIGS. 12C and 12D are two line graphs showing that an endocytic uptake of fluid phase marker RITC-dextran is strongly activated by anti-cytohesin-2 peptide and is not modulated by the bafilomycin A₁ (FIG. 12C) or DMA (FIG. 12D), inhibitors of V-ATPase and Na⁺/H⁺-exchanger, respectively.

FIG. 13A is a panel of two photomicrographs showing that the V-ATPase derived cell permeable anti-cytohesin-2 FITC-a2N₁₋₁₇-TAT peptide is a potent inhibitor of human A549 lung cancer cells proliferation in vitro.

FIG. 13B is a bar graph comparing anti-proliferative action of FITC-, CMTR-, Biotin-a2N₁₇-TAT anti-cytohesin-2 peptides with anti-cytohesin-2 small molecule inhibitor SecinH3.

FIG. 13C is a bar graph comparing anti-proliferative action of V-ATPase a-subunits-specific anti-cytohesin-2 peptides including: i) FITC-a1N₁₇-TAT; ii) FITC-a2N₁₇-TAT; iii) FITC-a3N₁₇-TAT; and iv) FITC-a4N₁₇-TAT with anti-cytohesin-2 small molecule inhibitor SecinH3.

FIGS. 14A and 14B is a schematic drawing of the signaling between V-ATPase and cytohesin-2 as a regulatory switch between receptor-mediated and macropinocytosis endocytic pathways.

FIG. 15 is a table showing onset of type 1 diabetes in NOD mice, as determined by daily measurement of tail-blood glucose levels. Mice showing blood glucose levels higher than 300 mg/dL for two consecutive days were considered to be diabetic.

FIG. 16 is a panel of two tables with a summary of control and diabetic NOD mice colonies. Mice were separated into four diabetic NOD groups (0, 1, 2, 3, and more than 4 weeks) (left panel) and four control groups (0, 1, 2, 3, and more than 4 weeks) (right panel) with 2-4 mice in each group. The age, duration of diabetes and levels of glucose of each mice are indicated.

FIG. 17A is a photomicrograph of isolated mouse kidney proximal tubules (PT) by laser-capture microdisection (LCM).

FIGS. 17B and 17C are graphs showing levels of gene expression of V-ATPase (FIG. 17B) and cytohesin-2 (FIG. 17C) in mouse PTs during type 1 diabetes.

FIG. 18A to 18E is a panel of four photomicrographs and a bar graph showing levels of expression and distribution of V-ATPase (FIGS. 18A, 18B, and 18E) and megalin proteins (FIGS. 18C, 18D, and 18E) in kidney proximal tubules of control and NOD-mice during type 1 diabetes.

### DETAILED DESCRIPTION

Cell proliferation is universally controlled by the epidermal growth factor receptor EGFR/ErbB-family signaling pathway that is crucial in the development of cancer, e.g., lung cancer (Cohen (J Biol Chem 237, 1555-1562, 1962); Cohen et al. (J Biol Chem 255, 4834-4842, 1980); Normanno et al. (Gene 366, 2-16, 2006); Zhang et al. (J Clin Invest 117, 2051-2058, 2007); Sharma and Settleman (Exp Cell Res 315, 557-571, 2009)). Cytohesin-2 has recently emerged as an intracellular EGFR/ErbB-receptor activator that also controls cell proliferation and lung cancer development (Bill et al. (Cell 143, 201-211, 2010); Bill et al. (PLoS ONE 7, e41179, 2012)). It is noteworthy that one member of the EGFR/ErbB-receptor family, ErbB4, is also expressed in kidney PT epithelial cells and is involved in their polarization and proliferation (Veikkolainen et al. (J Am Soc Nephrol 23, 112-122, 2012); Zeng et al. (Mol Biol Cell 18, 4446-4456, 2007)). Importantly, the recent Genetics of Nephropathy and International Effort (GENIE) consortium Genome-Wide Association Study (GWAS) identified the ErbB4 gene as strongly associated with development of diabetic nephropathy (DN) caused by type 1 diabetes in humans (Sandholm et al. (PLoS Genet 8, e1002921, 2012); Boger and Sedor (PLoS Genet 8, e1002989, 2012)). However, its pathophysiological action in development of DN is currently unknown.

The present disclosure is based, in part, on the discovery that V-ATPase is a novel signaling receptor that interacts and potently modulates function of cytohesin-2 in the kidney PT endosomal/lysosomal pathway. Further, the disclosure is based, in part, on the discovery of a novel interaction of cytohesin-2 with aldolase. Aldolase can function as a “glucose sensor” and modulate gene expression and vesicular trafficking within the glycolytic pathway. Thus, these discoveries place cytohesin-2, V-ATPase, aldolase, and EGFR/ErbB-receptors, which all function within the endosomal/lysosomal pathway, in a unique position to control cell proliferation and vesicular trafficking. In particular, the central role of cytohesin-2 as a master controller of EGFR/ErbB/cytohesin-2/V-ATPase/aldolase and endosomal/lysosomal pathways in cancer cells, e.g., non-small cell lung cancer (NSCLC) cells, and kidney cells, e.g., proximal tubule (PT) epithelial cells, plays a crucial role in pathogenesis of diseases such as cancer, e.g., lung cancer, and diabetic kidney disease. Accordingly, the present disclosure provides methods of inhibiting cytohesin-2 to treat cancer, to treat DN, and to deliver a compound into a cell.

### Aldolase: Cytohesin-2 Related Function of Glycolytic Enzyme

The present disclosure is based, at least in part, on the discovery that aldolase modulates the biological function of cytohesin-2. Fructose bisphosphate aldolase (ALDO) (EC 4.1.2.13) is a critical enzyme of glycolysis, which catalyzes the reversible cleavage of fructose 1,6-bisphosphate into glyceraldehyde 3-phosphate and dihydroxyacetone phosphate. While the role of aldolase in carbohydrate metabolism is well established, there is growing evidence for many alternative so-called “moonlighting” functions for this enzyme. In particular, aldolase interacts with various proteins, including: i) F-actin; ii) α-tubulin; iii) light chain 8 of dynein; iv) actin nucleation promoting factor WASP; vi) endocytotic sorting protein nexin-9; and vii) phospholipase D₂ (Wang et al. (J Biol Chem 271, 6861-6865, 1996); Wang et al. (Exp Cell Res 237, 445-451, 1997); Buscaglia et al. (J Biol Chem 281, 1324-1331, 2006); Kim et al. (Biochemistry 41, 3414-3421, 2002)). These multiple interactions indicate that aldolase is probably involved as a scaffolder, in coordinating membrane trafficking and cytoskeleton dynamics. As a result, a novel emerging field for aldolase biology is its central role in a variety of vesicular trafficking events including: i) endocytosis; ii) cytoskeleton rearrangement and cell motility; iii) trafficking and recycling of membrane proteins; and iv) signal transduction. In addition, the interaction between aldolase and the V-ATPase was also documented (Lu et al. (J Biol Chem 282, 24495-24503, 2007); Lu et al. (J Biol Chem 276, 30407-30413, 2001); Lu et al. (J Biol Chem 279, 8732-8739, 2004)). Aldolase interacts with three different subunits of the V-ATPase: the transmembrane a-subunit of the Vₒ-sector; as well as the soluble E- and B-subunits of the V₁-sector. The interaction between aldolase and the V-ATPase is modified by glucose, suggesting that aldolase may act as a “glucose sensor” for which physical association with V-ATPase is important for its assembly and function (Lu et al. (J Biol Chem 282, 24495-24503, 2007)). Aldolase functions as a “glucose sensor” through interaction with cytohesin-2 and V-ATPase to play an important role in regulation of endosomal/lysosomal pathway in proximal tubules during early stages of DN.

### EGFR/ErbB-Receptors: Cytohesin-2 is a Novel EGFR/ErbB-Receptors Activator and Drug Target

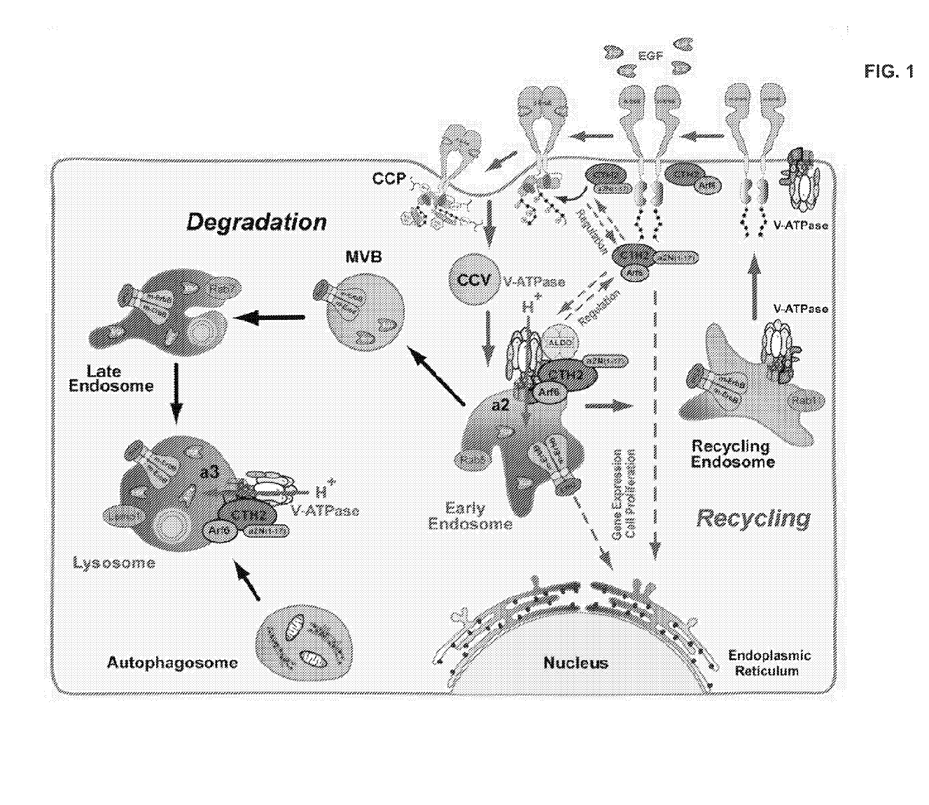

Until recently, the cytoplasmic proteins that are able to directly modulate EGF-induced activation and signaling of EGFR/ErbB-receptors were largely unknown. However, recently cytohesin-2 was identified as a crucial cytoplasmic EGFR/ErbB-receptor activator (Bill et al. (Cell 143, 201-211, 2010)). The epidermal growth factor receptor (EGFR) was among the first discovered signaling receptors that regulate crucial cell biological processes including cell proliferation (Cohen (J Biol Chem 237, 1555-1562, 1962); Cohen et al. (J Biol Chem 255, 4834-4842, 1980); Normanno et al. (Gene 366, 2-16, 2006); Zhang et al. (J Clin Invest 117, 2051-2058, 2007); Sharma and Settleman (Exp Cell Res 315, 557-571, 2009)). The EGFR/ErbB receptor family comprises four members including: i) EGFR; ii) ErbB-2; iii) ErbB-3; and iv) ErbB-4. These receptors are composed of five domains including: i) extracellular domain; ii) transmembrane domain; iii) juxtamembrane domain; iv) tyrosine kinase (TK) domain; and v) C-terminal tail (Ferguson (Annu Rev Biophys 37, 353-373, 2008)). Activation of EGFR/ErbB-receptors by EGF among other ligands promotes their hetero-dimerization with subsequent activation of TK domains and tyrosine trans-phosphorylation of the cytoplasmic tail. Activation of these receptors is signaling through diverse intracellular pathways that modulate c-fos, c-Jun and c-myc transcription factors and cell proliferation (Normanno et al. (Gene 366, 2-16, 2006); Zhang et al. (J Clin Invest 117, 2051-2058, 2007); Ferguson (Annu Rev Biophys 37, 353-373, 2008)). Cytohesin-2 enhances activation of EGFR ErbB-receptors by direct binding with TK-domains of dimerized receptors and by facilitating conformational changes and trans-phosphorylation of these domains. Importantly, the function and signaling of EGFR/ErbB-receptors are also modulated by their trafficking in the endosomal/lysosomal pathway via their recycling or degradation. It is noteworthy that the function and signaling of EGFR/ErbB-receptors in early endosomes is pivotal for their fate in recycling or degradation. V-ATPase, cytohesin-2, and aldolase play a crucial role in this pathway (Hurtado-Lorenzo et al. (Nat Cell Biol 8, 124-136, 2006); Hosokawa et al. (J Biol Chem 288:5896-5913, 2013); Merkulova et al. (Am J Physiol Cell Physiol 300, C1442-1455, 2011); Marshansky and Futai (Curr Opin Cell Biol 20, 415-426, 2008); Marshansky (Biochem Soc Trans 35, 1092-1099, 2007)). Thus, the endosomal/lysosomal-dependent function of EGFR/ErbB/cytohesin-2/V-ATPase/aldolase-pathway is in a unique position to participate in the pathogenesis of a variety of diseases including cancer and diabetes (FIG. 1). In particular, the cytohesin-2-dependent signaling of EGFR/ErbB-receptors is recognized as key signaling event in the development of a variety of human tumors, including NSCLC (Cohen (J Biol Chem 237, 1555-1562, 1962); Cohen et al. (J Biol Chem 255, 4834-4842, 1980); Normanno et al. (Gene 366, 2-16, 2006); Zhang et al. (J Clin Invest 117, 2051-2058, 2007); Sharma and Settleman (Exp Cell Res 315, 557-571, 2009)). Taking into consideration the importance of EGFR/ErbB-receptors in pathogenesis of cancer they are considered as important anti-cancer drug targets. Indeed, over the past ten years, various therapeutic agents that target EGFR/ErbB receptors have been FDA approved or are currently in clinical trials. In particular, two major classes of anti-EGFR/ErbB therapeutics include: i) monoclonal antibodies (e.g., cetuximab, panitumumab) that target extracellular ligand binding domain; and ii) small molecule tyrosine kinase inhibitors (TKI) (e.g., gefitinib, erlotinib, lapatinib) that target cytoplasmic TK domains. They have shown significant benefit in treatment of lung cancer among other tumors (Baselga and Arteaga (J Clin Oncol 23, 2445-2459, 2005)). However, a large population of patients with NSCLC does not respond to TKI treatment. Thus, it is imperative to understand the causes of resistance and to uncover new therapeutic targets within the EGFR/ErbB/cytohesin-2 signaling pathway in order to overcome the current therapeutic limitations, in particular for treatment of TKI-resistant cancers.

Cytohesin-2 has emerged as such a novel anti-proliferative drug target useful for treatment of cancer, e.g., lung cancer, including TKI-resistant cancers. In particular, an anti-cytohesin-2 small molecule called SecinH3 was uncovered in high-throughput screening and tested in lung cancer cells and animal models (Bill et al. (Cell 143, 201-211, 2010); Bill et al. (PLoS ONE 7, e41179, 2012)). These studies demonstrated that targeting of cytohesin-2 by SecinH3 or its knock-down give rise to: i) reduction of EGFR/ErbB-signaling; ii) suppression of cancer cell proliferation in vitro; and iii) reduction of growth of tumor xenografts in nude mice model in vivo (Bill et al. (Cell 143, 201-211, 2010); Bill et al. (PLoS ONE 7, e41179, 2012)). In contrast, as described herein, eight V-ATPase-derived peptides that are more than 100-fold more potent inhibitors of cytohesin-2 than SecinH3 have been identified and characterized. It is important to underline, that these cell-permeable peptides are also very potent inhibitors of cell proliferation, including TKI-resistant lung cancer 549 cells. These peptides and drugs may be useful for treatment of cancers (including TKI-resistant cancers) and reducing a risk of developing diabetic nephropathy during type 1 diabetes.

### Vacuolar-Type H⁺-ATPase (V-ATPase)

The vacuolar-type H⁺-ATPase (V-ATPase) is a proton pumping nano-motor involved in acidification of variety intracellular organelles and extracellular compartments. It is absolutely essential for vesicular trafficking along both exocytotic and endocytotic pathways of eukaryotic cells. Deficient function of V-ATPase and defects of vesicular acidification have been also recently recognized as important mechanisms of variety of human diseases and discussed below. Consistent with the presence of V-ATPases in diverse compartments a large spectrum of subunit isoforms were found (Aridor and Hannan Traffic 2000, 1:836-851; Aridor and Hannan Traffic 2002, 3:781-790; Marshansky and Futai, Curr Opin Cell Biol 2008, 20:415-426; Grüeber and Marshansky, BioEssays 2008, 30: 1096-1099; Hurtado-Lorenzo et al., Nature Cell Biol 2006, 6:124-136; Forgac, Nat. Rev. Mol. Cell Biol. 2007, 11: 917-29; Hinton et al., Pflugers Arch. 2009, 457: 589-598). The expression of these isoforms is tissue and cell specific. Recent experiments demonstrated that targeting of V-ATPases with unique combinations of subunit isoforms are localized in specific cellular membranes which could be dictated by their functions (Wagner et al., Physiol. Rev. 2004, 84:1263-1314; Marshansky and Futai, Curr Opin Cell Biol 2008, 20:415-426; Grüeber and Marshansky, BioEssays 2008, 30: 1096-1099). Intracellular targeting of V-ATPase is regulated by a-subunit isoforms. Four a-subunit isoforms (a1, a2, a3, and a4) were found in mice and humans (Toyomura et al., J Biol Chem 2000, 275: 8760-8765; Oka et al., J Biol Chem 2001, 276: 40050-40054; Smith et al., J Biol Chem 2001, 276: 42382-42388; Nishi and Forgac, J Biol Chem 2000, 275: 6824-6830). They are localized in different endomembrane organelles and plasma membrane of specialized cells (Wagner et al., Physiol. Rev. 2004, 84:1263-1314; Marshansky and Futai, Curr Opin Cell Biol 2008, 20:415-426; Grüeber and Marshansky, BioEssays 2008, 30: 1096-1099).

The sequences of mouse a1-subunit V ATPase are available in the GenBank database under Accession Nos. NP_{—}058616.1 (amino acid), NM_{—}016920.3 (nucleic acid); NP_{—}001229979.1 (amino acid), NM_{—}001243050.1 (nucleic acid); and NP_{—}001229980.1 (amino acid), NM_{—}001243051.1 (nucleic acid). The sequences of mouse a2-subunit V ATPase is available in the GenBank database under Accession Nos. NP_{—}035726.2 (amino acid), NM_{—}011596.4 (nucleic acid); and NP_{—}035726.2 (amino acid), NM_{—}011596.4 (nucleic acid). The sequences of mouse a3-subunit V ATPase are available in the GenBank database under Accession Nos. NP_{—}001129563.1 (amino acid), NM_{—}001136091.2 (nucleic acid). The sequences of mouse a4-subunit V ATPase are available in the GenBank database under Accession Nos. NP_{—}536715.3 (amino acid), NM_{—}080467.3 (nucleic acid).

The sequences of human a1-subunit V ATPase are available in the GenBank database under Accession Nos. NP_{—}001123492.1 (amino acid), NM_{—}001130020.1 (nucleic acid). The sequences of human a2-subunit V ATPase are available in the GenBank database under Accession Nos. NP_{—}036595.2 (amino acid), NM_{—}012463.3 (nucleic acid). The sequences of human a3-subunit V ATPase are available in the GenBank database under Accession Nos. NP_{—}006010.2 (amino acid), NM_{—}006019.3 (nucleic acid). The sequences of human a4-subunit V ATPase are available in the GenBank database under Accession Nos. NP_{—}065683.2 (amino acid), NM_{—}020632.2 (nucleic acid).

### aN Peptides

Described herein are aN-01 or aN (1-17) peptides shown in Table 1 and aN-03 or aN (35-49) peptides shown in Table 2 of four isoforms of V-ATPase a-subunits. In human and mice, the following V-ATPase subunit isoforms are found: i) two isoforms for the B, E, H, and d-subunits; ii) three isoforms for the C and G-subunits; and iii) four isoforms for a-subunit (a1, a2, a3, and a4). The expression and targeting of all these isoforms is cell- and organelle-specific. Importantly, targeting and localization of V-ATPase in different compartments along both endocytic and exocytic pathways as well as plasma membrane depends upon a-subunits isoforms (Marshansky and Futai (Curr Opin Cell Biol 20, 415-426, 2008)). In mammalian cells, targeting and compartment specific localization of V-ATPase depends on a-subunit isoforms.

TABLE 1
Mouse and Human V-ATPase a-subunit isoforms derived
Peptides aN-01 or aN(1-17)

| | | |
|---|---|---|
| | | SEQ |
| Name | Label & Sequence | ID NO: |

Mus musculus
a1-subunit V-ATPase, (Mus musculus: NP_058616.1, NM_016920.3;
NP_001229979.1, NM_001243050.1; NP_001229980.1, NM_001243051.1)

| | | |
|---|---|---|
| a1N-01 or | MGELFRSEEMTLAQLFL | 1 |
| a1N(1-17) | | |
| Biotin- a1N(1-17) | Biotin - MGELFRSEEMTLAQLFL | 1 |
| a1N(1-17)-TAT | MGELFRSEEMTLAQLFL - YGRKKRRQRRR | 2 |
| Biotin- a1N(1-17)-TAT | Biotin- MGELFRSEEMTLAQLFL - YGRKKRRQRRR | 2 |
| Fluor- a1N(1-17)-TAT | Fluor- MGELFRSEEMTLAQLFL - YGRKKRRQRRR | 2 |
| FITC- a1N(1-17)-TAT | FITC- MGELFRSEEMTLAQLFL - YGRKKRRQRRR | 2 |
| CMTR- a1N(1-17)-TAT | CMTR- MGELFRSEEMTLAQLFL - YGRKKRRQRRR | 2 |

a2-subunit V-ATPase, (Mus musculus: NP_035726.2, NM_011596.4;
NP_035726.2, NM_011596.4)

| | | |
|---|---|---|
| a2N-01 or | MGSLFRSESMCLAQLFL | 3 |
| a2N(1-17) | | |
| Biotin- a2N(1-17) | Biotin- MGSLFRSESMCLAQLFL | 3 |
| a2N(1-17)-TAT | MGSLFRSESMCLAQLFL - YGRKKRRQRRR | 4 |
| Biotin- a2N(1-17)-TAT | Biotin- MGSLFRSESMCLAQLFL - YGRKKRRQRRR | 4 |
| Fluor- a2N(1-17)-TAT | Fluor- MGSLFRSESMCLAQLFL - YGRKKRRQRRR | 4 |
| FITC- a2N(1-17)-TAT | FITC- MGSLFRSESMCLAQLFL - YGRKKRRQRRR | 4 |
| CTMR- a2N(1-17)-TAT | CTMR- MGSLFRSESMCLAQLFL - YGRKKRRQRRR | 4 |

a3-subunit V-ATPase, (Mus musculus: NP_001129563.1, NM_001136091.2)

| | | |
|---|---|---|
| a3N-01 or | MGSMFRSEEVALVQLLL | 5 |
| a3N(1-17) | | |
| Biotin- a3N(1-17) | Biotin- MGSMFRSEEVALVQLLL | 5 |
| a3N(1-17)-TAT | MGSMFRSEEVALVQLLL - YGRKKRRQRRR | 6 |
| Biotin- a3N(1-17)-TAT | Biotin- MGSMFRSEEVALVQLLL - YGRKKRRQRRR | 6 |
| Fluor- a3N(1-17)-TAT | Fluor- MGSMFRSEEVALVQLLL - YGRKKRRQRRR | 6 |
| FITC- a3N(1-17)-TAT | FITC- MGSMFRSEEVALVQLLL - YGRKKRRQRRR | 6 |
| CMTR- a3N(1-17)-TAT | CMTR- MGSMFRSEEVALVQLLL - YGRKKRRQRRR | 6 |

a4-subunit V-ATPase, (Mus musculus: NP_536715.3, NM_080467.3)

| | | |
|---|---|---|
| a4N-01 or | MASVFRSEEMCLSQVFL | 7 |
| a4N(1-17) | | |
| Biotin- a4N(1-17) | Biotin- MASVFRSEEMCLSQVFL | 7 |
| a4N(1-17)-TAT | MASVFRSEEMCLSQVFL - YGRKKRRQRRR | 8 |
| Biotin- a4N(1-17)-TAT | Biotin- MASVFRSEEMCLSQVFL - YGRKKRRQRRR | 8 |
| Fluor- a4N(1-17)-TAT | Fluor- MASVFRSEEMCLSQVFL - YGRKKRRQRRR | 8 |
| FITC- a4N(1-17)-TAT | FITC- MASVFRSEEMCLSQVFL - YGRKKRRQRRR | 8 |
| CMTR- a4N(1-17)-TAT | CMTR- MASVFRSEEMCLSQVFL - YGRKKRRQRRR | 8 |

Homo sapiens
a1-subunit V-ATPase, (Homo sapiens: NP_001123492.1, NM_001130020.1)

| | | |
|---|---|---|
| a1N-01 or | MGELFRSEEMTLAQLFL | 9 |
| a1N(1-17) | | |
| Biotin- a1N(1-17) | Biotin- MGELFRSEEMTLAQLFL | 9 |
| a1N(1-17)-TAT | MGELFRSEEMTLAQLFL - YGRKKRRQRRR | 10 |
| Biotin- a1N(1-17)-TAT | Biotin- MGELFRSEEMTLAQLFL - YGRKKRRQRRR | 10 |
| Fluor- a1N(1-17)-TAT | Fluor- MGELFRSEEMTLAQLFL - YGRKKRRQRRR | 10 |
| FITC- a1N(1-17)-TAT | FITC- MGELFRSEEMTLAQLFL - YGRKKRRQRRR | 10 |
| CMTR- a1N(1-17)-TAT | CMTR- MGELFRSEEMTLAQLFL - YGRKKRRQRRR | 10 |

a2-subunit V-ATPase, (Homo sapiens: NP_036595.2, NM_012463.3)

| | | |
|---|---|---|
| a2N-01 or | MGSLFRSETMCLAQLFL | 11 |
| a2N(1-17) | | |
| Biotin- a2N(1-17) | Biotin- MGSLFRSETMCLAQLFL | 11 |
| a2N(1-17)-TAT | MGSLFRSETMCLAQLFL - YGRKKRRQRRR | 12 |
| Biotin- a2N(1-17)-TAT | Biotin- MGSLFRSETMCLAQLFL - YGRKKRRQRRR | 12 |
| Fluor- a2N(1-17)-TAT | Fluor- MGSLFRSETMCLAQLFL - YGRKKRRQRRR | 12 |
| FITC- a2N(1-17)-TAT | FITC- MGSLFRSETMCLAQLFL - YGRKKRRQRRR | 12 |
| CTMR- a2N(1-17)-TAT | CMTR- MGSLFRSETMCLAQLFL - YGRKKRRQRRR | 12 |

a3-subunit V-ATPase, (Homo sapiens: NP_006010.2, NM_006019.3)

| | | |
|---|---|---|
| a3N-01 or | MGSMFRSEEVALVQLFL | 13 |
| a3N(1-17) | | |
| Biotin- a3N(1-17) | Biotin- MGSMFRSEEVALVQLFL | 13 |
| a3N(1-17)-TAT | MGSMFRSEEVALVQLFL - YGRKKRRQRRR | 14 |
| Biotin- a3N(1-17)-TAT | Biotin- MGSMFRSEEVALVQLFL - YGRKKRRQRRR | 14 |
| Fluor- a3N(1-17)-TAT | Fluor- MGSMFRSEEVALVQLFL - YGRKKRRQRRR | 14 |
| FITC- a3N(1-17)-TAT | FITC- MGSMFRSEEVALVQLFL - YGRKKRRQRRR | 14 |
| CMTR- a3N(1-17)-TAT | CMTR- MGSMFRSEEVALVQLFL - YGRKKRRQRRR | 14 |

a4-subunit V-ATPase, (Home sapiens: NP_065683.2, NM_020632.2)

| | | |
|---|---|---|
| a4N-01 or | MVSVFRSEEMCLSQLFL | 15 |
| a4N(1-17) | | |
| Biotin- a4N(1-17) | Biotin- MVSVFRSEEMCLSQLFL | 15 |
| a4N(1-17)-TAT | MVSVFRSEEMCLSQLFL - YGRKKRRQRRR | 16 |
| Biotin- a4N(1-17)-TAT | Biotin- MVSVFRSEEMCLSQLFL - YGRKKRRQRRR | 16 |
| Fluor- a4N(1-17)-TAT | Fluor- MVSVFRSEEMCLSQLFL - YGRKKRRQRRR | 16 |
| FITC- a4N(1-17)-TAT | FITC- MVSVFRSEEMCLSQLFL - YGRKKRRQRRR | 16 |
| CMTR- a4N(1-17)-TAT | CMTR- MVSVFRSEEMCLSQLFL - YGRKKRRQRRR | 16 |

TABLE 2
Mouse and Human V-ATPase a-subunit isoforms derived
Peptides aN-03 or aN (35-49)

| | | |
|---|---|---|
| | | SEQ |
| Name | Label & Sequence | ID NO: |

Mus musculus
a1-subunit V-ATPase, (Mus musculus: NP_058616.1, NM_016920.3;
NP_001229979.1, NM_001243050.1; NP_001229980.1, NM_001243051.1)

| | | |
|---|---|---|
| a1N-03 or | VQFRDLNPDVNVFQR | 17 |
| a1N(35-49) | | |
| Biotin- a1N(35-49) | Biotin- VQFRDLNPDVNVFQR | 17 |
| a1N(35-49)-TAT | VQFRDLNPDVNVFQR - YGRKKRRQRRR | 18 |
| Biotin- a1N(35-49)-TAT | Biotin- VQFRDLNPDVNVFQR - YGRKKRRQRRR | 18 |
| Fluor- a1N(35-49)-TAT | Fluor- VQFRDLNPDVNVFQR - YGRKKRRQRRR | 18 |
| FITC- a1N(35-49)-TAT | FITC- VQFRDLNPDVNVFQR - YGRKKRRQRRR | 18 |
| CMTR- a1N(35-49)-TAT | CMTR- VQFRDLNPDVNVFQR - YGRKKRRQRRR | 18 |

a2-subunit V-ATPase, (Mus musculus: NP_035726.2, NM_011596.4;
NP_035726.2, NM_011596.4)

| | | |
|---|---|---|
| a2N-03 or | VQFRDLNQNVSSFQR | 19 |
| a2N(35-49) | | |
| Biotin- a2N(35-49) | Biotin- VQFRDLNQNVSSFQR | 19 |
| a2N(35-49)-TAT | VQFRDLNQNVSSFQR - YGRKKRRQRRR | 20 |
| Biotin- a2N(35-49)-TAT | Biotin- VQFRDLNQNVSSFQR - YGRKKRRQRRR | 20 |
| Fluor- a2N(35-49)-TAT | Fluor- VQFRDLNQNVSSFQR - YGRKKRRQRRR | 20 |
| FITC- a2N(35-49)-TAT | FITC- VQFRDLNQNVSSFQR - YGRKKRRQRRR | 20 |
| CTMR- a2N(35-49)-TAT | CTMR- VQFRDLNQNVSSFQR - YGRKKRRQRRR | 20 |

a3-subunit V-ATPase, (Mus musculus: NP_001129563.1, NM_001136091.2)

| | | |
|---|---|---|
| a3N-03 or | VEFRDLNESVSAFQR | 21 |
| a3N(35-49) | | |
| Biotin- a3N(35-49) | Biotin - VEFRDLNESVSAFQR | 21 |
| a3N(35-49)-TAT | VEFRDLNESVSAFQR - YGRKKRRQRRR | 22 |
| Biotin- a3N(35-49)-TAT | Biotin- VEFRDLNESVSAFQR - YGRKKRRQRRR | 22 |
| Fluor- a3N(35-49)-TAT | Fluor- VEFRDLNESVSAFQR - YGRKKRRQRRR | 22 |
| FITC- a3N(35-49)-TAT | FITC- VEFRDLNESVSAFQR - YGRKKRRQRRR | 22 |
| CMTR- a3N(35-49)-TAT | CMTR- VEFRDLNESVSAFQR - YGRKKRRQRRR | 22 |

a4-subunit V-ATPase, (Mus musculus: NP_536715.3, NM_080467.3)

| | | |
|---|---|---|
| a4N-03 or | VQFKDLNANVNSFQR | 23 |
| a4N(35-49) | | |
| Biotin- a4N(35-49) | Biotin- VQFKDLNANVNSFQR | 23 |
| a4N(35-49)-TAT | VQFKDLNANVNSFQR - YGRKKRRQRRR | 24 |
| Biotin- a4N(35-49)-TAT | Biotin- VQFKDLNANVNSFQR - YGRKKRRQRRR | 24 |
| Fluor- a4N(35-49)-TAT | Fluor- VQFKDLNANVNSFQR - YGRKKRRQRRR | 24 |
| FITC- a4N(35-49)-TAT | FITC- VQFKDLNANVNSFQR - YGRKKRRQRRR | 24 |
| CMTR- a4N(35-49)-TAT | CMTR- VQFKDLNANVNSFQR - YGRKKRRQRRR | 24 |

Homo sapiens
a1-subunit V-ATPase, (Homo sapiens: NP_001123492.1, NM_001130020.1)

| | | |
|---|---|---|
| a1N-03 or | VQFRDLNPDVNVFQR | 25 |
| a1N(35-49) | | |
| Biotin- a1N(35-49) | Biotin- VQFRDLNPDVNVFQR | 25 |
| a1N(35-49)-TAT | VQFRDLNPDVNVFQR - YGRKKRRQRRR | 26 |
| Biotin- a1N(35-49)-TAT | Biotin- VQFRDLNPDVNVFQR - YGRKKRRQRRR | 26 |
| Fluor- a1N(35-49)-TAT | Fluor- VQFRDLNPDVNVFQR - YGRKKRRQRRR | 26 |
| FITC- a1N(35-49)-TAT | FITC- VQFRDLNPDVNVFQR - YGRKKRRQRRR | 26 |
| CMTR- a1N(35-49)-TAT | CMTR- VQFRDLNPDVNVFQR - YGRKKRRQRRR | 26 |

a2-subunit V-ATPase, (Homo sapiens: NP_036595.2, NM_012463.3)

| | | |
|---|---|---|
| a2N-03 or | VQFRDLNQNVSSFQR | 27 |
| a2N(35-49) | | |
| Biotin- a2N(35-49) | Biotin- VQFRDLNQNVSSFQR | 27 |
| a2N(35-49)-TAT | VQFRDLNQNVSSFQR - YGRKKRRQRRR | 28 |
| Biotin- a2N(35-49)-TAT | Biotin- VQFRDLNQNVSSFQR - YGRKKRRQRRR | 28 |
| Fluor- a2N(35-49)-TAT | Fluor- VQFRDLNQNVSSFQR - YGRKKRRQRRR | 28 |
| FITC- a2N(35-49)-TAT | FITC- VQFRDLNQNVSSFQR - YGRKKRRQRRR | 28 |
| CTMR- a2N(35-49)-TAT | CTMR- VQFRDLNQNVSSFQR - YGRKKRRQRRR | 28 |

a3-subunit V-ArPase, (Home sapiens: NP_006010.2, NM_006019.3)

| | | |
|---|---|---|
| a3N-03 or | VEFRDLNASVSAFQR | 29 |
| a3N(35-49) | | |
| Biotin- a3N(35-49) | Biotin- VEFRDLNASVSAFQR | 29 |
| a3N(35-49)-TAT | VEFRDLNASVSAFQR - YGRKKRRQRRR | 30 |
| Biotin- a3N(35-49)-TAT | Biotin- VEFRDLNASVSAFQR - YGRKKRRQRRR | 30 |
| Fluor- a3N(35-49)-TAT | Fluor- VEFRDLNASVSAFQR - YGRKKRRQRRR | 30 |
| FITC- a3N(35-49)-TAT | FITC- VEFRDLNASVSAFQR - YGRKKRRQRRR | 30 |
| CMTR- a3N(35-49)-TAT | CMTR- VEFRDLNASVSAFQR - YGRKKRRQRRR | 30 |

a4-subunit V-ArPase, (Home sapiens: NP_065683.2, NM_020632.2)

| | | |
|---|---|---|
| a4N-03 or | VQFKDLNMNVNSFQR | 31 |
| a4N(35-49) | | |
| Biotin- a4N(35-49) | Biotin- VQFKDLNMNVNSFQR | 31 |
| a4N(35-49)-TAT | VQFKDLNMNVNSFQR - YGRKKRRQRRR | 32 |
| Biotin- a4N(35-49)-TAT | Biotin- VQFKDLNMNVNSFQR- YGRKKRRQRRR | 32 |
| Fluor- a4N(35-49)-TAT | Fluor- VQFKDLNMNVNSFQR - YGRKKRRQRRR | 32 |
| FITC- a4N(35-49)-TAT | FITC- VQFKDLNMNVNSFQR - YGRKKRRQRRR | 32 |
| CMTR- a4N(35-49)-TAT | CMTR- VQFKDLNMNVNSFQR - YGRKKRRQRRR | 32 |

In particular, the a1-subunit isoform is highly expressed in neuronal tissue. In nerve terminals, the a 1-isoform is specifically targeted to synaptic vesicles in which V-ATPase is involved in uptake of various neurotransmitters and their release upon stimulation (Morel et al. (J Cell Sci 116, 4751-4762, 2003); Saw et al. (Mol Biol Cell 22, 3394-3409, 2011)). However, in neuronal cells a1-isoform could also be targeted to compartments of endocytic pathways. For example, in presenilin-1 (PS1) null blastocysts, neurons from mice hypomorphic for or conditionally depleted of PS1, the a1-isoform is involved in targeting of V-ATPase to lysosomes (Lee et al. (Cell 141, 1146-1158, 2010)). That study identified the cell biological mechanism of a1-isoform targeting to lysosomes, in which physical interaction of unglycosylated a1-isoform with PS1 is required for its N-glycosylation and efficient delivery from ER to lysosomes. In previous studies, the a2-isoform was found to be targeting V-ATPase to the early endosomes of megalin/cubilin-receptor mediated protein degradative pathway in kidney proximal tubules both in situ and in MTC cultured cells (Hurtado-Lorenzo et al. (Nat Cell Biol 8, 124-136, 2006); Recchi and Chavrier (Nat Cell Biol 8, 107-109, 2006); Marshansky (Biochem Soc Trans 35, 1092-1099, 2007)). In MTC cells, the a2-isoform is not targeted to the Golgi, while in cultured osteoclast cells (Toyomura et al. (J Biol Chem 278, 22023-22030, 2003)) and B16 cells (Sun-Wada and Wada (Histol Histopathol 25, 1611-1620, 2010)), both endogenous a2- and a1-isoforms are targeted to the Golgi complex. In addition, in neuroendocrine PC12 cells, the stable expression of fluorescence-tagged a2-isoform (a2-EmGFP) also revealed targeting of V-ATPase with this subunit to the Golgi apparatus (Saw et al. (Mol Biol Cell 22, 3394-3409, 2011)).

The a3-isoform was identified as osteoclast lysosomal specific and could be relocated to the plasma membrane during osteoclast differentiation (Toyomura et al. (J Biol Chem 278, 22023-22030, 2003); Sun-Wada and Wada (Histol Histopathol 25, 1611-1620, 2010); Sun-Wada et al. (J Cell Sci 122, 2504-2513, 2009)). Recent studies demonstrated that nascent phagosomes progressively acquire a3-subunit containing V-ATPase from lysosomes during bacterial infection (Sun-Wada et al. (J Cell Sci 122, 2504-2513, 2009)). However, in neuroendocrine PC12 cells, the stably expressed a3-EmGFP was targeted to early endosomes of the endocytic pathway, while this endogenous a3-isoform was specifically targeted to insulin containing secretory granules of the exocytic pathway in pancreatic β-cells (Marshansky and Futai (Curr Opin Cell Biol 20, 415-426, 2008)).

In contrast to tissue ubiquitous a1-, a2-, and a3-isoforms, the expression of a4-isoform is highly specific for kidney, cochlea, and epididymis. Moreover, in those tissues, the a4-isoform is specifically targeted to the apical plasma membrane of collecting duct intercalated and epididymal clear cells (Pietrement et al. (Biol Reprod 74, 185-194, 2006)). Depending on the cell type the endosomal and lysosomal V-ATPase is targeted by the a1-, a2-, and a3-isoforms, differential function and acidification in these compartments can result. However, the mechanisms of their specific targeting are largely unknown and only recently have started emerging (Lee et al. (Cell 141, 1146-1158, 2010)). Recent studies on knockdown of specific a-isoforms revealed functional compensation by a-subunit isoforms, which may also take place in function of endocytic pathway under physiological conditions (Saw et al. (Mol Biol Cell 22, 3394-3409, 2011)). The V-ATPase itself functions as a pH-sensing receptor, which via interaction between the a1-, a2-, a3-, and a4-isoforms with cytohesin-2 and Arf6, modulate vesicular trafficking within the endosomal/lysosomal pathway (Marshansky and Futai (Curr Opin Cell Biol 20, 415-426, 2008)). This provides a mechanism of functional compensation by a-isoforms in regulation of acidification capacity of endosomal compartments. Cross-talk between V-ATPase and cytohesin-2/Arf6 demonstrates that these proteins function as “molecular on/off switches” in disassembly/assembly of V-ATPase and thus regulation of its function and acidification capacity endosomal/lysosomal compartments (Marshansky and Futai (Curr Opin Cell Biol 20, 415-426, 2008); Merkulova et al. (PLoS Computational Biology (Under revision, 2013); Merkulova et al. (Experimental Biology Meeting Abstract, Boston, 2013)).

aN peptides, are useful, e.g., as therapeutic agents, to inhibit the enzymatic GDP/GTP-exchange activity of cytohesin-2 to treat cancer and diabetic nephropathy. The peptides can be synthesized using peptide synthesis methods known in the art, or can be recombinant (e.g., expressed in a cell or animal and isolated and/or purified therefrom using methods known in the art). In some embodiments, the peptide has an amino acid sequence that has at least 90%, 95%, 98%, or more identity to any one of SEQ ID NOs:1 to 32.

The aN peptides can be part of a fusion protein, i.e., can be linked to a non-V-ATPase sequence. The non-V-ATPase sequence can be, e.g., a cell penetrating peptide or a label, e.g., a fluorescent protein such as GFP or a variant thereof. The non-V-ATPase polypeptide can be fused to the N-terminus or C-terminus of the aN peptide.

The fusion protein can include a moiety which has a high affinity for a ligand. For example, the fusion protein can be a GST-aN fusion protein in which the aN sequences are fused to the C-terminus of the GST sequences. Such fusion proteins can facilitate the purification of recombinant aN peptides. Alternatively, the fusion protein can be an aN peptide containing a heterologous signal sequence at its N-terminus. In certain host cells (e.g., mammalian host cells), expression and/or secretion of aN can be increased through use of a heterologous signal sequence.

In some embodiments, the fusion protein includes a cell-penetrating peptide (CPP) sequence that facilitates delivery of the aN peptide to the intracellular space, e.g., HIV-derived TAT peptide, penetratins, transportans, or hCT derived cell-penetrating peptides, see, e.g., Caron et al., (2001) Mol Ther. 3(3):310-8; Langel, Cell-Penetrating Peptides: Processes and Applications (CRC Press, Boca Raton Fla. 2002); El-Andaloussi et al., (2005) Curr Pharm Des. 11(28):3597-611; and Deshayes et al., (2005) Cell Mol Life Sci. 62(16): 1839-49. Fusion proteins can include all or a part of a serum protein, e.g., an IgG constant region, or human serum albumin.

Expression vectors are commercially available that already encode a fusion moiety (e.g., a GST polypeptide). An aN peptide-encoding nucleic acid can be cloned into such an expression vector such that the fusion moiety is linked in-frame to the aN peptide.

In some embodiments, the peptide is coupled (i.e., physically linked) to a detectable substance (i.e., labeled). Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate (FITC), rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S, or ³H.

The aN peptides and fusion proteins can be incorporated into pharmaceutical compositions and administered to a subject in vivo. aN peptides and fusion proteins can be useful therapeutically for the treatment of the diseases described herein, e.g., cancer treatment and inhibition of metastatic invasion; and diabetic nephropathy.

The invention includes vectors, preferably expression vectors, containing a nucleic acid that encodes the peptides and fusion proteins described herein. As used herein, the term “vector” refers to a nucleic acid molecule capable of transporting another nucleic acid to which it has been linked and can include, e.g., a plasmid, cosmid, or viral vector. The vector can autonomously replicate or it can integrate into a host cell's DNA. Viral vectors include, e.g., replication-defective retroviruses, adenoviruses, and adeno-associated viruses.

A vector can include an aN nucleic acid in a form suitable for expression of the nucleic acid in a host cell. Preferably a recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those that direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce aN peptides encoded by nucleic acids as described herein.

The recombinant expression vectors of the invention can be designed for expression of aN peptides in prokaryotic or eukaryotic cells. For example, peptides of the invention can be expressed in E. coli, insect cells (e.g., using baculovirus expression vectors), yeast cells, or mammalian cells (e.g., CHO or COS cells). Suitable host cells are discussed further in Goeddel, (1990) Gene Expression Technology: Methods in Enzymology 185, Academic Press, San Diego, Calif. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, Gene 67:31-40, 1988), μMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

One can maximize recombinant protein expression in E. coli by expressing the protein in host bacteria with an impaired capacity to proteolytically cleave the recombinant protein (Gottesman (1990) Gene Expression Technology: Methods in Enzymology 185:119-128, Academic Press, San Diego, Calif.). Another strategy is to alter the nucleic acid sequence of the nucleic acid to be inserted into an expression vector so that the individual codons for each amino acid are those preferentially utilized in E. coli (Wada et al., Nucleic Acids Res 20:2111-2118, 1992). Such alteration of nucleic acid sequences of the invention can be carried out by standard DNA synthesis techniques.

Modified versions of peptides disclosed herein are referred to as “peptide derivatives,” and they can also be used in the new methods. For example, peptide derivatives of a peptide can be used instead of that peptide in therapeutic methods described herein. Peptides disclosed herein can be modified according to the methods known in the art for producing peptidomimetics. See, e.g., Kazmierski, W. M., ed., Peptidomimetics Protocols, Human Press (Totowa N.J. 1998); Goodman et al., eds., Houben-Weyl Methods of Organic Chemistry: Synthesis of Peptides and Peptidomimetics, Thiele Verlag (New York 2003); and Mayo et al., J. Biol. Chem. 278:45746, 2003. In some cases, these modified peptidomimetic versions of the peptides and fragments disclosed herein exhibit enhanced stability in vivo, relative to the non-peptidomimetic peptides.

Methods for creating a peptidomimetic include substituting one or more, e.g., all, of the amino acids in a peptide sequence with D-amino acid enantiomers. Such sequences are referred to herein as “retro” sequences. In another method, the N-terminal to C-terminal order of the amino acid residues is reversed, such that the order of amino acid residues from the N-terminus to the C-terminus of the original peptide becomes the order of amino acid residues from the C-terminus to the N-terminus in the modified peptidomimetic. Such sequences can be referred to as “inverso” sequences.

Peptidomimetics can be both the retro and inverso versions, i.e., the “retro-inverso” version of a peptide disclosed herein. The new peptidomimetics can be composed of D-amino acids arranged so that the order of amino acid residues from the N-terminus to the C-terminus in the peptidomimetic corresponds to the order of amino acid residues from the C-terminus to the N-terminus in the original peptide.

Other methods for making a peptidomimetics include replacing one or more amino acid residues in a peptide with a chemically distinct but recognized functional analog of the amino acid, i.e., an artificial amino acid analog. Artificial amino acid analogs include β-amino acids, β-substituted β-amino acids (“β³-amino acids”), phosphorous analogs of amino acids, such as α-amino phosphonic acids and α-amino phosphinic acids, and amino acids having non-peptide linkages. Artificial amino acids can be used to create peptidomimetics, such as peptoid oligomers (e.g., peptoid amide or ester analogues), β-peptides, cyclic peptides, oligourea or oligocarbamate peptides; or heterocyclic ring molecules.

Nucleic acids disclosed herein also include both RNA and DNA, including genomic DNA and synthetic (e.g., chemically synthesized) DNA. Nucleic acids can be double-stranded or single-stranded. Nucleic acids can be synthesized using oligonucleotide analogs or derivatives (e.g., inosine or phosphorothioate nucleotides). Such oligonucleotides can be used, for example, to prepare nucleic acids with increased resistance to nucleases.

The term “purified” refers to an aN nucleic acid or an aN peptide that is substantially free of cellular or viral material with which it is naturally associated, or culture medium (when produced by recombinant DNA techniques), or chemical precursors or other chemicals (when chemically synthesized). Moreover, an isolated nucleic acid fragment is a nucleic acid fragment that is not naturally occurring as a fragment and would not be found in the natural state.

In some embodiments, the invention includes nucleic acid sequences that are substantially identical to an aN nucleic acid. A nucleic acid sequence that is “substantially identical” to an aN nucleic acid is at least 90% identical (e.g., at least about 95%, 96%, 97%, 98%, 99%, or identical) to an aN nucleic acid sequence.

To determine the percent identity of two amino acid or nucleic acid sequences, the sequences are aligned for optimal comparison purposes (i.e., gaps can be introduced as required in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=# of identical positions/total # of overlapping positions×100). The two sequences may be of the same length.

The percent identity or homology between two sequences can be determined using a mathematical algorithm. A non-limiting example of a mathematical algorithm utilized for the comparison of two sequences is the algorithm of Karlin and Altschul, Proc. Natl. Acad. Sci. USA 87:2264-2268, 1990, modified as in Karlin and Altschul, Proc. Natl. Acad. Sci. USA 90:5873-5877, 1993. Such an algorithm is incorporated into the NBLAST and XBLAST programs of Altschul et al., J Mol Biol 215:403-410, 1990. BLAST nucleotide searches can be performed with the NBLAST program, score=100, wordlength=12 to obtain nucleotide sequences homologous to aN nucleic acid molecules of the invention. BLAST protein searches can be performed with the XBLAST program, score=50, wordlength=3 to obtain amino acid sequences homologous to aN peptide molecules of the invention. To obtain gapped alignments for comparison purposes, Gapped BLAST can be utilized as described in Altschul et al., Nucleic Acids Res. 25:3389-3402, 1997. When utilizing BLAST and Gapped BLAST programs, the default parameters of the respective programs (e.g., XBLAST and NBLAST) can be used. See online at ncbi.nlm.nih.gov.

In other embodiments, the invention includes variants, homologs, and/or fragments of certain aN nucleic acids, e.g., variants, homologs, and/or fragments of an aN nucleic acid sequence. The terms “variant” or “homolog” in relation to aN nucleic acids include any substitution, variation, modification, replacement, deletion, or addition of one (or more) nucleotides from or to the sequence of an aN nucleic acid. The resultant nucleotide sequence may encode an aN peptide that has at least 50% of a biological activity (e.g., inhibition of cytohesin-2) of a referenced aN peptide (e.g., SEQ ID NOs: 1-32). In particular, the term “homolog” covers homology with respect to structure and/or function as long as the resultant nucleotide sequence encodes or is capable of encoding an aN peptide that has at least 50% of the biological activity of aN encoded by a sequence shown herein as SEQ ID NO: 1. With respect to sequence homology, there is at least 75% (e.g., 85%, 90%, 92%, 95%, 96%, 97%, 98%, 99%, or 100%) homology to the sequence shown as SEQ ID NOs: 1-32. The term “homology” as used herein can be equated with the term “identity.”

“Substantial homology” or “substantially homologous,” where homology indicates sequence identity, means at least 90% identical (e.g., at least about 92%, 95%, 96%, 97%, 98%, or 99%) sequence identity, as judged by direct sequence alignment and comparison. “Substantial homology” when assessed by the BLAST algorithm equates to sequences which match with an EXPECT value of at least about 7, e.g., at least about 9, 10, or more. The default threshold for EXPECT in BLAST searching is usually 10.

The invention also includes nucleic acids that hybridize, e.g., under stringent hybridization conditions (as defined herein) to all or a portion of the aN nucleotide sequences. The hybridizing portion of the hybridizing nucleic acids is typically at least 15 (e.g., 20, 25, or 30) nucleotides in length. The hybridizing portion of the hybridizing nucleic acid is at least about 80%, e.g., at least about 90%, 92%, 95%, 96%, 97%, 98%, 99%, or 100%, identical to the sequence of a portion or all of a nucleic acid encoding an aN peptide, or to its complement. Hybridizing nucleic acids of the type described herein can be used as a cloning probe, a primer (e.g., a PCR primer), or a diagnostic probe. Nucleic acids that hybridize to aN nucleotide sequences are considered “antisense oligonucleotides.”

High stringency conditions are hybridizing at 68° C. in 5×SSC/5×Denhardt's solution/1.0% SDS, or in 0.5 M NaHPO₄ (pH 7.2)/1 mM EDTA/7% SDS, or in 50% formamide/0.25 M NaHPO₄ (pH 7.2)/0.25 M NaCl/1 mM EDTA/7% SDS; and washing in 0.2×SSC/0.1% SDS at room temperature or at 42° C., or in 0.1×SSC/0.1% SDS at 68° C., or in 40 mM NaHPO₄ (pH 7.2)/1 mM EDTA/5% SDS at 50° C., or in 40 mM NaHPO₄ (pH 7.2) 1 mM EDTA/I % SDS at 50° C. Stringent conditions include washing in 3×SSC at 42° C. The parameters of salt concentration and temperature can be varied to achieve the optimal level of identity between the probe and the target nucleic acid. Additional guidance regarding such conditions is available in the art, for example, by Sambrook et al., 1989, Molecular Cloning, A Laboratory Manual, Cold Spring Harbor Press, N.Y.; and Ausubel et al. (eds.), 1995, Current Protocols in Molecular Biology, (John Wiley & Sons, N.Y.) at Unit 2.10.

Also included in the invention are genetic constructs (e.g., vectors and plasmids) that include an aN nucleic acid described herein, operably linked to a transcription and/or translation sequence to enable expression, e.g., expression vectors. A selected nucleic acid, e.g., a DNA molecule encoding an aN peptide, is “operably linked” to another nucleic acid molecule, e.g., a promoter, when it is positioned either adjacent to the other molecule or in the same or other location such that the other molecule can control transcription and/or translation of the selected nucleic acid.

Also included in the invention are various engineered cells, e.g., transformed host cells, which contain an aN nucleic acid described herein. A transformed cell is a cell into which (or into an ancestor of which) has been introduced, by means of recombinant DNA techniques, a nucleic acid encoding an aN peptide. Both prokaryotic and eukaryotic cells are included. Mammalian cells transformed with an aN nucleic acid can include host cells for an attaching enteric organism, e.g., intestinal cells, HeLa cells, and mouse embryonic fibroblasts. Prokaryotic cells can include bacteria, e.g., Escherichia coli. An engineered cell exemplary of the type included in the invention is an E. coli strain that expresses aN.

Certain aN peptides are also included within the present invention. Examples of such aN peptides are aN peptides and fragments, such as the ones shown as SEQ ID NOs: 1-32. The terms “protein” and “peptide” both refer to any chain of amino acids, regardless of length or post-translational modification (e.g., glycosylation or phosphorylation). Thus, the terms “aN protein,” and “aN peptide,” include full-length naturally occurring isolated proteins, as well as recombinantly or synthetically produced peptides that correspond to the full-length naturally occurring proteins, or to a fragment of the full-length naturally occurring or synthetic peptide.

As discussed above, the term “aN peptide” includes biologically active fragments of naturally occurring or synthetic aN peptides. Fragments of a protein can be produced by any of a variety of methods known to those skilled in the art, e.g., recombinantly, by proteolytic digestion, and/or by chemical synthesis. Internal or terminal fragments of a peptide can be generated by removing one or more nucleotides from one end (for a terminal fragment) or both ends (for an internal fragment) of a nucleic acid that encodes the peptide. Expression of such mutagenized DNA can produce peptide fragments. Digestion with “end-nibbling” endonucleases can thus generate DNAs that encode an array of fragments. DNAs that encode fragments of a protein can also be generated, e.g., by random shearing, restriction digestion, chemical synthesis of oligonucleotides, amplification of DNA using the polymerase chain reaction, or a combination of the above-discussed methods. Fragments can also be chemically synthesized using techniques known in the art, e.g., conventional Merrifield solid phase FMOC or t-Boc chemistry. For example, peptides of the present invention can be arbitrarily divided into fragments of desired length with no overlap of the fragments, or divided into overlapping fragments of a desired length.

A purified or isolated compound is a composition that is at least 75% by weight the compound of interest, e.g., an aN peptide. In general, the preparation is at least 80% (e.g., at least 90%, 95%, 96%, 97%, 98%, 99%, or 100%) by weight the compound of interest. Purity can be measured by any appropriate standard method, e.g., column chromatography, polyacrylamide gel electrophoresis, or HPLC analysis.

In certain embodiments, aN peptides include sequences substantially identical to all or portions of a naturally occurring aN peptide. Peptides “substantially identical” to the aN peptide sequences described herein have an amino acid sequence that has at least 90% (e.g., at least 92%, 95%, 96%, 97%, 98%, 99%, or 100%) identity to an amino acid sequence of an aN peptide represented by any one of SEQ ID NOs: 1-32.

In the case of peptide sequences that are less than 100% identical to a reference sequence, the non-identical positions are preferably, but not necessarily, conservative substitutions for the reference sequence. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Where a particular peptide is said to have a specific percent identity to a reference peptide of a defined length, the percent identity is relative to the reference peptide. Thus, a peptide that is 50% identical to a reference peptide that is 10 amino acids long can be a 5 amino acid peptide that is completely identical to a 5 amino acid long portion of the reference peptide. It also might be a 10 amino acid long peptide that is 50% identical to the reference peptide over its entire length.

aN peptides of the invention include, but are not limited to, recombinant peptides and natural peptides. Also included are nucleic acid sequences that encode forms of AN peptides in which naturally occurring amino acid sequences are altered or deleted. Certain nucleic acids of the present invention may encode peptides that are soluble under normal physiological conditions.

Also within the invention are nucleic acids encoding fusion proteins in which a portion of an aN peptide is fused to an unrelated peptide (e.g., one or more non-VATPase sequences, e.g., a TAT sequence) to create a fusion protein. For example, the peptide can be fused to a hexa-histidine tag or a FLAG tag to facilitate purification of bacterially expressed peptides or to a hemagglutinin tag or a FLAG tag to facilitate purification of peptides expressed in eukaryotic cells. The invention also includes, for example, isolated peptides (and the nucleic acids that encode these peptides) that include a first portion and a second portion, the first portion includes, e.g., an aN peptide, and the second portion can be, for example, a peptide that facilitates entry into a cell. In one aspect, aN can be fused to TAT. TAT consists of one or more short sequences of positively charged lysines or arginines exposed on the protein surface. TAT contains a protein transduction domain, and is therefore known as a cell penetrating peptide. This domain allows TAT to enter cells by crossing the cell membrane. The amino acid sequence of the protein transduction domain is YGRKKRRQRRR (SEQ ID NO:33). The nuclear localization signal found within the domain, GRKKR (SEQ ID NO:34), mediates further translocation of TAT into the cell nucleus.

Since aN peptides activate the macropinocytosis pathway, they could be used to deliver a variety of compounds into a cell, including a nucleic acid, amino acid, peptide, polypeptide, antibody, small molecule, toxin, nanoparticle, or any combination thereof. The aN peptide can be linked, e.g., covalently bonded, to the compound. Alternatively, the compound could be administered independently from the aN peptide, where the compound is unlinked, i.e., unattached to the peptide.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500, about 500 to about 1000, about 300 to about 1000 Da, or about 100 to about 250 Da).

In one aspect, anti-cytohesin small molecules including SecinH3, Secin16, Secin69, Secin107, and Secin132 are chemically attached to the aN peptide. This modification allows their more specific and potent targeting of the Sec7 domain and cytohesins, which could be useful to treat cancers, e.g., lung cancer, and may significantly potentiate action of both: i) the V-ATPase derived anti-cytohesin aN peptides; and ii) the anti-cytohesin small molecules (e.g., SecinH3, Secin16, Secin69, Secin107, and/or Secin132). Importantly, modifications of aN peptides with small molecules (e.g., SecinH3, Secin16, Secin69, Secin107, and/or Secin132) could be used not only for treatment of: i) cancer (cytohesin-dependent proliferation), but also, ii) to control proximal tubules proliferation in diabetes (DN); iii) insulin-receptor signaling in diabetes; and iv) cytohesin-dependent cell adhesion related diseases.

In all of the methods described herein, appropriate dosages of aN can readily be determined by those of ordinary skill in the art of medicine, e.g., by monitoring the patient for signs of disease amelioration or inhibition, and increasing or decreasing the dosage and/or frequency of treatment as desired. Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

For the compounds described herein, an effective amount, e.g., of a peptide (i.e., an effective dosage), ranges from about 0.001 to 30 mg/kg body weight, e.g., about 0.01 to 25 mg/kg body weight, e.g., about 0.1 to 20 mg/kg body weight. The peptide can be administered one time per day, twice per day, one time per week, twice per week, for between about 1 to 52 weeks per year, e.g., between 2 to 50 weeks, about 6 to 40 weeks, or for about 4, 5, or 6 weeks. The skilled artisan will appreciate that certain factors influence the dosage and timing required to effectively treat a patient, including but not limited to the type of patient to be treated, the severity of the disease or disorder, previous treatments, the general health and/or age of the patient, and other diseases present. Moreover, treatment of a patient with a therapeutically effective amount of a protein, polypeptide, antibody, nucleic acid, or other compound can include a single treatment or, preferably, can include a series of treatments.

### Methods of Treating Cancer

The invention described herein is based in part on the discovery that aN peptides and fusion proteins can be used to inhibit the enzymatic GDP/GTP-exchange activity of cytohesin-2 to treat epithelial cancer or carcinoma. Accordingly, in some embodiments of the methods described herein, methods of treating lung cancer, e.g., NSCLC, pancreatic cancer, squamous cell carcinomas of the head and neck, prostate cancer, breast cancer, colon cancer, kidney cancer, liver cancer, and brain cancer, are provided. The methods can involve selecting or diagnosing a subject having or at risk for developing cancer; and administering to the subject a therapeutically effective amount of a cytohesin-2 inhibitor, wherein the cytohesin-2 inhibitor comprises a peptide consisting of an amino acid sequence that has at least 90%, e.g., 95% or 98%, identity to the amino acid sequence of SEQ ID NO:9, 11, 13, 15, 25, 27, 29, or 31; or a fusion polypeptide comprising a first portion consisting of an amino acid sequence that has at least 90%, e.g., 95% or 98%, identity to the amino acid sequence of SEQ ID NO:9, 11, 13, 15, 25, 27, 29, or 31; and at least a second portion linked to the first portion, wherein the second portion comprises one or more non-VATPase sequences, e.g., a peptide consisting of an amino acid sequence that has at least 90%, e.g., 95% or 98%, identity to the amino acid sequence of SEQ ID NO: 10, 12, 14, 16, 26, 28, 30, or 32. The subject can be further monitored for treatment response.

In some embodiments of any of the methods described herein, the subject is suspected of having, is at risk of having, or has cancer, e.g., lung cancer, colon cancer, prostate cancer, breast cancer, kidney cancer, liver cancer, brain cancer, leukemia, lymphoma, multiple myeloma, pancreatic cancer, renal cancer, stomach cancer, bone cancer, hematological cancer, neural tissue cancer, melanoma, thyroid cancer, ovarian cancer, testicular cancer, cervical cancer, vaginal cancer, bladder cancer, carcinoma, sarcoma, metastatic disorders, and hematopoietic neoplastic disorders.

The term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, non-small cell carcinoma of the lung, renal-cell carcinoma, prostate cancer and/or testicular tumors, cancer of the small intestine, and cancer of the esophagus. Cancer that is “naturally arising” includes any cancer that is not experimentally induced by implantation of cancer cells into a subject, and includes, for example, spontaneously arising cancer, cancer caused by exposure of a subject to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures.

The term “sarcoma” is art-recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

A metastatic tumor can arise from a multitude of primary tumor types, including but not limited to those of lung, colon, prostate, breast, bone, brain, kidney, pancreas, and liver origin. Metastases develop, e.g., when tumor cells shed from a primary tumor adhere to vascular endothelium, penetrate into surrounding tissues, and grow to form independent tumors at sites separate from a primary tumor.

Cancers that may be treated using the methods and compositions of the present invention include, for example, cancers of the lung, stomach, colon, rectum, mouth/pharynx, esophagus, larynx, liver, pancreas, breast, cervix uteri, corpus uteri, ovary, prostate, testis, bladder, skin, bone, kidney, brain/central nervous system, head, neck and throat; Hodgkins disease, non-Hodgkins leukemia, sarcomas, choriocarcinoma, and lymphoma, among others.

A subject that is “suspected of having cancer” is one having one or more symptoms of the condition. Symptoms of cancer vary greatly and are well-known to those of skill in the art and include, without limitation, breast lumps, nipple changes, breast cysts, breast pain, weight loss, weakness, excessive fatigue, difficulty eating, loss of appetite, chronic cough, worsening breathlessness, coughing up blood, blood in the urine, blood in stool, nausea, vomiting, liver metastases, lung metastases, bone metastases, abdominal fullness, bloating, fluid in peritoneal cavity, vaginal bleeding, constipation, abdominal distension, perforation of colon, acute peritonitis (infection, fever, or pain), pain, vomiting blood, heavy sweating, fever, high blood pressure, anemia, diarrhea, jaundice, dizziness, chills, muscle spasms, colon metastases, lung metastases, bladder metastases, liver metastases, bone metastases, kidney metastases, pancreas metastases, difficulty swallowing, and the like. A subject that is “at risk of having cancer” is one that has a predisposition to develop cancer (i.e., a genetic predisposition develop cancer such as a mutation in a tumor suppressor gene, e.g., BRCA1, p53, RB, or APC) or has been exposed to conditions that can result in cancer. Thus, a subject that is “at risk of having cancer” can be one that has been exposed to mutagenic or carcinogenic levels of certain compounds (e.g., carcinogenic compounds in cigarette smoke such as acrolein, arsenic, benzene, benz {a}anthracene, benzo{a}pyrene, polonium-210 (Radon), urethane, or vinyl chloride). Moreover, the subject can be “at risk of having cancer” when the subject has been exposed to, e.g., large doses of ultraviolet light or X-irradiation, or exposed (e.g., infected) to a tumor-causing/associated virus such as papillomavirus, Epstein-Barr virus, hepatitis B virus, or human T-cell leukemia-lymphoma virus. Cancers are frequently treated with any of a variety of chemotherapeutic agents, which can be administered in conjunction with an aN peptide as disclosed herein.

Subjects considered at risk for developing cancer may benefit particularly from the invention, primarily because prophylactic treatment can begin before there is any evidence of the disorder. Subjects “at risk” include, e.g., subjects exposed to carcinogens, e.g., by consumption, e.g., by inhalation and/or ingestion, at levels that have been shown statistically to promote cancer in susceptible subjects. Also included are subjects at risk due to exposure to ultraviolet radiation, or their environment, occupation, and/or heredity, as well as those who show signs of a precancerous condition such as polyps. Similarly, subjects in very early stages of cancer or development of metastases (i.e., only one or a few aberrant cells are present in the subject's body or at a particular site in a subject's tissue) may benefit from such prophylactic treatment.

Skilled practitioners will appreciate that a subject can be diagnosed by a physician (or veterinarian, as appropriate for the subject being diagnosed) as suffering from or at risk for a condition described herein, e.g., cancer, by any method known in the art, e.g., by assessing a subject's medical history, performing diagnostic tests, and/or by employing imaging techniques.

Skilled practitioners will also appreciate that compositions comprising a cytohesin-2 inhibitor, e.g., an aN peptide, need not be administered to a subject by the same individual who diagnosed the subject (or prescribed the cytohesin-2 inhibitor for the subject). The cytohesin-2 inhibitor can be administered (and/or administration can be supervised), e.g., by the diagnosing and/or prescribing individual, and/or any other individual, including the subject her/himself (e.g., where the subject is capable of self-administration).

Common chemotherapeutic agents and/or treatments can be used in conjunction with the cytohesin-2 inhibitor to treat cancer. For example, cetuximab, panitumumab, gefitinib, erlotinib, lapatinib, campothecin, doxorubicin, cisplatin, carboplatin, procarbazine, mechlorethamine, cyclophosphamide, adriamycin, ifosfamide, melphalan, chlorambucil, bisulfan, nitrosurea, dactinomycin, daunorubicin, bleomycin, plicomycin, mitomycin, etoposide, verampil, podophyllotoxin, tamoxifen, taxol, transplatinum, 5-flurouracil, vincristin, vinblastin, methotrexate, and an analog of any of the aforementioned.

### Methods of Treating Diabetic Nephropathy

Diabetic nephropathy is a major long-term complication of diabetes mellitus and is the leading indication for dialysis and kidney transplantation in the United States (Marks and Raskin (1998) Med Clin North Am 82:877-907). The development of diabetic nephropathy is seen in 25 to 50% of type 1 and type 2 diabetic individuals. Contributing risk factors associated with the development of diabetic nephropathy (and other renal disorders) in subjects with type 1 or type 2 diabetes include hyperglycemia, hypertension, altered glomerular hemodynamics, and increased or aberrant expression of various growth factors, including transforming growth factor-beta (TGFβ), insulin-like growth factor (IGF)-I, vascular endothelial growth factor-a (VEGF-A), and connective tissue growth factor (CTGF). See, e.g., Flyvbjerg (2000) Diabetologia 43:1205-23; Brosius (2003) Exp Diab Res 4:225-233; Gilbert et al. (2003) Diabetes Care 26:2632-2636; and International Publication No. WO 00/13706.

Current treatment strategies directed at slowing the progression of diabetic nephropathy using various approaches, including optimized glycemic control (through modification of diet and/or insulin therapy) and hypertension control, have demonstrated varying degrees of success. For example, both angiotensin-converting enzyme (ACE) inhibitors and angiotensin receptor blockers (ARBs), administered to reduce hypertension, have been shown to delay progression or development of nephropathy and macroalbuminuria. Several clinical trials have established the benefits of ACE inhibitors and ARBs in patients with diabetes. However, although ACE inhibitors have been shown to delay renal decline in patients with type 1 diabetes, the renoprotective effect of these agents in patients with type 2 diabetes is less clear (Raij (2003) Am J. Hypertens 16:46S-49S). Further, while glycemic and blood pressure control therapies significantly decrease the morbidity and mortality associated with diabetic nephropathy by delaying progression of associated pathologies, such conventional therapies do not adequately halt the progression of the disease and thus fail to provide a complete therapeutic effect. In addition, administration of ACE inhibitors or ARBs, are not universally effective and only minimally delay, but do not remove, the need for kidney transplantation.

Other treatment strategies have focused on one or more growth factors as therapeutic targets. Therapies directed at inhibiting VEGF or TGFβ, either alone or in combination with ACE inhibitors or ARBs, have been examined. See, e.g., De Vriese et al. (2001) J. Am Soc Nephrol 12:993-1000; Flyvbjerg et al. (2002) Diabetes 51:3090-3094; Ziyadeh et al, (2000) Proc Natl Acad Sci 97:8015-8020; Chen et al. (2003) Biochem Biophys Res Commun 300:16-22; and Benigni et al. (2003) J. Am Soc Nephrol 14:1816-1824. Such therapeutic approaches, however, have not provided amelioration of all aspects of renal pathology (e.g., altered and impaired renal function and structure) associated with diabetic nephropathy. For example, inhibition of TGFβ as a therapeutic target for diabetic nephropathy was not effective at attenuating albuminuria in db/db mice, despite the beneficial effects such treatment had on glomerular matrix expansion (See, Ziyadeh et al, supra). In addition, while administration of anti-VEGF antibodies to diabetic db/db mice provided benefit to diabetes-associated increased permeability in the kidney, only minimal beneficial effects on mesangial expansion were observed. See, Flyvbjerg et al (2002), supra. A recent study has identified cytohesin-2 as an important downstream regulator of the VEGF receptor signaling (Mannell et al., Cardiovascular Res 93:111-119, 2012), which is opening an alternative possibility of targeting of this pathway with anti-cytohesin-2 inhibitors. Therefore, although such therapies offer promise, none have resulted in amelioration of pathological features associated with diabetic nephropathy. Thus, there is a need in the art for a complete therapy for treatment of diabetic nephropathy that ameliorates both early and late stages symptoms and pathologies associated with the development and progression of the disease.

In addition to the above deficiencies, current therapies for diabetic nephropathy have limited applicability/efficacy due to lack of specificity. In particular, VEGF- or TGFβ-targeted therapies may compromise the beneficial activities of these growth factors, such as angiogenesis, tumor suppression, and proper immune system development. For example, while TGFβ has been associated with development of fibrosis, it is also an important mediator of immune development and tumor suppression, suggesting that inhibition of TGFβ might have unwanted and potentially adverse secondary effects. Therefore, there is a need in the art for a more selective therapeutic approach for diabetic nephropathy.

There is an existing need for a therapeutic approach to treat diabetic nephropathy that is effective at various stages (e.g., early stage and late stage diabetic nephropathy) in the development and progression of the disease. In particular, there is a need for a complete treatment for diabetic nephropathy, one effective in treating both early stage features and late stage features of diabetic nephropathy such as, for example, hyperfiltration (early stage), increased glomerular permeability (early stage), increased glomerular filtration rate (early stage), microalbuminuria (early stage), macroalbuminuria (late stage), and decreased glomerular filtration rate (late stage). There is a need for a therapeutic approach that more completely addresses various and distinct processes associated with development and progression of diabetic nephropathy. In particular, there is a need for therapies that target both non-fibrotic (e.g., hyperfiltration) and fibrotic (e.g., mesangial matrix expansion) processes associated with diabetic nephropathy.

The present invention addresses these needs by identifying a role of cytohesin-2 in the development and progression of diabetic nephropathy, and by providing methods and agents that can be applied to the treatment and prevention of renal disease associated with diabetes, and most particularly, diabetic nephropathy.

Diabetes is a major cause of morbidity and mortality worldwide with approximately 40% of all individuals with diabetes developing diabetic nephropathy, requiring either kidney dialysis or transplantation. Diabetes is the leading cause of end stage renal disease, and therefore, any individual diagnosed with diabetes is at risk for the development of diabetic nephropathy.

Progression of diabetic nephropathy is characterized by a fairly predictable pattern of events. Generally, the time course of development of diabetic nephropathy is as follows. Glomerular hyperfiltration and renal hypertrophy occur in the first years after the onset of diabetes and are reflected by increased glomerular filtration rate (e.g., from a normal glomerular filtration rate of about 120 ml/min to about 150 ml/min in humans). During the first five years of diabetes, pathological changes, such as glomerular hypertrophy, thickening of the glomerular basement membrane, and glomerular mesangial volume expansion are observed. Glomerular filtration rate gradually returns to normal. After five to ten years of diabetes, subjects may begin to excrete small amounts of albumin in the urine (microalbuminuria). Microalbuminuria is an important predictor of progression to overt diabetic nephropathy (characterized, in part, by macroalbuminuria or overt proteinuria). The basement membrane thickening and glomerular volume expansion seen in early stages of the disease can accumulate in late stage diabetic nephropathy, leading to obliteration of the capillary lumen, and, eventually, to glomerulosclerosis. Once overt diabetic nephropathy is present, a steady decline in the glomerular filtration rate occurs, and approximately half of individuals reach end-stage renal disease in 7 to 10 years.

Clinically, the stages of development and progression of diabetic nephropathy in humans have been well described. Stage I diabetic nephropathy is associated with increased kidney (i.e., glormerular) filtration (i.e., hyperfiltration, resulting from increased blood flow through the kidneys and glomeruli), increased glomerular filtration rate, glomerular hypertrophy, and enlarged kidneys. Stage II diabetic nephropathy is a clinically silent phase associated with continued hyperfiltration and kidney hypertrophy. Thickening of the glomerular basement membrane and mesangial expansion occurs. Stage III diabetic nephropathy (also known as incipient diabetic nephropathy) is associated with microalbuminuria and micro proteinuria. Microalbuminuria is defined as 30 to 300 mg/day urinary albumin in a 24-hour collection, 20-200 μg/min. urinary albumin, or 30 to 300 μg/mg creatinine in a spot collection. The kidneys progressively lose the ability to filter waste and blood levels of creatinine and urea-nitrogen increase. Glomerular basement membrane thickening and mesangial expansion continue to occur with increasing severity. Stage IV diabetic nephropathy (also known as overt diabetic nephropathy) is associated with macroalbuminuria (i.e., clinical albuminuria) and creatinine and blood urea-nitrogen (BUN) levels in the blood continue to rise. Macroalbuminuria is defined as greater than 300 mg/day urinary albumin in a 24-hour collection, greater than 200 μg/min urinary albumin, or greater than 300 μg/mg creatinine spot collection. Once overt diabetic nephropathy occurs, glomerular filtration rate gradually falls over a period of several years. Stage V diabetic nephropathy occurs with end-stage renal disease and kidney failure.

### Kidney Proximal Tubule Megalin/Cubilin-Mediated Endosomal/Lysosomal Proteins Degradation Pathway

Reabsorption of filtered proteins is of central importance for function of kidney proximal tubule epithelial cells. Trafficking and degradation of these proteins via endosomal/lysosomal degradation pathway is important for normal protein homeostasis and for maintenance of physiological levels of hormones, vitamins and cytokines in blood. Many low molecular weight filtered plasma proteins include: i) albumin; ii) transferrin; iii) vitamin-binding proteins; iv) cytokines; and iv) hormones such as insulin, as well as drugs, such as gentamycin and amicacin, that are reabsorbed in PT cells through receptor-mediated endocytosis (Marshansky et al. (Electrophoresis 18, 2661-2676, 1997); Christensen and Birn (Nat Rev Mol Cell Biol 3, 256-266, 2002); Nielsen et al. (Proc Natl Acad Sci USA 104, 5407-5412, 2007)). This process involves three apically located multiligand-binding receptors, megalin, cubilin, and amnionless (Marshansky et al. (Electrophoresis 18, 2661-2676, 1997); Christensen and Birn (Nat Rev Mol Cell Biol 3, 256-266, 2002); Christensen et al. (Pflugers Arch 458, 1039-1048, 2009); Nielsen and Christensen (Pediatr Nephrol 25, 813-822, 2010)). Megalin is a transmembrane glycoprotein that belongs to the low-density lipoprotein receptor gene family and functions as a low-selectivity, high-capacity scavenger receptor. The cubilin molecule together with the protein amnionless forms the cubam receptor. However, cubilin also interacts with megalin and may function as a dual-receptor complex. While the function of megalin and cubilin receptors as well as their ligand specificity is well studied, the role of amnionless in function of endocytic pathway is just emerging (Christensen and Birn (Nat Rev Mol Cell Biol 3, 256-266, 2002); Christensen et al. (Pflugers Arch 458, 1039-1048, 2009); Nielsen and Christensen (Pediatr Nephrol 25, 813-822, 2010)). It is also noteworthy that the existence of the alternative endocytic pathways including transcytosis or macropinocytosis in kidney PT epithelial cells is currently unknown. Thus, in kidney PT cells in situ as well as in cultured MTC and OK cells, megalin/cubilin receptors and their ligands enter cells exclusively via the constitutively operated CDE endosomal/lysosomal pathway (Christensen and Birn (Nat Rev Mol Cell Biol 3, 256-266, 2002); Nielsen et al. (Proc Natl Acad Sci USA 104, 5407-5412, 2007); Gekle et al. (Am J Physiol 272, F668-677, 1997)). In this pathway, the ligands are delivered via a clathrin-coated vesicle (CCV), to early endosomes, late endosomes, and then to lysosomes for degradation, while megalin/cubilin-receptors return to the plasma membrane via recycling endosomes. Recently, the differential role of megalin and cubilin in protein reabsorption was examined using conditional Cre-LoxP knockout technology in mice (Amsellem et al. (J Am Soc Nephrol 21, 1859-1867, 2010)). This important study revealed a critical role of cubilin in albumin handling, while megalin plays an important auxiliary role in the internalization of cubilin/albumin complexes. Importantly, using this new genetic model (Amsellem et al. (J Am Soc Nephrol 21, 1859-1867, 2010); Weyer et al. (Nephrol Dial Transplant 26, 3446-3451, 2011)), the authors demonstrated that normally a small amount of filtrated albumin is uniquely reabsorbed via megalin/cubilin-mediated endosomal/lysosomal pathway and delivered to lysosomes for degradation. It is noteworthy, that while the notion of small glomerular filtration of albumin was recently challenged, it has received an intensive critique and assessment as artifactual observation (Christensen et al. (Pflugers Arch 458, 1039-1048, 2009); Nielsen and Christensen (Pediatr Nephrol 25, 813-822, 2010); Amsellem et al. (J Am Soc Nephrol 21, 1859-1867, 2010); Christensen et al. (Kidney Int 72, 1192-1194, 2007); Gekle (Kidney Int 71, 479-481, 2007); de Borst (Kidney Int 72, 1409; author reply 1409-1410, 2007); Remuzzi et al. (Kidney Int 72, 1410-1411; author reply 1411, 2007)). Thus, this study using a genetically modified mouse model resolved this recent controversy by demonstrating: i) limited amounts of albumin filtration; and ii) its exclusive handling by megalin/cublin-mediated endosomal/lysosomal pathway, supporting previously reported revised data using both multiphoton microscopy (Peti-Peterdi (Am J Physiol Renal Physiol 296, F1255-1257, 2009); Tanner (Am J Physiol Renal Physiol 296, F1258-1265, 2009)) and micropuncture (Remuzzi et al. (Kidney Int 72, 1410-1411; author reply 1411, 2007)) approaches. Finally, another recent study using this conditional megalin/cubilin-deficient mice has also resolved another controversy by showing that generation of urinary albumin fragments does not require PT's uptake and degradation of albumin via endosomal/lysosomal pathway (Weyer et al. (J Am Soc Nephrol 23, 591-596, 2012)). In conclusion, this new genetic mouse model represents powerful tool to study this pathway in health and disease. Thus, while this model has already been essential in order to solve two controversies in kidney physiology, it will undoubtedly be useful to study the function and regulation of megalin/cubilin-mediated endosomal/lysosomal pathway under diabetic conditions.

### Diabetes, Glucose Handling by Kidney Proximal Tubules and Diabetic Nephropathy

In healthy individuals, glucose homeostasis is controlled via the tight hormonal regulation of glucose consumption by the central nervous and peripheral tissues. The kidney also has a unique role in maintenance of glucose blood level via its glomerular filtration and proximal tubular reabsorption. Two glucose/sodium co-transporters SGLT1 and SGLT2 are involved in reabsorption of glucose on the apical pole of proximal tubule cells followed by its passive cytosolic diffusion and delivery of glucose across basolateral membrane of PT's via GLUT1 and GLUT2 transporter (Chao and Henry (Nat Rev Drug Discov 9, 551-559, 2010); Vallon (Am J Physiol Cell Physiol 300, C6-8, 2011)). This function makes renal PT cells especially sensitive to variations of blood glucose levels and adaptive to its reabsorptive handling. Under normoglycemic conditions (5.5 mM/100 mg/dl) about 180 g is daily filtered and reabsorbed by proximal tubules practically without loss of the glucose in urine. However, under hyperglycemic conditions (16.5 mM/300 mg/dl) during diabetes the filtered glucose saturated the transport capacity of SGLT1 and SGLT2 transporters giving rise to “glucose toxicity” and accompany detrimental renal effect in diabetes (Vallon (Am J Physiol Regul Integr Comp Physiol 300, R1009-1022, 2011)). Diabetes is one of the leading causes of a slow deterioration of the kidneys (nephropathy) leading to end-stage renal disease (ESRD) and finally to kidney failure. In the United States, approximately 35% of patients who develop chronic renal failure have diabetes. About half of these patients have insulin-dependent diabetes (IDDM, or Type 1 diabetes) and the other half non-insulin-dependent diabetes (NIDDM, or Type 2 diabetes).

The pathogenesis of late stages of diabetic complications leading to diabetic nephropathy and ESRD are relatively well studied and include glomerular defects linked with progressive deterioration of proximal tubular function and tubulointerstitial inflammation and fibrosis. One of the features of late stages diabetic nephropathy is diminished proteins reabsorption by proximal tubules via megalin/cubilin-mediated endosomal/lysosomal protein degradative pathway leading to proteinuria. Subjects with IDDM are more likely to develop proteinuria (mainly albuminuria) than those with NIDDM (Hosteter ((Ed. Bennet J. C.,), 599-602 (W.B. Saunders Company, Philadelphia,) 1996)). Microalbuminuria (albumin excretion>30 mg/d) can predict the development of diabetic nephropathy in both IDDM and NIDDM, but persistent albuminuria (>300 mg/d) is the hallmark of diabetic nephropathy and is used for clinical diagnosis. Importantly, albuminuria is not only a consequence of diabetes, but is also a causative factor in the progressive renal failure associated with late stages of diabetes (Parving and Osterby ((Ed. Brenner, B. M.), 1731-1773 (W.B. Saunders Company, Philadelphia) 2000)). In particular, elevated urinary albumin has been implicated in the induction of proinflammatory chemokines and growth factors, followed by infiltration of macrophages, initiation of inflammation and development of fibrosis in diabetic kidney (Vallon (Am J Physiol Regul Integr Comp Physiol 300, R1009-1022, 2011)).

On the other hand, the pathogenesis of important early events that link poorly controlled diabetes with repeated events of high glucose levels remains largely unexplored. In particular, the role of proximal tubule in the early stages of pathogenesis of diabetic nephropathy was underestimated and its importance only recently has been appreciated (Vallon (Am J Physiol Regul Integr Comp Physiol 300, R1009-1022, 2011)). Following cell biological events have been recognized as a crucial in early hyperglycemia during diabetes: i) enhancement of the glucose reabsorption by proximal tubule cells; ii) unique growth and early hyperplasia of the proximal tubules; and iii) primary hyper-reabsorption by the proximal tubules. It was established that the enhanced glucose reabsorption and tubular growth contribute to primary proximal tubular hyper-reabsorption, while primary proximal tubular hyper-reabsorption also contributes to glomerular hyperfiltration (Vallon (Am J Physiol Regul Integr Comp Physiol 300, R1009-1022, 2011)). On the other hand, recent in vivo studies demonstrated that acute hyperglycemia gives rise to rapid and significant increase in primary glomerular permeability to macromolecules, thus, could inversely promote proximal tubule hyper-reabsorption and increase in function of megalin/cubilin endosomal/lysosomal protein degradative pathway. Described herein are the mechanism of the phenomenon and the emerging crucial roles of V-ATPase, cytohesin-2/Arf6 and aldolase in regulating the endocytic pathway and protein reabsorption during the early stages of diabetes.

### Role of V-ATPase, Cytohesin-2/Arf6 and Aldolase in Regulation of Megalin/Cubilin-Mediated Endosomal/Lysosomal Protein Degradation Pathway: Implications for Diabetic Nephropathy

In proximal tubule cells, acidification of endocytic compartments becomes increasingly acidic from early endosomes to late endosomes and finally to lysosomes (Marshansky et al. (Curr Opin Nephrol Hypertens 11, 527-537, 2002)). Intravesicular acidification of these endocytic compartments is also driven by proton pumping V-ATPase and could be inhibited by bafilomycin. It is functioning in conjunction with a parallel chloride conductance facilitated by CLC-5 and CLC-4 electrogenic Cl⁻/H⁺-exchangers as previously reviewed (Marshansky et al. (Curr Opin Nephrol Hypertens 11, 527-537, 2002); Marshansky and Futai (Curr Opin Cell Biol 20, 415-426, 2008)). In Dent disease and Fanconi syndrome, reduction of chloride-driven electrical shunt inhibits endosomal V-ATPase-dependent acidification giving rise to diminished function of megalin/cubilin-mediated endocytic pathway and urinary wasting of albumin (Marshansky et al. (Curr Opin Nephrol Hypertens 11, 527-537, 2002)). However, the biochemical mechanism which coupled deficient endosomal acidification with impaired endocytic trafficking remained unclear. A crucial link between V-ATPase-driven endosomal acidification and Arf GTP-binding proteins in regulation of the megalin/cubilin-mediated endosomal/lysosomal protein degradation pathway has been uncovered (Hurtado-Lorenzo et al. (Nat Cell Biol 8, 124-136, 2006); Recchi and Chavrier (Nat Cell Biol 8, 107-109, 2006)). This involves a direct and acidification-dependent interaction of V-ATPase with cytosolic Arf's and their regulatory proteins (Hurtado-Lorenzo et al. (Nat Cell Biol 8, 124-136, 2006)). The a2-subunit isoform of V-ATPase is targeted to early endosomes in the PT and directly interacts with the cytohesin-2 (CTH2), while the Arf6 is binding to the c-subunit of the V-ATPase. Thus, the a2-isoform containing endosomal V-ATPase was discovered as a novel pH-sensor, which interacts with cytohesin-2/Arf6 regulates endosomal protein trafficking (Hurtado-Lorenzo et al. (Nat Cell Biol 8, 124-136, 2006); Recchi and Chavrier (Nat Cell Biol 8, 107-109, 2006); Marshansky (Biochem Soc Trans 35, 1092-1099, 2007)). It is important to underline that the interaction of cytohesin-2 with the “endosomal” V-ATPase a2-subunit extended to the other a-isoforms (a1-, a3-, and a4-) of the V-ATPase (Merkulova et al. (Am J Physiol Cell Physiol 300, C1442-1455, 2011)). This new finding indicates that regulatory interaction of V-ATPases with cytohesin-2 and signaling between these proteins is a general cell biological phenomenon. Cross-talk between V-ATPase and cytohesin-2/Arf6 may also exist on lysosomes, where it can regulate function of V-ATPase. It is noteworthy that regulation of V-ATPase activity by assembly of V_{O}V₁ sectors onto the lysosomal membrane was previously demonstrated in mammalian dendritic cells (Trombetta et al. (Science 299, 1400-1403, 2003)). This mechanism is critical for lysosomal acidification, activation of proteases, protein degradation, and antigen presentation during cell maturation. On the other hand, this mechanism of regulating the V-ATPase function in lysosomes was also observed in cells in which EGFR/ErbB-pathway and mTORC1 pathways were activated by signaling with EGF-ligands (Xu et al, J Biol Chem 287:26409-26422, 2012; Zoncu et al., Science 334:678-683, 2011; Bar-Peled et al., Cell 150:1196-1208, 2012; Bar-Peled et al., Cell 151:1390, 2012). This mechanism is critical for lysosomal sensing of nutrients, cell proliferation, and/or apoptosis and autophagy, and thus for the development of large variety of human diseases.

Two structural elements have been identified to be involved in specific and high affinity association of the V-ATPase a2-subunit isoform with cytohesin-2: i) an N-terminal binding motif formed by the first seventeen amino acids of the a2N called a2N-01 or a2N(1-17) peptide and ii) an interaction pocket formed by the catalytic Sec7 and regulatory PB-domains of cytohesin-2 (Merkulova et al. (Biochim Biophys Acta 1797, 1398-1409, 2010)). Biacore analysis revealed a very strong binding affinity between this a2N(1-17) peptide and the Sec7-domain of cytohesin-2, with dissociation constant K_{D}=3.44×10⁻⁷ M, similar to the binding affinity K=3.13×10⁻⁷ M between wild-type a2N(wt) and the full length cytohesin-2 (wt) protein. These interactions appear crucial for signaling and regulation of cytohesin-2 enzymatic Arf-GEF activity by V-ATPase. Indeed, a part of V-ATPase a2N(1-17) potently modulates the enzymatic GDP/GTP-exchange activity of cytohesin-2 via direct binding with its Sec7 domain (Hosokawa et al. (J Biol Chem 288:5896-5913, 2013)). In live PT cells, this V-ATPase-derived, cell-penetrating, and biologically active a2N(1-17) peptide strongly activates the macropinocytosis recycling pathway, while inhibiting the endosomal/lysosomal protein degradation pathway. Thus, a novel function of V-ATPase as a signaling receptor that modulates activity of the Arf-GEF cytohesin-2 and cognate Arf GTP-binding proteins has been uncovered. Signaling between the V-ATPase and Arfs may regulate crosstalk between distinct endocytic receptor-mediated and macropinocytosis pathways (Hosokawa et al. (Int J Cancer 2013, manuscript in preparation); Hosokawa et al. (Experimental Biology Meeting Abstract, Boston, 2013; Hosokawa et al. (“50th Annual Meeting of American Society for Cell Biology” (Philadelphia) Abstract of the Poster Presentation, 2010); Hosokawa et al. (“Gordon Research Conference: Bioenergetics” (Proctor Academy, Andover, N.H.) Abstract of the Poster Presentation and Invited Talk, 2011)).

Using a combination of protein-protein interaction techniques, cytohesin-2 was found to specifically interact with aldolase in MTC cells (Merkulova et al. (Am J Physiol Cell Physiol 300, C1442-1455, 2011)). The direct interaction of aldolase with cytohesin-2 via its PH-domain was further studied in pull-down and Biacore experiments. This approach revealed a two-step interaction between these two proteins with K_{D1}=1.1×10⁻⁴M and K_{D2}=2.7×10⁻⁶M, clearly indicating a potential regulatory mechanism of this interaction. Moreover, using an iodixanol gradient-based cell fractionation approach, aldolase, which is generally considered to be a predominantly cytosolic protein, was found to be associated with early endosomes containing the megalin/albumin-FITC receptor/ligand complexes, where it could directly interact with cytohesin-2 and V-ATPase (Merkulova et al. (Am J Physiol Cell Physiol 300, C1442-1455, 2011)). Thus, there exists a specific interaction between cytohesin-2 and aldolase in the V-ATPase/Arf6/cytohesin-2/aldolase complex on early endosomes, which regulates the activities of its components and function of the protein degradative pathway under physiologic and pathologic conditions (Marshansky et al. (Curr Opin Nephrol Hypertens 11, 527-537, 2002); Marshansky and Futai (Curr Opin Cell Biol 20, 415-426, 2008); Marshansky et al. (Electrophoresis 18, 2661-2676, 1997)).

The direct interaction between aldolase and cytohesin-2 are important in: i) gelsolin gene expression; ii) actin cytoskeletal rearrangement; and iii) redistribution of endosomal vesicles (Merkulova et al. (Am J Physiol Cell Physiol 300, C1442-1455, 2011)), demonstrating an important role of aldolase as a “glucose sensor” during early stages of diabetes in modulation of cytohesin-2/Arf6 signaling in gene expression, V-ATPase function, actin cytoskeleton rearrangement, and thus, regulation of proximal tubule endocytic pathway. Indeed, using non-obese diabetic (NOD) mice as a model of early stages of human Type 1 diabetes, an increase of expression and apical redistribution of both V-ATPase and megalin at early stages (2 weeks) of diabetes in NOD mice was observed. In addition, expression of cytohesin-2 in kidney proximal tubules of these diabetic mice was also significantly up-regulated. These results were further confirmed by quantitative RT-PCR and Western-blot analysis (Merkulova and Marshansky (“Gordon Conference on Molecular and Cellular Bioenergetics: From Crystal Structure to Human Disease” (Proctor Academy, Andover, N.H.) Abstract of the Poster Presentation and Invited Talk, 2009)). Thus, these data demonstrate that the protein degradation pathway in PT, represented by these critical components (cytohesin-2 and V-ATPase), is upregulated by high levels of glucose, which would affect its function during the early stages of diabetes.

Recent studies have provided new insights into regulation of megalin/cubilin-mediated endosomal/lysosomal protein degradation pathway under physiological and pathological conditions such as early stages of renal diabetic complication. A novel role of endosomal V-ATPase as pH-sensing receptor, which is involved in acidification-dependent transmembrane signaling and regulation of activity of cytohesin-2/Arf6 GTP-binding proteins was uncovered. Aldolase has also been identified as a novel modulator of V-ATPase/Arf6/cytohesin-2 complex, which may be involved in glucose-dependent regulation of gene expression, modulation of gelsolin/actin cytoskeleton, and function of V-ATPase-driven acidic endocytic compartments. The peptides described herein target the V-ATPase/Arf6/cytohesin-2/aldolase complex to normalize the function of megalin/cubilin-mediated endosomal/lysosomal protein degradation pathway during “glucose toxicity” in diabetes to treat or reduce a risk of developing DN.

A recent GWAS study on DN has identified ErbB4, a member of the EGFR/ErbB-family, as being strongly associated with development of DN caused by type 1 diabetes in humans (Sandholm et al. (PLoS Genet 8, e1002921, 2012); Boger and Sedor (PLoS Genet 8, e1002989, 2012)). ErbB4 has recently emerged as a crucial modulator of kidney PT proliferation and polarization during nephrogenesis (Veikkolainen et al. (J Am Soc Nephrol 23, 112-122, 2012); Zeng et al. (Mol Biol Cell 18, 4446-4456, 2007)). Recently cytohesin-2 has been identified as a critical cytoplasmic EGFR/ErbB receptor activator (FIG. 1) (Bill et al. (Cell 143, 201-211, 2010); Bill et al. (PLoS ONE 7, e41179, 2012)). Thus, the EGFR/ErbB/cytohesin-2 pathway could be involved in control of NSCLC and PT cell proliferation both in cancer and diabetes. It has been demonstrated that proliferation of the gefitinib-resistant NSCLC A549 and H460 cells is efficiently reduced by the small molecule SecinH3, a known inhibitor of cytohesin-2 (Bill et al. (PLoS ONE 7, e41179, 2012)). Moreover, treating mice bearing H460 as well as PC-9 xenografts with SecinH3 also showed an anti-proliferative and pro-apoptotic action of SecinH3 in vivo (Bill et al. (Cell 143, 201-211, 2010); Bill et al. (PLoS ONE 7, e41179, 2012)). Thus, cytohesin-2 is considered as a novel drug target for treatment of NSCLC, including TKI-resistant cancers (Cohen (J Biol Chem 237, 1555-1562, 1962); Cohen et al. (J Biol Chem 255, 4834-4842, 1980); Normanno et al. (Gene 366, 2-16, 2006); Zhang et al. (J Clin Invest 117, 2051-2058, 2007); Sharma and Settleman (Exp Cell Res 315, 557-571, 2009)), which also could be useful for treating or reducing a risk of developing DN (Sandholm et al. (PLoS Genet 8, e1002921, 2012); Boger and Sedor (PLoS Genet 8, e1002989, 2012)).

### EXAMPLES

The invention is further described in the following examples, which do not limit the scope of the invention described in the claims.

### Example 1

### The N-Terminus of a-Subunit Isoforms is Involved in Signaling Between V-ATPase and Cytohesin-2

Reagents and Antibodies—

If not otherwise specified, all reagents were purchased from Sigma-Aldrich. All buffers, NU-PAGE gels and lipofectamine 2000 transfection reagent were from Invitrogen. Western Lightning chemiluminescence reagent plus was obtained from PerkinElmer. FluorSave reagent was from Calbiochem. Rabbit polyclonal V-ATPase a2-subunit specific antibodies were previously described (Hurtado-Lorenzo et al. (Nat Cell Biol 8, 124-136, 2006)). Mouse monoclonal anti-GFP (B-2) and anti-6×His antibodies were purchased from Santa Cruz Biotechnology. Alexa 488-conjugated goat anti-mouse antibodies were obtained from Invitrogen. Horseradish peroxidase-conjugated sheep anti-mouse and anti-rabbit antibodies were purchased from GE Healthcare. Peptide synthesis resins and Fmoc-protected amino acids were purchased from EMD Chemicals. Dimethylformamide, N-methylyrrolidone, dichloromethane, piperidine, 2-(1H-benotrazol-1-yl) 1,1,3,3,-tetramethyl-hyuronium hexafluorophosphate, 1-hydroxy-benzo-triazol, diisopropylethyl-amine, and trifluoroacetic acid were purchased from American Bioanalytical. Diisopropyl carbodiimide, phenol, thioanisole, thiophenol, ethanedithiol, 4,6-diamidino-2-phenylindole (DAPI), M2 sepharose, and 3×FLAG peptide were from Sigma-Aldrich. Cytohesin inhibitor SecinH3 was a gift from Dr. Sylvain Bourgoin or from Calbiochem. A mini-extruder assembly was purchased from Avanti Polar Lipids. Guanosine-5′-triphosphate (GTP), guanosine diphosphate (GDP), and guanosine 5′-O-[γ-thio]triphosphate (GTPγS) were from Sigma-Aldrich, while [³⁵S]-GTPγS was from Perkin Elmer. Azolectin and phosphatidylglycerol (PG) were purchased from Sigma. Phosphatidylcholine (PC) was from Avanti Polar and phosphatidylinositol-4,5-bisphosphate (PIP₂) was from Matreya and Cayman Chemical.

cDNA Constructs—

GST-tagged cytohesin-2 (triglycine variant) and the Sec7 domain of human cytohesin-2 (61-252 aa) were cloned into a pGEX-6P-1 vector as described previously (Merkulova et al. (Am J Physiol Cell Physiol 300, C1442-1455, 2011)). Truncated cytohesin-2 lacking the coiled-coil domain (61-400aa)(CTH2₆₁₋₄₀₀) was cloned into a pET-28a(+) vector by restriction free cloning (van den Ent and Lowe (J Biochem Biophys Methods 67, 67-74, 2006)), including a His6 tag followed by a TEV protease site. Truncated [Δ17]Arf1 and [Δ13]Arf6 proteins were cloned into a pET28b vector. cDNA encoding full-length wild-type mouse V-ATPase a2-isoform (Toyomura et al. (J Biol Chem 275, 8760-8765, 2000)) was amplified using the PfuTurbo DNA Polymerase (Stratagene) and subcloned into NheI/AgeI restriction sites of the pEGFP-N1 vector (Clontech) in frame with C-terminal enhanced green fluorescent protein (EGFP).

Peptides Synthesis, Labeling and Purification—

All peptides were synthesized, purified by HPLC and analyzed by mass spectrometry in the MGH Peptide/Protein Core Facility as follows. Peptides were synthesized on an automatic peptide synthesizer (Applied Biosystems, Model 433A) by using the manufacturer's Fastmoc chemistry cycles for Fmoc solid-phase synthesis (Fields et al. (Pept Res 4, 95-101, 1991)). To render the V-ATPase derived a2N₁₋₁₇ peptide soluble and cell penetrable various versions of PEG or TAT modified peptides were synthesized and the final Fmoc group was removed. Peptides were further coupled at the N-terminus with either: i) biotin (in blue), ii) fluorescein isothiocyanate (FITC)(in green), iii) 5-carboxy-fluorescein (Fluor)(in green), or 5-carboxy-teramethylrhodamine (CTMR)(in red), using DIC/HOBt activation and an overnight reaction in the dark. The resin was then washed with dimethylformamide, dichloro-methane, and methanol three times each and vacuum dried. Peptides were cleaved from the solid support and de-protected using reagent K (TFA/phenol/thioanisole/water/ethanedithiol; 82.5/5.0/5.0/5.0/2.5 v/v) for 2.5 hours at room temperature (King et al. (Int J Pept Protein Res 36, 255-266, 1990)). Peptides were precipitated using cold methyl tertiary butyl ether (MTBE). The precipitate was washed three times in MTBE, dissolved in a solvent (0.1% trifluoroacetic acid in 20% Acetonitrile/80% water) followed by freeze drying. Peptide purification was performed on a semi-preparative system (Waters) using a Vydac C-18 reverse phase column and water/acetonitrile gradient consisting of 0.1% trifluoroacetic acid giving rise to >95% purity. All purified peptides were characterized by Ultra-high Pressure Liquid Chromatography (UPLC) and Matrix Assisted Laser Desorption/Ionization Mass Spectrometry (MALDI-MS). FITC-TAT was also purchased from Anaspec. Peptide stock solutions were prepared in water at 5 mM concentration and stored at −20° C.

Recombinant Protein Expression and Purification—

GST-tagged human cytohesin-2 was expressed in BL21DE3 cells (Stratagene) and purified as previously described (Merkulova et al. (Am J Physiol Cell Physiol 300, C1442-1455, 2011)). Cells were grown in LB at 37° C. Expression of recombinant protein was induced by addition of 1 mM isopropyl-β-D-thiogalactopyranoside (IPTG). Cells were disrupted by sonication in 50 mM sodium phosphate buffer (pH 7.0), 300 mM NaCl, 1 mM MgCl₂, 1 mM phenylmethyl-sulfonyl fluoride (PMSF), 0.5 mg/ml lysozyme, protease inhibitor cocktail (Roche). Lysate was centrifuged at 12,000×g for 12 minutes. Supernatant was centrifuged at 20,000×g for 60 minutes. Supernatant was applied to a PD10 column packed with glutathione Sepharose 4B beads (GE Healthcare). The column was washed with 50 mM sodium phosphate buffer (pH 7.0), 300 mM NaCl. GST-tagged human cytohesin-2 was eluted with 50 mM Tris-HCl (pH 7.7) and 10 mM reduced glutathione and then further separated by Superdex™ 200 HR 10/30 pre-packed column (GE Healthcare) using an AKTA Purifier system (GE Healthcare).

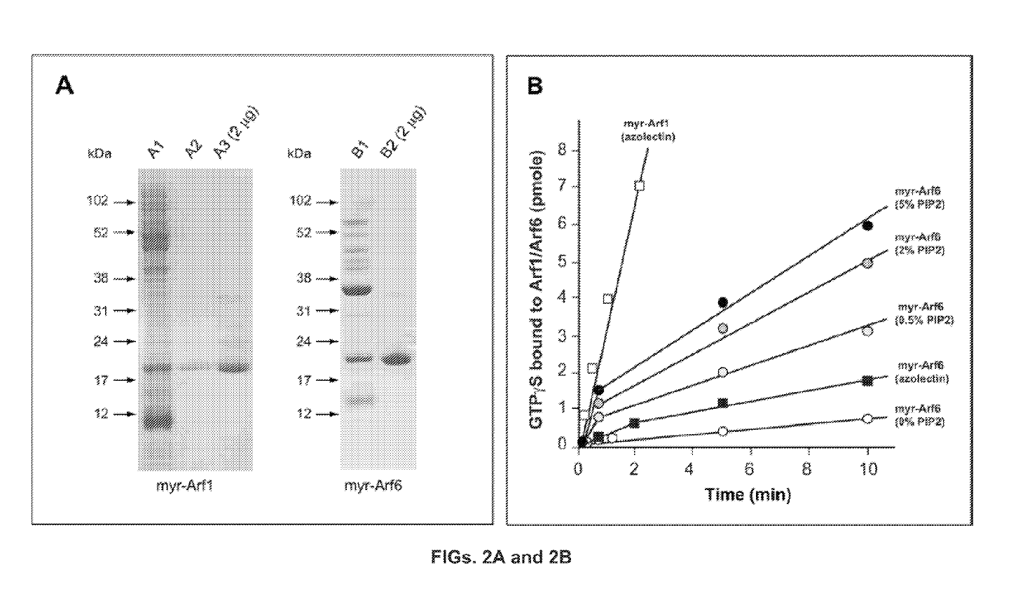

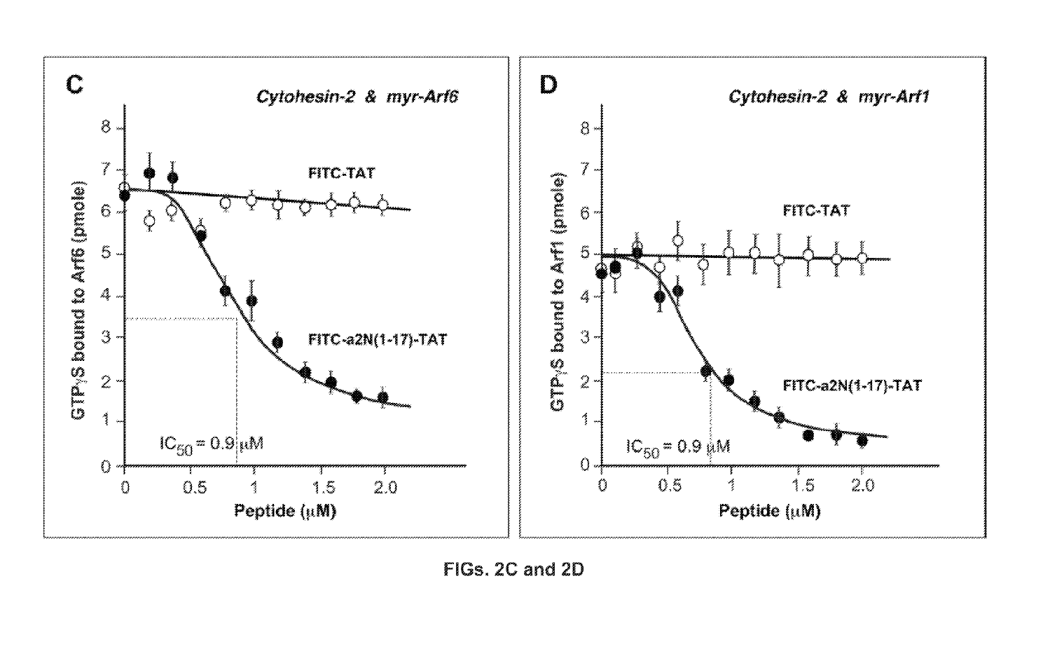

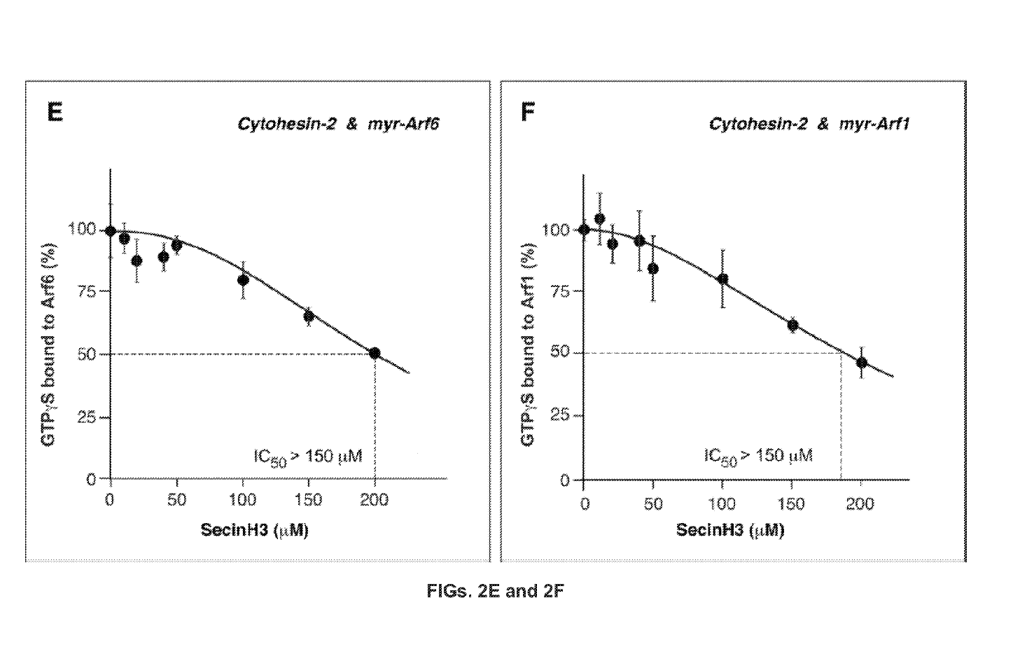

The Sec7 domain of human cytohesin-2 (61-252aa) was expressed in BL21/DE3 cells and purified by sequential chromatography on TALON beads (Clontech) and glutathione Sepharose 4B beads (GE Healthcare) as described previously (Merkulova et al. (Am J Physiol Cell Physiol 300, C1442-1455, 2011)). The GST-tag was cleaved using PreScission Protease™ (GE Healthcare). Myristoylated Arf1 and Arf6 were prepared as described previously (Ha et al. (Methods Enzymol 404, 164-174, 2005)). Arf1 was expressed in BL21/DE3 together with yeast N-myristoyltransferase. Cells were disrupted by sonication in 20 mM Tris-HCl (pH 8.0), 100 mM NaCl, 1 mM MgCl₂, 1 mM dithiothreitol (DTT) and 1 mg/ml lysozyme, protease inhibitor. The lysate was clarified by centrifugation at 100,000×g for 60 minutes at 4° C. The supernatant was loaded onto a 5-mL HiLoad Q column. Arf1 was eluted with 20 mM Tris-HCl (pH 8.0), 100 mM NaCl, 1 mM MgCl₂, 1 mM DTT, and 10% (v/v) glycerol. The fractions containing Arf1 were loaded onto the Sephacryl column. The fractions containing Arf1 were pooled. The protein solution was concentrated and buffer was exchanged to 20 mM Tris-HCl (pH 8.0), 3M NaCl, 1 mM MgCl₂, 1 mM DTT by ultrafiltration. Myristoylated Arf1 (myr-Arf1) was isolated using a phenyl Sepharose HP column. The column was developed in a gradient of 3M to 100 mM NaCl. NU-PAGE analysis shows >95% purity of myr-Arf1 (FIG. 2A).

Arf6 was expressed in BL21/DE3 together with yeast N-myristoyltransferase. Cells were disrupted by sonication in 20 mM Tris-HCl (pH 8.0 at 4° C.), 100 mM NaCl, 1 mM MgCl₂, 1 mM DTT, 10% glycerol, 1 mg/ml lysozyme, protease inhibitor. The lysate was centrifuged at 100,000×g for 60 minutes. The precipitate was resuspended in 20 mM Tris-HCl (pH 8.0 at 4° C.), 100 mM NaCl, 1 mM MgCl₂, 1 mM DTT, 10% glycerol, 1% Triton X-100. The lysate was clarified by centrifugation at 100,000×g for 60 minutes and 10 μM GDP was added to the supernatant. Myristoylated Arf6 (myr-Arf6) was precipitated in 35% saturation ammonium sulfate, dissolved in 20 mM Tris-HCl (pH8.0 at 4° C.), 25 mM NaCl, 1 mM MgCl₂, 1 mM DTT, 1% (w/v) Triton X-100, 10% (v/v) glycerol, 10 μM GDP and dialyzed against the same buffer. Myristoylated Arf6 was loaded on 5-ml HiTrap™ Q column and eluted with the same buffer. NU-PAGE analysis shows >95% purity of myr-Arf6 (FIG. 2A).

Truncated [Δ17]Arf1 and [Δ13]Arf6 proteins were expressed in BL21/DE3 cells grown in LB at 37° C. Expression of recombinant proteins was induced by addition of 1 mM IPTG. Cells were disrupted in 20 mM Tris-HCl (pH 8.0 at 4° C.), 100 mM NaCl, 1 mM MgCl₂, 1 mM DTT, 10% glycerol, 1 mg/ml lysozyme, protease inhibitor. Lysates were centrifuged at 12,000×g for 12 minutes. The supernatants were centrifuged at 100,000×g for 60 minutes and were separated by gel filtration (HiLoad 16/600 Superdex 75 pg). Purified [Δ17]Arf1 and [Δ13]Arf6 proteins were >95% pure according NU-PAGE analysis. In order to load [Δ17]Arf1 and [Δ13]Arf6 with GDP, both recombinant proteins were incubated with 10-fold GDP and 2 mM EDTA. Excess GDP was removed using a column packed with Sephadex G-25 μM.

GEF Activity Assays—

Two assays were used to determine the GDP/GTP-exchange activity of both full-length cytohesin-2 and Sec7 domain.

a) Radiolabel-Based Assay.

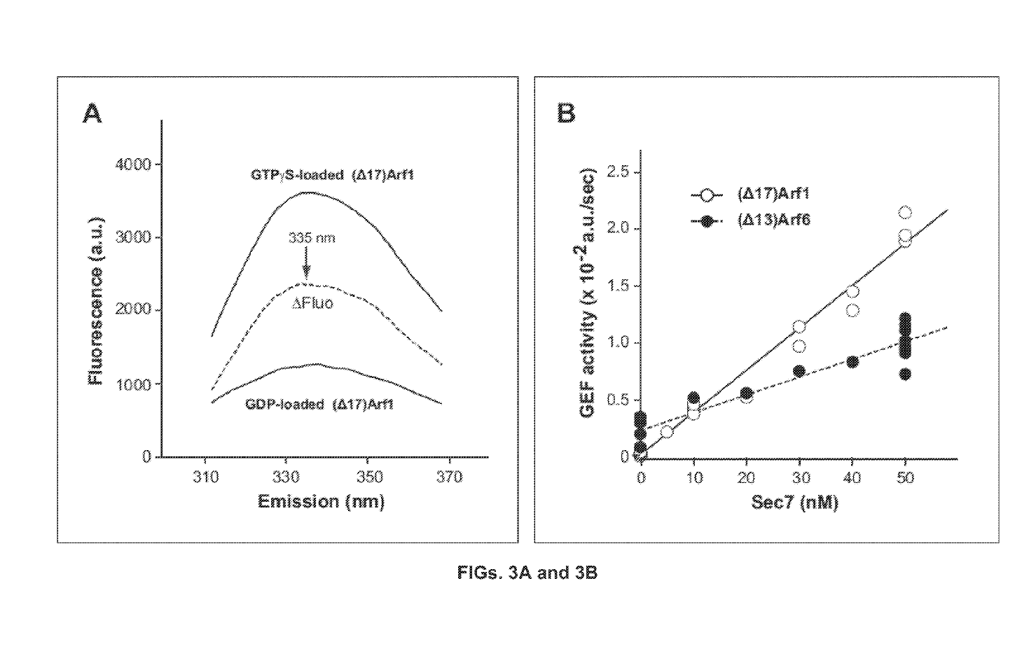

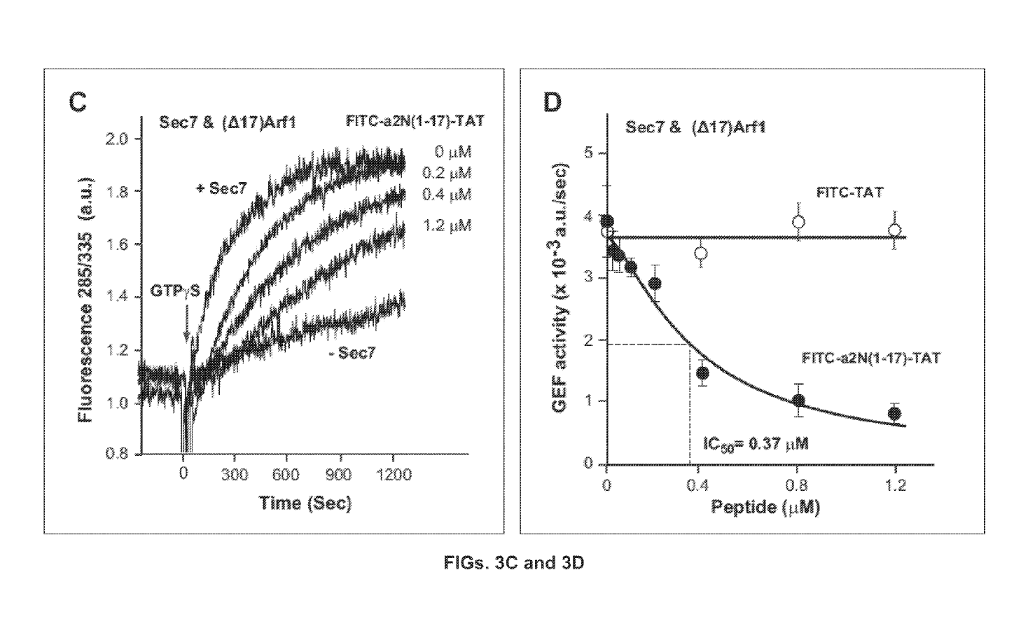

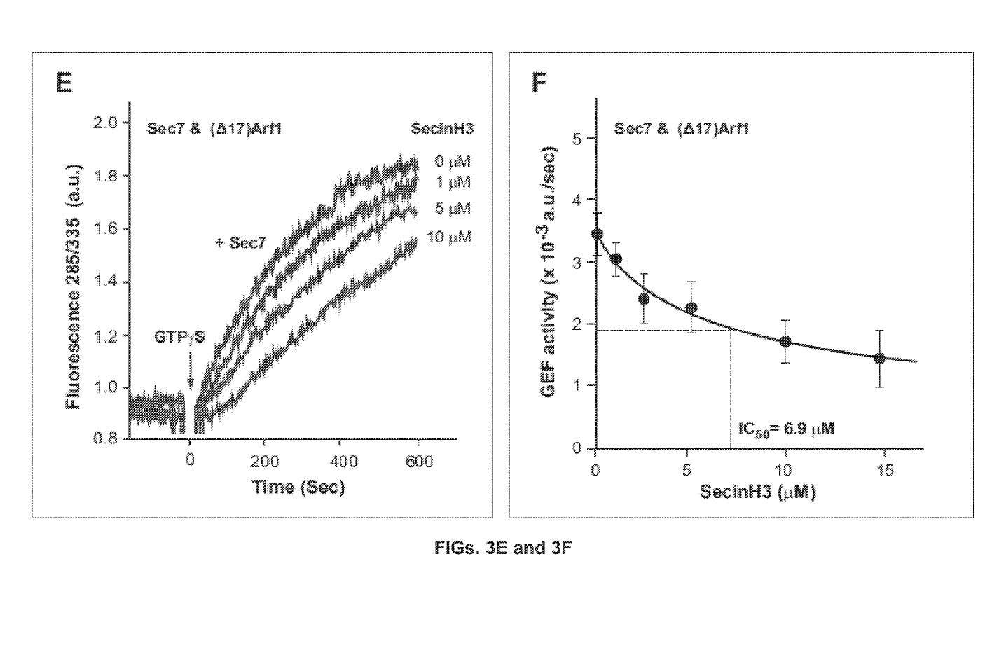

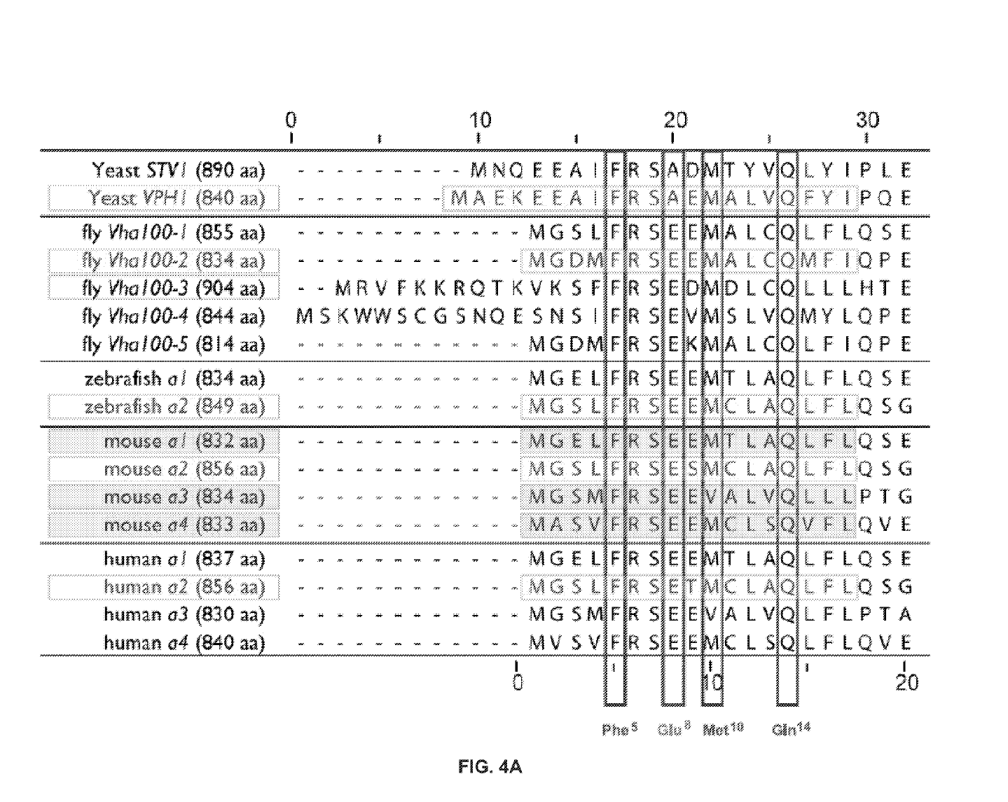

This assay allows the steady-state enzymatic GEF-activity analysis of full-length cytohesin-2 or Sec7 domain with Arf1 and Arf6 in the presence of PIP₂-containing liposomes (Santy et al. (Curr Biol 9, 1173-1176, 1999)). Phospholipid vesicles were prepared by the extrusion method (Macia et al. (Biochemistry 39, 5893-5901, 2000)). The lipids were dissolved in chloroform. A film of phospholipids was formed by evaporating chloroform and was resuspended in 50 mM HEPES, pH 7.5, and 1 mM DTT. The suspension was subjected to three freeze-thaw cycles and then passed through a 0.1 μm pore size polycarbonate filter (Millipore). For time-course measurements, recombinant myristoylated myr-Arf1 or myr-Arf6 were diluted to 1 μM in a buffer containing 50 mM HEPES, pH 7.5, 1 mM MgCl₂, 100 mM KCl, 1 mM DTT, and indicated lipid vesicles. Reactions were initiated by addition of 50 nM cytohesin-2 or Sec7 and 4 μM [³⁵S]-GTPγS and incubated at 37° C. At indicated times, aliquots (20 μl) were diluted into 2 ml of a buffer containing 20 mM HEPES, pH 7.5, 10 mM MgCl₂, 100 mM KCl to stop the reaction. Proteins were trapped on nitrocellulose filters. The radioactivity was quantified after washing the filters for three times with 2 ml of stop buffer. To test the effect of peptides, the 500 nM aliquots of cytohesin-2 or Sec7 were incubated with peptides (10-fold concentration shown on FIGS. 2 and 3) for 10 minutes before reactions were initiated. Reactions were continued for two minutes in the presence of 1.5 mg/ml azolectin with myr-Arf1. Alternatively reactions were carried out for 30 minutes in the presence of 1 mg/ml liposomes containing 65% (w/w) phosphatidylcholine (PC), 30% (w/w) phosphatidylserine (PS), and 5% (w/w) phosphatidylinositol-4,5-biphosphate (PIP₂) with myr-Arf6. To test the effects of SecinH3, the assays were performed as described above and SecinH3 was diluted with 4% DMSO. Titration with SecinH3 was performed up to a 200 μM concentration, due to its limited solubility in GEF-activity buffer.

b) Tryptophan Fluorescence-Based GTPγS Binding Assay.

In this assay, exchange is followed in real time. Truncated [Δ17]Arf1 and [Δ13]Arf6 were prepared as described (Viaud et al. (Proc Natl Acad Sci USA 104, 10370-10375, 2007)) for the assay. These are soluble in both GDP and GTP bound forms (myr Arf-GTP requires a hydrophobic surface for stability) allowing exchange to occur in the absence of lipid or detergent, which is desirable because lipid or detergent can scatter light and possibly confound interpretation of changes in fluorescence. To monitor tryptophan fluorescence, the fluorescent spectra of [Δ17]Arf1 in GDP-bound and GTPγS-bound forms were obtained at an excitation wavelength of 285 nm using a PTI spectrofluorimeter. The maximum difference was observed at 335 nm (FIG. 3A). Measurements were performed at 37° C. in 50 mM HEPES-KOH, 100 mM KCl, 1 mM MgCl₂, 1 mM DTT, 10 μM GTPγS, 1 μM [Δ17]Arf1 or [Δ13]Arf6 and 10 nM Sec7-domain. All titration experiments were started by addition of mixture containing GTPγS, Sec7, and a2N₁₋₁₇ peptide or SecinH3 inhibitor (as shown in FIG. 3). Data were fitted by single exponential curve and analyzed using Microsoft Excel.

Purification of Organelles and V-ATPase Holo-Complex—

Isolation of rat and mouse kidney proximal tubules and early endosomes was performed as previously described (Hurtado-Lorenzo et al. (Nat Cell Biol 8, 124-136, 2006); Liu et al. (Endocrinology 152, 1222-1233, 2011)). Yeast vacuolar membranes were isolated and the yeast V-ATPase holo-complex was purified by elution from the M2-sepharose matrix with 3×FLAG peptide as described previously (Benlekbir et al. (Nat Struct Mol Biol, In Press, 2012)).

PAGE and Western Blot Analysis—

Expression and integrity of endogenous full-length V-ATPase a2-subunit in purified kidney proximal tubules and early endosomes was analyzed by NU-PAGE and western blot analysis as previously described (Hurtado-Lorenzo et al. (Nat Cell Biol 8, 124-136, 2006)). Expression and integrity of recombinant full-length a2-EGFP in HeLa cells was also analyzed by NU-PAGE and western blotting. Briefly, overexpression of a2-EGFP was performed for one day using Lipofectamine 2000 transfection reagent. Total cell lysates were prepared, resolved by NU-PAGE and analyzed by western blotting using anti-GFP (B-2, 1:500) antibodies. Analysis of yeast V-ATPase integrity and its interaction with CTH2₆₁₋₄₀₀ was performed by SDS-PAGE and western blot analysis as previously described (Benlekbir et al. (Nat Struct Mol Biol, In Press, 2012)). Briefly, yeast V-ATPase immobilized on the M2-sepharose matrix was mixed with a large excess of recombinant CTH2₆₁₋₄₀₀ protein. Binding step was followed by four extensive washes to remove unbound CTH2₆₁₋₄₀₀ and contaminating proteins from the solubilized yeast membranes as previously described (Hurtado-Lorenzo et al. (Nat Cell Biol 8, 124-136, 2006)). Binding and co-purification of CTH2₆₁₋₄₀₀ with the V-ATPase holo-complex during elution with 3×FLAG peptide was analyzed by SDS-PAGE and confirmed by Western blotting with an anti-His6 antibody.

Immunocytochemistry and Confocal Microscopy Analysis—

Overexpression of full-length mouse a2-EGFP was performed for one day in MDCK cells grown on premium glass coverslips in six-well plates. Cells were rinsed in PBS and fixed in PBS containing 4% paraformaldehyde for 20 min. After three rinses in PBS, cells were permeabilized by treatment with 0.1% Triton X 100 in PBS for 10 min followed by blocking with 2% BSA in PBS for 1 hour. To increase the fluorescence signal of the overexpressed recombinant protein, the a2-EGFP was then detected with mouse monoclonal anti-GFP (B-2) antibodies. Cells were incubated for 18 hours at 4° C. with anti-GFP (B-2) antibodies (1:200 dilution in 2% BSA/PBS). After three PBS rinses, cells were incubated with Alexa488-conjugated goat anti-mouse antibodies (1:1,000 dilution in 2% BSA/PBS). Nuclei were counterstained with 0.75 μM of DAPI for 10 min. After three final PBS rinses, coverslips were mounted with FluorSave Reagent. Confocal microscopy was performed on a Nikon A1R laser scanning confocal microscope. Images were analyzed using Volocity v5 software.

Statistical Analysis—

Data are presented as mean values and error bars indicate the standard error of the mean (SEM). Statistical calculations were made using either Microsoft Excel or SigmaStat™ version 3.0 statistical software. The values of IC₅₀ were calculated using a four-parameter logistic function with Microsoft Excel.

Results

Signaling of a2-Subunit V-ATPase with Cytohesin-2 and Arf GTP-Binding Proteins.

To test the potential signaling between V-ATPase and cytohesin-2/Arf GTP-binding proteins, a GEF-activity assay was established for cytohesin-2 (wt) and myristoylated myr-Arf6 (wt) and myr-Arf1 (wt) GTP-binding proteins (FIG. 2A). Human Arf1 was co-expressed with yeast N-myristoyltransferase in E. coli BL21/DE3 strain. Recombinant myr-Arf1 was purified from the E. coli lysate (A1) using an AKTApurifier equipped with HiTrapQ, Superdex200 columns (A2), and Phenyl Sepharose HP column (A3). Human Arf6 was also co-expressed with yeast N-myristoyltransferase in E. coli BL21/DE3 strain. Recombinant myr-Arf6 was extracted from insoluble fraction using Triton X-100 (B1). The extract was precipitated by 35% ammonium sulfate and myr-Arf6 purified with HiTrapQ column (B2). This assay allows the steady-state enzymatic GEF-activity analysis of full-length cytohesin-2 or Sec7 domain with myristoylated myr-Arf1 and myr-Arf6 in the presence of PIP₂-containing liposomes (FIG. 2B). Several soluble, cell permeable and fluorescently labeled a2N₁₋₁₇ derived peptides were synthesized, which were used in the current GEF-activity and structural studies in vitro. After testing their solubility and cell permeability, the following V-ATPase derived peptides were used: i) FITC-a2N₁₋₁₇-TAT; ii) Biotin-a2N₁₋₁₇-TAT; and iii) a control FITC-TAT peptide. The GEF-activity assays demonstrated that FITC-a2N₁₋₁₇-TAT, but not FITC-TAT potently inhibits the enzymatic activity of cytohesin-2 (IC₅₀=0.9 μM) with both myr-Arf6 (FIG. 2C) and myr-Arf1 (FIG. 2D) as a substrate. In comparison, the SecinH3 small molecule, a known specific inhibitor of the cytohesin-2 Sec-7 domain, inhibited its activity with an IC₅₀>150 μM for myr-Arf6 (FIG. 2E) or IC₅₀>150 μM for myr-Arf1 (FIG. 2F). Thus, these studies show that a2N₁₋₁₇ peptide is significantly more potent (up to 150 times) inhibitor of cytohesin-2 than SecinH3.

To analyze whether the a2N₁₋₁₇ peptide inhibits the GEF-activity of cytohesin-2 via its direct and high affinity interaction with the Sec7 domain (Merkulova et al. (Biochim Biophys Acta 1797, 1398-1409, 2010)), a tryptophan fluorescence-based GEF-activity assay was also used. This assay allows real-time measurements of the GEF Sec7 domain-induced nucleotide exchange. [Δ17]Arf1 and [Δ13]Arf6, which are soluble in the GTP-bound form, were used as substrates, thereby eliminating the need for phospholipid or detergent in the assay. This approach eliminates the light scattering due to lipid or detergent that could confound interpretation of the fluorescence changes (Macia et al. (J Biol Chem 276, 24925-24930, 2001)). Activation of [Δ17]Arf1 or [Δ13]Arf6 from the GDP-bound form to the GTPγS-bound form was monitored by tryptophan fluorescence (emission/excitation wavelengths of 285/335 nm) (FIG. 3A). Since recombinant [Δ17]Arf1 and [Δ13]Arf6 were tagged with His₆ on the N-terminus, the affect that the tag has on the enzymatic reaction was first tested. The concentration dependence of GEF-activity of the Sec7 for both [Δ17]Arf1 and [Δ13]Arf6 demonstrated that the His6-tag did not interfere with the assay. The data also revealed the preferential activity of Sec7 with [Δ17]Arf1 than [Δ13]Arf6 as previously reported (FIG. 3B) (50,51). Using the purified Sec7 domain with truncated [Δ17]Arf1 as substrate, a direct and more potent inhibition of the Sec7 domain by the FITC-a2N₁₋₁₇-TAT peptide was found (IC₅₀=0.37 μM) (FIGS. 3C and 3D). In comparison, in this assay SecinH3 inhibits Sec-7/[Δ17]Arf1 with an IC₅₀=6.9 μM (FIGS. 3E and 3F). Taken together, these experiments demonstrate that V-ATPase derived a2N₁₋₁₇ peptide is a direct and potent inhibitor of the catalytic Sec7 domain of cytohesin-2.

Signaling Between V-ATPase, Cytohesin-2 and Arf GTP-Binding Proteins is an Evolutionarily Conserved Phenomenon.

Alignment of the first twenty amino acids of the N-terminal tail of V-ATPase a-subunit isoforms revealed that the interface-forming amino acids F₅ and Q₁₄ are completely conserved in all eukaryotes from yeast to humans (FIG. 4A). The interface-forming amino acid M₁₀ is also conserved from yeast to humans with only one substitution to V₁₀, which is found in the mouse and human a3-subunit V-ATPase (FIG. 4A). It is also noteworthy that while the E₈ residue is highly conserved, it has substitution to A₈ in yeast Stv1p and Vph1p (FIG. 4A). To test the potential signaling between other a-subunit (a1-, a3-, and a4-isoforms) of V-ATPase and cytohesin-2/Arf GTP-binding proteins, the corresponding a-subunit isoforms derived peptides were also synthesized (FIG. 4A). Additional enzymatic GEF-activity experiments demonstrated that these peptides are also very potent inhibitors of GEF-activity of cytohesin-2 Sec7 domain (FIGS. 4B to 4D). In these experiments the following IC₅₀ values were determined for a-isoform specific peptides: i) FITC-a1N₁₋₁₇-TAT (IC₅₀=1.0 μM) (FIG. 4B); ii) FITC-a3N₁₋₁₇-TAT (IC₅₀=0.5 μM) (FIG. 4C); and iii) FITC-a4N₁₋₁₇-TAT (IC₅₀=1.1 μM) (FIG. 4D). Thus, the data revealed the conserved character of signaling between all four a1-, a2-, a3-, and a4-subunit isoforms of V-ATPase with cytohesin-2 and Arf GTP-binding proteins.

Mammalian Cytohesin-2 Interacts with Yeast V-ATPase Holo-Complex Containing the Intact a-Subunit Isoform.

The conserved character of the signaling phenomenon in experiments showing binding of mammalian cytohesin-2 with yeast V-ATPase holo-complex was confirmed. First, a-subunit isoforms, which are involved in signaling with cytohesin-2, were verified to remain intact and functional in both mammalian and yeast V-ATPases. The kidney endosomal/lysosomal experimental system was used. Under physiological conditions in situ, endogenous a2-subunit isoform is not proteolytically processed and remains intact as a 100 kD protein in kidney proximal tubules (PT) and endosomes (Endo) (FIG. 5A). Western blot analysis did not reveal any additional low molecular weight proteins for either the a2-subunit (V_{O}-sector) or E-subunit (V₁-sector) of V-ATPase. Note that to provide better visualization of the lack of proteolytic cleavage of these proteins, Western blots of a2- and E-isoforms are presented as complete gels. These data indicate that in the mammalian kidney, the V-ATPase complex remains intact and competent for interaction and signaling with cytohesin-2 and Arf GTP-binding proteins. EGFP-tagged full-length a2-subunit isoform was also cloned and overexpressed. The data demonstrate that recombinant a2-EGFP remains intact as a 130 kD protein and does not undergo proteolytic cleavage (FIG. 5B). Importantly, overexpressed a2-EGFP is also correctly targeted to vesicular compartments in kidney cells (FIG. 5C).

Recently, the structure of the Saccharomyces cerevisiae eukaryotic V-ATPase was solved at 11 Å resolution by electron cryomicroscopy of protein particles on ice (Benlekbir et al. (Nat Struct Mol Biol, In Press, 2012)). This resolution was sufficient to resolve almost all of the individual subunits (FIG. 5D). In particular, the position of the intact a-subunit isoform (Vph1p homolog in yeast) in this V-ATPase (V₁V_{O}) holo-complex is shown in green and its epitope aN₁₋₁₇ is indicated by arrow (FIG. 5D). This V-ATPase holo-complex assembles with intact subunits (FIG. 5E), and allowed the study of its interaction with mammalian cytohesin-2. This preparation of the V-ATPase contained 13 different subunits indicated on the right, with the a-subunit isoform running as the intact full-length 116 kDa protein. Cytohesin-2 lacking its coiled-coil domain (CTH2₆₁₋₄₀₀) was used in this study. As previously reported, this form of cytohesin-2 does not dimerize and strongly interacts with the intact full-length mammalian a2-subunit isoform (Hurtado-Lorenzo et al. (Nat Cell Biol 8, 124-136, 2006)). Indeed, CTH2₆₁₋₄₀₀ binds intact yeast V-ATPase immobilized on M2-sepharose. Both CTH2₆₁₋₄₀₀ and V-ATPase holo-complexes are eluted together in an elution-step proportional manner (FIGS. 5F and 5G). Thus, taken together, these data confirm the evolutionarily conserved interaction and signaling of intact V-ATPase holo-complex with cytohesin-2 and Arf GTP-binding proteins.

The N-terminal epitope of the cytosolic tail of the a2-subunit of V-ATPase was previously identified as a major interacting site with cytohesin-2 (Merkulova et al. (Biochim Biophys Acta 1797, 1398-1409, 2010)). This epitope corresponds to the peptide a2N₁₋₁₇ which is formed by the first seventeen amino acids (MGSLFRSESMCLAQLFL) of the a2-subunit isoform. This V-ATPase epitope-forming a2N₁₋₁₇ peptide is crucial for signaling between V-ATPase and cytohesin-2, inhibiting enzymatic GDP/GTP-exchange activity of cytohesin-2 with both Arf1 and Arf6 as substrates. Moreover, this peptide strongly inhibits GEF-activity via its direct interaction with the catalytic Sec7 domain.

Interaction with cytohesin-2 is not only restricted to the a2-subunit isoform but also occurs with the three other a-subunit isoforms (a1, a3 and a4) of the V-ATPase (Merkulova et al. (Am J Physiol Cell Physiol 300, C1442-1455, 2011)). It is noteworthy, that these mammalian isoforms as well as two yeast a-subunit isoforms (Vph1p and Stv1p) function as full-length proteins and do not undergo proteolytic cleavage. Sequence alignment of the N-terminal epitope of a-subunit isoforms shows that the V-ATPase amino acids involved in interaction with Sec7 domain are highly conserved in all eukaryotes from yeast to humans. Accordingly, the enzymatic GEF-activity studies revealed that N-terminal peptides derived from all three a-subunit isoforms (a 1, a3, and a4) of the V-ATPase are potent inhibitors of cytohesin-2. Thus, these data reveal the conserved character of signaling between all four a1-, a2-, a3-, and a4-subunit isoforms of mammalian V-ATPase and cytohesin-2 (FIG. 6). These findings also indicate that signaling between V-ATPase and cytohesin-2 is a general cell biological phenomenon. Since a-isoforms are involved in targeting the V-ATPase to different cellular compartments (Marshansky and Futai (Curr Opin Cell Biol 20, 415-426, 2008)), the signaling between intact V-ATPase and cytohesin-2, Arf GTP-binding proteins could take place on endosomal, lysosomal, Golgi, or plasma membranes among others (Marshansky and Futai (Curr Opin Cell Biol 20, 415-426, 2008)).

Four novel a-isoform-specific V-ATPase derived peptides (a1N₁₋₁₇, a2N₁₋₁₇, a3N₁₋₁₇ and a4N₁₋₁₇) that are potent inhibitors of cytohesin-2 GEF-activity in vitro have been identified and characterized. The IC₅₀ values for all four a-subunit isoform specific peptides were determined (FIG. 6). The interaction competent amino acids (F₅ and Q₁₄) are completely conserved in all eukaryotic a1-, a2-, a3-, and a4-isoforms, and their corresponding specific peptides show similar IC₅₀˜1.0 μM values. The structural interface of signaling between the V-ATPase and cytohesin-2 provides novel potent anti-cytohesin-2 pharmaceuticals/peptides. In particular, these novel isoform-specific pharmaceuticals and drugs could be used to prevent the cytohesin-2 dependent activation of EGFR/ErbB receptors in order: i) to treat variety of cancers (Hafner et al. (Nat Protoc 3, 579-587, 2008); Bill et al. (Cell 143, 201-211, 2010); Bill et al. (PLoS ONE 7, e41179, 2012)); and ii) to treat or reduce a risk of developing diabetic nephropathy in type 1 diabetes (Sandholm et al. (PLoS Genet 8, e1002921, 2012); Boger and Sedor (PLoS Genet 8, e1002989, 2012); Panchapakesan et al. (Clin Exp Pharmacol Physiol 38, 84-88, 2011)).

### Example 2

### Regulation of Macropinocytosis Pathway and Cell Proliferation by Cytohesin-2

Reagents and cDNA Constructs—

If not otherwise specified, all reagents including inhibitors bafilomycin A₁, amiloride, 5-N,N-dimethylamiloride hydrochloride (DMA), 5-(N-ethyl-N-isopropyl)amiloride (EIPA), RITC-dextran, and 3-(4,5-dimethylthiazol-2-yl)-2,5-diphenyltetrazolium bromide (MTT) were purchased from Sigma-Aldrich. All buffers, NU-PAGE gels and DAPI were from Invitrogen. Peptide synthesis resins and Fmoc-protected amino acids were purchased from EMD Chemicals. Dimethylformamide, N-methylyrrolidone, dichloromethane, piperidine, 2-(1H-benotrazol-1-yl) 1,1,3,3-tetramethylhyuronium hexafluorophosphate, 1-hydroxybenzotriazol, diisopropylethylamine, and trifluoroacetic acid were purchased from American Bioanalytical. Diisopropyl carbodiimide, phenol, thioanisole, thiophenol and ethanedithiol were from Sigma. Fluorescein isothiocyanate, 5-carboxy-fluorescein, and 5-carboxy-teramethyl-rhodamine were from Anaspec. Bovine serum albumin conjugated with either Alexa555 (albumin-Alexa555), Alexa594 (albumin-Alexa594) or DQ-Red (DQ-Red BSA) were from Invitrogen, while FITC-TAT was from Anaspec. Dulbecco's modification of Eagle's medium (DMEM) was from Mediatech. Fetal bovine serum (FBS) was from Atlanta Biologicals. Cytohesin inhibitor SecinH3 was from Calbiochem or gift from Dr. Sylvain Bourgoin and dissolved in DMSO. Rab5-EGFP and Rab7-EGFP plasmids were provided by Dr. Christian Reinecker and were previously described (Hurtado-Lorenzo et al. (Nat Cell Biol 8, 124-136, 2006)). LAMP1-mGFP was a gift from Dr. Esteban C. Dell'Angelica (Falcon-Perez et al. (J Cell Sci 118, 5243-5255, 2005)). EEA1-GFP was provided by Dr. Silvia Corvera (Leonard et al. (J Cell Sci 121, 3445-3458, 2008)).

Peptides Synthesis, Labeling and Purification—

All peptides were synthesized, purified by HPLC and analyzed by mass spectrometry in the MGH Peptide/Protein Core Facility as follows. Peptides were synthesized on an automatic peptide synthesizer (Applied Biosystems, Model 433A) by using the manufacturer's Fastmoc chemistry cycles for Fmoc solid-phase synthesis (Rishikesan et al. (Mol Membr Biol 25, 400-410, 2008)). To render the V-ATPase derived a1N₁₋₁₇; a2N₁₋₁₇; a3N₁₋₁₇, and a4N₁₋₁₇ peptides soluble and cell penetrable, TAT modified peptides were synthesized. Peptides were further coupled at the N-terminus with either: i) fluorescein isothiocyanate (FITC), ii) 5-carboxy-fluorescein (Fluor), or iii) 5-carboxy-teramethylrhodamine (CTMR) using DIC/HOBt activation and an overnight reaction in the dark. The resin was then washed with dimethylformamide, dichloro-methane, and methanol three times each and vacuum dried. Peptides were cleaved from the solid support and de-protected using reagent K (TFA/phenol/thioanisole/water/ethanedithiol; 82.5/5.0/5.0/5.0/2.5 v/v) for 2.5 hours at room temperature (Merkulova et al. (Am J Physiol Cell Physiol 300, C1442-1455, 2011)). Peptides were precipitated using cold methyl tertiary butyl ether (MTBE). The precipitate was washed three times in MTBE, dissolved in a solvent (0.1% trifluoroacetic acid in 20% Acetonitrile/80% water) followed by freeze drying. Peptide purification was performed on a semi-preparative system (Waters) using a Vydac C-18 reverse phase column and water/acetonitrile gradient consisting of 0.1% trifluoroacetic acid giving rise to >95% purity. All purified peptides were characterized by Ultra-high Pressure Liquid Chromatography (UPLC) and Matrix Assisted Laser Desorption/Ionization Mass Spectrometry (MALDI-MS).

Peptide Cell Permeability and Targeting Assay—

HeLa or MTC cells were grown to 50% confluency in DMEM with 10% FBS on 4-well Culture Slide (BD). Cells were incubated with Ringer's buffer (10 mM HEPES, 122.5 mM NaCl, 5.4 mM KCl, 0.8 mM MgCl₂, 1.2 mM CaCl₂, 1.0 mM NaH₂PO₄, 5.0 mM glucose) (pH 7.4) containing 5 μM FITC-TAT or FITC-a2N₁₋₁₇-TAT for 10 minutes at 37° C. Cells were then washed three times with Ringer's buffer (pH7.4) followed by 20 minutes fixation in 4% paraformaldehyde. Confocal microscopy analysis was performed using a Zeiss Radiance 2000 confocal microscope system controlled by LaserSharp 2000 software, and Z-series of the entire cells were acquired. Cells were reconstructed and analyzed using Volocity v.5 software (Improvision).

Cell Population Albumin Endocytic Uptake Assay—

Quantitative endocytic uptake of albumin-Alexa555 and RITC-dextran was performed using a cell population assay as previously described (Hurtado-Lorenzo et al. (Nat Cell Biol 8, 124-136, 2006)). HeLa cells were seeded on 48-well plates (5,000 cells/well) and grown for 5 days. MTC cells were seeded on 48-well plates (5,000 cells/well) and grown for 7 days until polarized, followed by starvation for 2 days in serum-free DMEM medium. Cells were incubated in 200 μl of Ringer buffer (pH 7.4) containing either 200 μg/ml albumin-Alexa555 or 500 μg/ml RITC-dextran in the presence of 5 μM FITC-TAT or 5 μM FITC-a2N₁₋₁₇-TAT peptides. As indicated, cells were also treated with 1 μM bafilomycin A₁, 1 mM amiloride, 100 μM DMA or 100 μM EIPA. At the indicated times, cells were washed with Ringer's buffer (pH 6.0) for three times and were permeabilized in 20 mM MOPS (pH 7.0), 0.1% Triton X-100. The fluorescence was measured using a DTX 880 Microplate Reader (Beckman) with 535-nm excitation and 595-nm emission filters (FIGS. 7, 8, and 12).

Cell Population Albumin Degradation Assay—

Quantitative protein degradation assay was performed using DQ-Red BSA. MTC cells were seeded on 48-well plate (5,000 cells/well) and grown for 7 days until being polarized, followed by starvation for two days in serum-free DMEM medium. Cells were washed with Ringer buffer (pH 6.0) for three times and with Ringer buffer (pH 7.4) once. Cells were then incubated in 200 μl of Ringer buffer (pH 7.4) containing as indicated: 5 μM FITC-TAT, 5 μM FITC-a2N₁₋₁₇-TAT peptide, 1 μM baflomycin A₁, 20 mM NH₄Cl, 1 mM amiloride or 100 μM DMA. DQ-Red BSA was added to a final concentration of 10 μg/ml. The fluorescence was estimated using a DTX 880 Microplate Reader (Beckman) with 590-nm excitation and 625-nm emission filters every 3 minutes. The 48-well plate was kept at 37° C. in the microplate reader (FIG. 11).

Pulse-Chase Experiments and Live Time-Lapse Confocal Fluorescence Imaging—

Live cell imaging of MTC cells was performed using time-lapse confocal fluorescent imaging as previously described (Hurtado-Lorenzo et al. (Nat Cell Biol 8, 124-136, 2006)). MTC cells were seeded on FluoroDish (World Precision Instruments, Inc) (25,000-100,000 cells/dish) and grown to about 50% confluency. To label the organelles of the endocytic pathway in vivo, MTC cells were transfected using lipofectamine 2000 (Invitrogen) with 5 μg of the plasmids: i) EEA1-GFP or Rab5-EGFP (early endosomal markers); ii) Rab7-EGFP (late endosomal marker), and iii) LAMP1-mGFP (lysosomal marker). After two days, the cells were washed for three times with Ringer's buffer (pH 6.0) followed by one wash with Ringer's buffer (pH 7.4) at 37° C., and i) pulsed for 15 min with 5 μM FITC-TAT and 100 μg/ml albumin-Alexa594: ii) pulsed for 15 min with 5 μM FITC-a2N₁₋₁₇-TAT and 100 μg/ml albumin-Alexa594; or iii) pre-incubated in the presence or absence of 1 μM bafilomycin A₁ and pulsed with 100 μg/ml albumin-Alexa594. The MTC cells transfected with EEA1-GFP, Rab5-EGFP, Rab7-EGFP and LAMP1-GFP plasmids were pulsed for 6-15 min with 5 μM of CTMR-a2N₁₋₁₇-TAT peptide. After the pulse, cells were washed either by perfusion or rinsing with Ringer's buffer (pH 7.4) and incubated in an environmental chamber (37° C., 5% CO₂) coupled to a Nikon inverted microscope. Videos were acquired with a spinning-disc confocal imaging system Ultra View RS (Perkin Elmer). FIGS. 9 and 10 show frames from these movies.

Image Analysis and Tracking of Endocytic Vesicles—

Dynamics of the movement of early endosomes, late endosomes and lysosomes labeled with either: i) albumin-Alexa594; ii) FITC-a2N₁₋₁₇-TAT and albumin-Alexa594; or iii) CTMR-a2N₁₋₁₇-TAT and transfected EEA1-GFP markers were tracked with Particle Tracker PlugIn (Merkulova et al. (Biochim BiophysActa 1797, 1398-1409, 2010)) for ImageJ (NIH). The trajectories that appeared for over 30 frames were further analyzed. The two-dimensional displacement between consecutive frames was calculated from changes in X and Y position coordinates and speed was calculated by dividing the displacement by time. The two-dimensional displacements between all possible combinations of frames were also calculated and the longest distance was defined as “maximum distance” of the trajectory. The speeds were averaged for 11 consecutive frames and the fastest speed was defined as “maximum speed” of the trajectory.

Cell Proliferation Assays—

First, MTT proliferation assay was carried out using cell growth determination kit according to manufacturer instructions (Sigma). Human NSCLC A549 cells which are expressing wild-type EGFR with KRas-G12S activation mutation were studied in these experiments. Cells were seeded onto a 96-well plate (3,000 cells per well) and grown at 37° C. and 5% CO₂ in RPMI-1640, 10% FBS. After one day, the cells were treated with peptides or SecinH3 diluted in RPMI-1640, 1% FBS. Medium was changed daily for three days, and cells were stained with MTT and DAPI when indicated. Absorbance of cells stained by MTT was estimated between 595 and 620 nm using a DTX 880 Microplate Reader (Beckman).

Second, direct cells counting assays were also performed as follows. Microscopic images of 0.317 mm² area each well were acquired for MTT stained cells and cells were counted using ImageJ (NIH). Images of cell stained with MTT were inverted and filtered by Gaussian blur filter and cells were counted using Particle Tracker PlugIn for ImageJ (Sbalzarini et al. 2005 J Struc Biol, 151, 182). Alternatively, the cells were stained with DAPI and microscopic images of whole wells were acquired using DeltaVision system (Applied Precision). Cells were counted using Particle Tracker PlugIn for ImageJ as above (FIG. 13).

Statistical Analysis—

Data are presented as mean values and error bars indicate the standard error of the mean (SEM). Statistical calculations were made using either Microsoft Excel or SigmaStat™ version 3.0 statistical software. Homogeneity of variance was assessed by Bartlett's test, and p values were obtained from Dunnett's test. Asterisks indicate values that are significantly different (p<0.05) relative to control cells.

Results

Four V-ATPase derived peptides (a1N₁₋₁₇, a2N₁₋₁₇, a3N₁₋₁₇, and a4N₁₋₁₇) as a potent inhibitors of the enzymatic GDP/GTP-exchange activity of cytohesin-2 were rendered fluorescently labeled and cell-permeable by attaching a FITC- or CMTR-moiety and TAT (YGRKKRRQRRR; SEQ ID NO:33) sequence, respectively. These synthetic peptides were used as potent inhibitors of cytohesin-2 in vivo, to study their role in regulation of endocytic pathways and cell proliferation.

V-ATPase-Derived Anti-Cytohesin Peptide Specifically Targeted and Accumulated in Vesicular Endocytic Compartments.—

To evaluate the cell biological action of novel anti-cytohesin peptides, the FITC-a2N₁₋₁₇-TAT peptide was studied to confirm that it is cell-penetrable and could accumulate in specific intracellular compartments. Targeting experiments with MTC (FIGS. 7A and 7B) and HeLa cells demonstrated that labeling with either FITC- or CTMR- and attachment of a TAT-sequence to the a2N₁₋₁₇ peptide indeed renders it cell permeant and applicable for live-cell immunofluorescence analysis. It is noteworthy, that this anti-cytohesin FITC-a2N₁₋₁₇-TAT peptide is targeted to specific vesicular endocytic compartments (FIG. 7A) and displays up to three hours linear uptake and accumulation in cells (FIG. 7C). In contrast, the biologically inactive FITC-TAT peptide, used as a control, displays a predominantly cytosolic distribution (FIG. 7B) and does not accumulate in cells over time (FIG. 7C).

V-ATPase-Derived Anti-Cytohesin Peptide Strongly Stimulate Uptake of Different Endocytic Markers.—

To study the biological role of FITC-a2N₁₋₁₇-TAT peptide in the regulation of the endocytic pathways, its effect using a cell population endocytic uptake assay was tested as previously described (Hurtado-Lorenzo et al. (Nat Cell Biol 8, 124-136, 2006)). Uptake of albumin-Alexa555 by kidney proximal tubules in situ and MTC cells takes place predominantly via the megalin/cubilin-receptor mediated endosomal/lysosomal protein degradation pathway under non-stimulated conditions. The pathway is regulated by V-ATPase driven endosomal acidification and interaction between the a2-subunit isoform and cytohesin-2 (Hurtado-Lorenzo et al. (Nat Cell Biol 8, 124-136, 2006)). Surprisingly, very potent activation of albumin-Alexa555 uptake by the FITC-a2N₁₋₁₇-TAT peptide was found in cell-population experiments. In particular, there was a dramatic (20-fold) increase in uptake of albumin-Alexa555 during the first 60 min followed by a plateau over the next 60-180 min (FIG. 8A). Control FITC-TAT peptide had no effect on the gradual and linear uptake of albumin-Alexa555 in non-treated control MTC cells (FIG. 8A). Similarly, treatment of MTC cells with FITC-a2N₁₋₁₇-TAT peptide also significantly increased uptake of fluid-phase marker RITC-dextran (FIG. 8B). Importantly, that this endocytosis stimulatory effect of V-ATPase derived anti-cytohesin peptide was not cell specific and was also observed in HeLa cells. Indeed, treatment of HeLa cells with FITC-a2N₁₋₁₇-TAT, but not FITC-TAT, significantly stimulated an uptake of both albumin-Alexa555 (FIG. 8C) and RITC-dextran (FIG. 8D) endocytic tracers.

Cell-Permeable Anti-Cytohesin Peptide Gives Rise to Cell Shape Remodeling and Activation of the Macropinocytosis Pathway.—

To uncover the cell biological mechanism of this stimulatory action of V-ATPase anti-cytohesin peptide, a single-cell analysis of the function of specific fluorescently labeled endocytic compartments was also applied, as previously described (Hurtado-Lorenzo et al. (Nat Cell Biol 8, 124-136, 2006)). In these experiments, CMTR-labeled peptide (CTMR-a2N₁₋₁₇-TAT) was used to study early endocytic event in MTC cells. Early endosomal compartments were labeled by in vivo transfection with either EEA1-EGFP or Rab5-EGFP and effect of peptide was studied in live-cells using time-lapse confocal-microscopy analysis. Frames from the movie are shown in FIGS. 9A, 9A₁, and 9A₂. Three modes of CTMR-a2N₁₋₁₇-TAT action were found on early stages of the endocytic pathway: i) cell-site specific accumulation of the peptide in the large and dynamic vacuolar compartments, which were only partially colocalized with early endosomes (FIG. 9A, arrowheads), ii) penetration and appearance of the peptide in the cytosol of MTC cells (FIG. 9A, insert A₁), and iii) uniform attachment of peptide at the surface of the plasma membrane (FIG. 9A, insert A₂, arrows). The appearance of peptide in the cytosol is seen only at specific sites (FIG. 9A, insert A₁) and does not occur over the entire cell surface (FIG. 9A, insert A₂). This is a very rapid early event accompanied by site-specific and dramatic cell-shape remodeling (FIG. 9A, inserts A₁). The movement dynamics of the total population of EEA1-EGFP positive early endosomes were observed in live-cells using confocal-microscopy time-lapse movies (FIGS. 9A₁ and 9A₂). Analysis of vesicular movement demonstrated a significant increase in moving speed (FIG. 9B) and moving distance (FIG. 9C) of early endosomes upon appearance of CTMR-a2N₁₋₁₇-TAT peptide in the cytosol of MTC cells. This phenotype of the formation of large vacuolar compartments, accompanied with cell-shape remodeling, which is also coupled with acceleration of early endosomal function, is consistent with cell-site specific activation of macropinocytosis pathway. This novel cell biological action of V-ATPase derived anti-cytohesin peptide may explain the robust (20-fold) increase in uptake of albumin-Alexa555 and RITC-dextran by MTC and HeLa cells (FIG. 8).

Cell-Permeable Anti-Cytohesin Peptide Accumulates in Endosomal/Lysosomal Compartments and Slows-Down Vesicular Movement.—

To study the effect of the V-ATPase-derived anti-cytohesin peptide on later stages of the protein degradation pathway, a live-cell confocal microscopy approach was also used. In these experiments, late endosomes and lysosomes were labeled in vivo by transfection with Rab7-EGFP and LAMP1-mGFP, respectively, and trafficking of CTMR-a2N₁₋₁₇-TAT peptide in these compartments was followed using time-lapse imaging. These experiments demonstrated the specific targeting and localization of this peptide in late endosomes (FIG. 10A) and lysosomes (FIG. 10B) of the protein degradation pathway. Similar pulse-chase analysis of the FITC-a2N₁₋₁₇-TAT peptide also revealed its targeting and localization in albumin-Alexa594 positive late endosomal/lysosomal compartments (FIG. 10C).

An additional experiments examining simultaneous uptake of albumin-Alexa594 and FITC-a2N₁₋₁₇-TAT for 90 min also revealed their significant colocalization in late endosomal/lysosomal compartments. Importantly, quantitative analysis without (FIG. 10D) and with peptide treatment (FIG. 10E) demonstrated that the peptide significantly decreased the speed (FIG. 10F) and distance moved (FIG. 10G) of the late endosomal/lysosomal compartments.

Treatment of MTC cells with a specific V-ATPase inhibitor bafilomycin A₁ was previously demonstrated to strongly suppress function of this pathway due to impaired vesicular trafficking between early and late endosomal compartments (Hurtado-Lorenzo et al. (Nat Cell Biol 8, 124-136, 2006)). Here, its action could be additionally attributed to the inhibition of motility of albumin-positive late endosomal/lysosomal compartments. Single-cell analysis was also applied and movement of the total population of albumin-Alexa594 positive vesicles was analyzed (FIGS. 10H and 10I) Similar to the anti-cytohesin peptide, bafilomycin A₁ had a time-dependent inhibitory effect on the moving distance (FIG. 10J) and 20% inhibitory effect on speed of the albumin-Alexa594 positive vesicles at the late stages of trafficking within endosomal/lysosomal protein degradation pathway.

Differential Action of V-ATPase, NHE Inhibitors and Anti-Cytohesin Peptides on Albumin Degradation in Endosomal/Lysosomal Compartments.—

In order to further asses the cell biological mechanism of this phenomenon the effect of V-ATPase, NHE inhibitors, and anti-cytohesin-peptides on degradation of DQ-Red-BSA in endosomal/lysosomal compartments of MTC cells was studied. It is noteworthy that in control non-stimulated conditions, the V-ATPase and NHE3 inhibitors have opposite effects on the endosomal/lysosomal degradation of DQ-Red-BSA (FIG. 11). Using this cell-population assay, bafilomycin A₁ (Baf) (FIGS. 11A and 11E) and NH₄Cl (FIG. 11E) strongly inhibited degradation of DQ-Red-BSA protein in endosomal/lysosomal compartments. In contrast, the NHE inhibitors, DMA (FIGS. 11B and 11E) and amiloride (FIG. 11E), significantly stimulated degradation of DQ-Red-BSA via this pathway. In contrast, treatment of MTC cells with either FITC-TAT (FIGS. 11C and 11E) or FITC-a2N₁₋₁₇-TAT (FIGS. 11D and 11E) peptides did not have any effect on degradation of DQ-Red-BSA.

Differential Action of V-ATPase and NHE Inhibitors on Receptor-Mediated and Macropinocytosis Pathways in MTC Cells.—

The effect of V-ATPase and NHE inhibitors on the uptake of endocytic tracers (albumin-Alexa555 and RITC-dextran) via receptor-mediated and macropinocytosis pathways in MTC cells was tested. First, single-cell analysis and previous studies on the inhibitory action of bafilomycin A₁ on receptor-mediated uptake of albumin-Alexa555 in control non-stimulated conditions were confirmed (FIG. 12A) (Hurtado-Lorenzo et al. (Nat Cell Biol 8, 124-136, 2006)). In contrast, this specific V-ATPase inhibitor had no effect on the uptake of albumin-Alexa555 by macropinocytosis pathway (FIG. 12A). However, it is noteworthy that specific NHE inhibitors DMA and EIPA had opposite effects on the function of these two pathways (FIG. 12B). Treatment of cells with DMA (FIG. 12B) or EIPA does not affect uptake of albumin-Alexa555 via receptor-mediated pathway in control non-stimulated conditions (FIG. 12B). However, both DMA (FIG. 12B) and EIPA could inhibit uptake of albumin-Alexa555 via anti-cytohesin peptide-induced macropinocytosis pathway (FIG. 12B). Importantly, uptake of fluid phase marker RITC-dextran in both control and peptide-stimulated conditions was insensitive to the treatment of MTC cell with inhibitors of V-ATPase (FIG. 12C) and NHE (FIG. 12D). Similar results were also obtained with HeLa cells.

V-ATPase Derived Anti-Cytohesin Peptides are Potent Inhibitors of Cell Proliferation.—

To assess the effect of V-ATPase derived anti-cytohesin peptides on cell proliferation, human NSCLC A549 cells that express wild-type EGFR with KRas-G12S activation mutation were used. First, the effect of SecinH3 was tested and confirmed the previous studies of anti-proliferative action of this anti-cytohesin inhibitor (FIGS. 13B and 13C). All V-ATPase derived anti-cytohesin peptides (a1N₁₋₁₇, a2N₁₋₁₇, a3N₁₋₁₇, and a4N₁₋₁₇) are also very potent inhibitors of proliferation of the human lung cancer A549 cells (FIGS. 13A to 13C).

Endocytic vesicular trafficking is a fundamental cellular process that is used by eukaryotic cells to communicate with their external milieu and internalize a variety of macromolecules and microorganisms. It is also crucial for the retrieval, signaling and recycling of receptors localized at the plasma membrane (FIG. 14) (Doherty and McMahon (Annu Rev Biochem 78, 857-902, 2009)). The best studied is the clathrin-dependent endocytosis (CDE) pathway, which mediates internalization of EGFR/ErbB, megalin/cubilin and transferrin receptors among many others (FIG. 14, right). However during the last decade, various clathrin-independent endocytosis (CIE) pathways including Arf6-dependent endocytosis and macropinocytosis pathways were identified (FIG. 14, left) (Doherty and McMahon (Annu Rev Biochem 78, 857-902, 2009); Mayor and Pagano (Nat Rev Mol Cell Biol 8, 603-612, 2007)). In HeLa and COS cells the Arf6-dependent CIE pathway operates constitutively and intersects in early endosomes with the CDE pathway (Donaldson et al. (Cell Signal 21, 1-6, 2009); Grant and Donaldson (Nat Rev Mol Cell Biol 10, 597-608, 2009)). Modulation of overexpressed Arf6 activity in these cells promotes a switch from a constitutive to a stimulated macropinocytosis pathway (Donaldson et al. (Cell Signal 21, 1-6, 2009); Grant and Donaldson (Nat Rev Mol Cell Biol 10, 597-608, 2009)). In particular, overexpression of dominant-active Arf6-Q67L mutant inhibits recycling of fluid phase tracers and leads to an accumulation of large vacuolar membranes representing trapped macropinosomes. In these studies, an elaborate interplay between cytohesin-2/EFA6 GDP/GTP-exchange factors and Arf6/Arf1 GTP-binding proteins in regulation of stimulated macropinocytosis pathway was proposed (Donaldson et al. (Cell Signal 21, 1-6, 2009); Grant and Donaldson (Nat Rev Mol Cell Biol 10, 597-608, 2009)).

### Example 3

### High Glucose Levels (“Glucose Toxicity”) During Early Stages of Type 1 Diabetes Triggers Up-Regulation of Expression of Cytohesin-2, V-ATPase and Megalin Receptors in Endosomal/Lysosomal Protein Degradation Pathway of Mouse Kidney Proximal Tubules (PT) Epithelial Cells

Dysregulation of signaling between V-ATPase, cytohesin-2, aldolase, EGFR/Erb, and megalin-receptors in PT may have important implications for the pathophysiology and development of early (1-4 weeks) stages of diabetic nephropathy during type 1 diabetes. This Example was performed using non-obese diabetic (NOD) mice as a model of human Type 1 diabetes.

Briefly, NOD mice were purchased from The Jackson Laboratory and onset of diabetes was determined by daily measurement of tail-blood glucose levels. Mice showing blood glucose levels higher than 300 mg/dL for two consecutive days were considered to be diabetic. Mice were separated into three diabetic NOD groups (0, 14, and 28 days) and three control groups (0, 14, and 28 days) with three mice in each group. The development of type 1 diabetes in a colony of NOD mice is shown in FIG. 15, and a summary of the control and diabetic groups is shown in FIG. 16.

Laser-cut microdissection (LCM) is a powerful technique for isolation of different cell types from complex tissues. We developed a method to identify “albumin-positive” proximal tubules in vivo and to isolate these tubules by LCM. This approach was successfully used in previous studies of V-ATPase expression in PT of normal and transgenic mice. NOD and control mice were also injected in vivo (via the tail vein) with albumin-Alexa594. After 10 minutes, the mice were killed, and their kidneys were removed and snap-frozen in liquid nitrogen, followed by 5 μm cryostat sectioning. Fluorescent albumin-positive PT were identified and captured by LCM (using a “MMI-Cell Cut” platform) (FIG. 17A).

After isolating kidney PTs and performing quantitative RT-PCR analysis, an increase in expression of both V-ATPase (FIG. 17B) and cytohesin-2 (FIG. 17C) was noted in the early stages (2-3 weeks) of type 1 diabetic NOD mice. These results were confirmed by immunocytochemistry, which revealed an increase in expression and apical redistribution of both V-ATPase (FIGS. 18A, 18B, and 18E) and megalin-receptor (FIGS. 18C, 18D, and 18E) in NOD mice with early (2-3 weeks) diabetes. Thus, these data indicate, that the EGFR/ErbB/cytohesin-2N-ATPase/aldolase pathway (FIG. 1) could be over-activated in PT during early stages of diabetes. This in turn, may lead to increased cell proliferation and over-activation of the endosomal/lysosomal pathway (FIG. 1).

Thus, as described in this disclosure, cell-permeable anti-cytohesin-2 peptides inhibit over-activation of this pathway and PT-cell proliferation during early stages of type 1 diabetes, and therefore are useful for treating or reducing a risk of developing DN.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating cancer, the method comprising:
selecting a subject having or at risk for developing cancer; and
administering to the subject a therapeutically effective amount of a cytohesin-2 inhibitor, wherein the cytohesin-2 inhibitor comprises:
a peptide consisting of an amino acid sequence that has at least 90% identity to the amino acid sequence of SEQ ID NO:9, 11, 13, 15, 25, 27, 29, or 31;
a fusion polypeptide comprising a first portion consisting of an amino acid sequence that has at least 90% identity to the amino acid sequence of SEQ ID NO:9, 11, 13, 15, 25, 27, 29, or 31; and at least a second portion linked to the first portion, wherein the second portion comprises one or more non-VATPase sequences;
or any combination thereof,
to thereby treat cancer in the subject.

2. The method of claim 1, wherein the second portion of the fusion polypeptide comprises a cell-penetrating peptide or a small molecule.

3. The method of claim 2, wherein the cell-penetrating peptide is a TAT peptide.

4. The method of claim 2, wherein the small molecule is selected from the group consisting of SecinH3, Secin16, Secin69, Secin107, and Secin132.

5. The method of claim 1, wherein the cancer is an epithelial cancer or carcinoma.

6. The method of claim 5, wherein the epithelial cancer or carcinoma is selected from the group consisting of lung cancer, pancreatic cancer, squamous cell carcinomas of the head and neck, prostate cancer, breast cancer, colon cancer, kidney cancer, liver cancer, and brain cancer.

7. The method of claim 1, the method further comprising treating the subject with chemotherapy.

8. The method of claim 1, wherein the subject is a human.

9. A method of treating or reducing a risk of developing diabetic nephropathy, the method comprising:
selecting a subject having or at risk for developing diabetic nephropathy; and
administering to the subject a therapeutically effective amount of a cytohesin-2 inhibitor, wherein the cytohesin-2 inhibitor comprises:
a peptide consisting of an amino acid sequence that has at least 90% identity to the amino acid sequence of SEQ ID NO:9, 11, 13, 15, 25, 27, 29, or 31;
a fusion polypeptide comprising a first portion consisting of an amino acid sequence that has at least 90% identity to the amino acid sequence of SEQ ID NO:9, 11, 13, 15, 25, 27, 29, or 31; and at least a second portion linked to the first portion, wherein the second portion comprises one or more non-VATPase sequences;
or any combination thereof,
to thereby treat or reduce a risk of developing diabetic nephropathy in the subject.

10. The method of claim 9, wherein the second portion of the fusion polypeptide comprises a cell-penetrating peptide or a small molecule.

11. The method of claim 10, wherein the cell-penetrating peptide is a TAT peptide.

12. The method of claim 10, wherein the small molecule is selected from the group consisting of SecinH3, Secin16, Secin69, Secin107, and Secin132.

13. The method of claim 9, wherein the subject has type 1 diabetes.

14. The method of claim 9, the method further comprising administering insulin to the subject.

15. The method of claim 8, wherein the subject is a human.

16. A method of delivering a compound into a cell, the method comprising:
providing a cell; and
contacting the cell with:
a fusion polypeptide comprising a first portion consisting of an amino acid sequence that has at least 90% identity to the amino acid sequence of SEQ ID NO: 11 or 27; and at least a second portion linked to the first portion, wherein the second portion comprises a TAT peptide; and
a compound selected from the group consisting of a nucleic acid, amino acid, peptide, polypeptide, antibody, small molecule, toxin, and nanoparticle.

17. The method of claim 16, wherein the cell is contacted with:
a fusion polypeptide comprising a first portion consisting of an amino acid sequence that has at least 90% identity to the amino acid sequence of SEQ ID NO: 11; and at least a second portion linked to the first portion, wherein the second portion comprises a TAT peptide; and
a fusion polypeptide comprising a first portion consisting of an amino acid sequence that has at least 90% identity to the amino acid sequence of SEQ ID NO:27; and at least a second portion linked to the first portion, wherein the second portion comprises a TAT peptide.

18. The method of claim 16, wherein the compound is linked to the fusion polypeptide.
